SEQUENCE LISTING (10 sequences)

SEQ ID NO: 1 - RNA, 197 nt, artificial
  ARTIFICAL SEQUENCE VARIANT FROM A VIRUS
gggguuuccaaccggaauuugagggaugccuaggcaucccccgugcgucccuuuacgagggauugucgacucuagucgacgucugggcgaaaaauguacgagaggaccuuuucgguacagacgguaccugaggguaugccuaggcaucccccgcgccgguuucggaccuccagugcguguuaccgcacugucgaccc

SEQ ID NO: 2 - DNA, 221 bp, artificial
  ARTIFICAL SEQUENCE VARIANT FROM A VIRUS
ggggaccccccggaaggggggacgaggtgcgggcacctcgtacgggagttcgaccgtgacgagtcacgggctagcgctttcgcgctctcccaggtgacgcctcgtgaagaggcgcgaccttcgtgcgtttcggcgacgcacgagaaccgccacgctgcttcgcagcgtggccccttcgcgcagcccgctgcgcgaggtgacccccgaaggggggttcccca

SEQ ID NO: 3 - DNA, 86 bp, artificial
  ARTIFICAL SEQUENCE VARIANT FROM A VIRUS
gggttcatagcctattcggcttttaaaggacctttttccctcgcgtagctagctacgcgaggtgaccccccgaaggggggtgcccc

SEQ ID NO: 4 - DNA, 130 bp, artificial
  ARTIFICAL SEQUENCE VARIANT FROM A VIRUS
gggttcatagcctattcggcttcgcgcatgggaatttgagggacgatggggaagtgggagcgcgttttaaaggacctttttccctcgcgtagctagctacgcgaggtgaccccccgaaggggggtgcccc

SEQ ID NO: 5 - DNA, 118 bp, artificial
  ARTIFICAL SEQUENCE VARIANT FROM A VIRUS
gggttcatagcctattcggcttcgcgcccgtttataatacttagtgagcgcgttttaaaggacctttttccctcgcgtagctagctacgcgaggtgaccccccgaaggggggtgcccc

SEQ ID NO: 6 - DNA, 118 bp, artificial
  ARTIFICAL SEQUENCE VARIANT FROM A VIRUS
gggttcatagcctattcggcttcgcgcccctggggtttgcctcaggagcgcgttttaaaggacctttttcccttgcgtagctagctacgcgaggtgaccccccgaaggggggtgcccc

SEQ ID NO: 7 - RNA, 26 nt, Artificial Sequence
  ARTIFICAL SEQUENCE VARIANT FROM A VIRUS
gggaauuugaggacgaugguaagugg

SEQ ID NO: 8 - RNA, 29 nt, Artificial
  ARTIFICAL SEQUENCE VARIANT FROM A VIRUS
gggaauuugaggaacgauagguaagugga

SEQ ID NO: 9 - RNA, 55 nt, Bacillus subtilis
ggccccguaacuucgggagaaggggugcucuguuagggugcaagcccgagagagc

SEQ ID NO: 10 - RNA, 157 nt, artificial
  ARTIFICAL SEQUENCE VARIANT FROM A VIRUS
gggguuucgaaccggaauuugagggaugccuaggcaucccccgugcgucccuuuacgagggauugucgacucuagaggauccgguaccugagggaugccuaggcauccccgcgcgccgguuucggaccuccagugcguguuaccgcacugucgaccc

(19) United States
(12) Patent Application Publication
(10) Pub. No.: US 2006/0257904 A1
(43) Pub. Date: Nov. 16, 2006

(54) Agglomeration protein cascades, compositions and methods regarding the same

(75) Inventors: Abraham Grossman, Pleasantville, NY (US); James Moe, New York, NY (US); Sara Vasan, New York, NY (US); Eliot Davidowitz, West Hempstead, NY (US)

Correspondence Address: DAVIDSON, DAVIDSON & KAPPEL, LLC, 485 SEVENTH AVENUE, 14TH FLOOR, NEW YORK, NY, 10018, US

(73) Assignee: Q-RNA, Inc., New York, NY (US)

(21) Appl. No.: 11/400,808

(22) Filed: Apr. 7, 2006

Related U.S. Application Data
(60) Provisional application No. 60/669,239, filed on Apr. 7, 2005.
(60) Provisional application No. 60/724,568, filed on Oct. 7, 2005.
(60) Provisional application No. 60/788,862, filed on Apr. 3, 2006.

(51) Int. Cl. C12Q 1/68; C07H 21/04; C12P 21/06; C12M 1/34

(52) U.S. Cl. 435/6; 435/69.1; 435/320.1; 435/325; 530/350; 536/23.5; 435/287.2

(57) ABSTRACT

Described herein are compositions and methods for identifying cellular factors involved in protein agglomeration. One such factor is a nucleic acid component. Another factor is a cellular binding factor. The nucleic acid components, and methods of using them, to interact with agglomeration proteins are also disclosed herein. The nucleic acid compositions herein comprise one or more DNA or RNA molecules having affinity for at least one agglomeration protein. The nucleic acid component is a naturally or non-naturally occurring molecule with twenty or more ribonucleotide bases. For RNA, at least one nucleotide sequence portion of this RNA molecule has affinity to at least one consensus sequence present in the agglomeration RNA-binding protein. Methods disclosed herein are directed towards detecting the presence of one or more agglomeration proteins in a sample matrix using the amplibody compositions described herein. Also described herein is an in vitro system (“TRIPARTITE”), which utilizes a NA and a non-NA chaperone to analyze the amyloid disease progression and test drugs potentially useful in combating such diseases. Also described are in vivo, transgenic animal models for analyzing amyloid diseases and agents potentially useful in combating those diseases.

This application claims priority to U.S. Provisional Application No. 60/669,239, filed Apr. 7, 2005; U.S. Provisional Application No. 60/724,568, filed Oct. 7, 2005; and U.S. Provisional Application entitled “Methods of Detection of Neurodegenerative Diseases and Compounds for the Prevention and Treatment Thereof”, filed Apr. 2, 2006 with Express Mail Label No. EV319074198US, identified as Docket No. 567.1031P, the dislocures of which are hereby incorporated by reference in their entireties.

### FIELD OF THE INVENTION

The present invention pertains to compositions and methods used to interact with proteins, including RNA and other cellular factors or “chaperones” involved in the processes that lead to protein misfolding. The present invention also relates to the field of agglomeration proteins, the process of misfolding of proteins and diseases associated with such agglomeration and misfolded proteins. Embodiments of the present invention relate to compositions, methods, non-naturally occurring animals and model systems for determining factors capable of participating in and potentially altering agglomeration disease processes. Other embodiments of the present invention relate to methods, devices and kits for separating, determining the presence of or isolating a protein associated with agglomeration disease.

### BACKGROUND OF THE INVENTION

Millions of individuals will succumb to Alzheimer's disease and other, often fatal, neurodegenerative disorders and face a future of increasing debilitation and consequential suffering for themselves and their families. These are frequently referred to as amyloid diseases or protein misfolding diseases. Examples of some of these diseases include transmissible spongiform encephalopathies (TSE) (e.g., scrapie in sheep, bovine spongiform encephalopathy (BSE) in cow (“Mad Cow” disease) and variant Creutzfeldt-Jakob disease in humans), neurodegenerative disorders (e.g., Alzheimer's and Parkinson's disease), metabolic disorders (e.g., Type II diabetes), respiratory disorders (e.g., cystic fibrosis), and ocular maladies (e.g., cataracts).

Clues to the molecular mechanism of these diseases initially came from the study of diseases such as Mad Cow disease, which has similar characteristics to Alzheimer's. With respsect to TSEs, such as Mad Cow disease, an improperly folded form of the prion protein (PrP) is thought to be the causative agent.

In an effort to control the spread of Mad Cow disease in Europe, entire herds of cattle were destroyed if a single infected cow was found infected. Currently, the United States is facing an analogous situation with the rapid spread of chronic wasting disease, the prion disease of deer and elk. Although there is no evidence that chronic wasting disease is transmissible to humans, it is the only prion disease of free-ranging animals and is threatening the population of deer and elk, particularly in states like Wisconsin where extermination of all resident herds is being considered. Because the clinical manifestations of some of these diseases, such as Creutzfeldt-Jakob disease in humans, can take decades to develop, donated human blood, organs and tissue may be contaminated without the knowledge of the donor or recipient. These medical and economic circumstances make critical the need for the development of sensitive diagnostics for these types of diseases.

Efforts to systematically identify valid drug targets, a first step toward finding effective drugs, have failed to date. Recent discoveries in the scientific community together with work by disclosed in the present invention reveal the molecular mechanism of amyloid or protein misfolding diseases and the pathogenic pathway shared by these diseases. Thus, the present invention provides novel and integrated drug discovery systems for target identification, drug discovery, and multifaceted validation, enabling development of effective diagnostics and drugs for Alzheimer's disease and other disorders that have no effective therapies today.

### Misfolded Proteins: Etiology of Neurodegenerative and Other Amyloid Diseases

Correctly functioning proteins are formed from chains of amino acids that must be properly folded into a three-dimensional structure. For decades, scientists have investigated the mechanisms of protein folding to define the relationship of protein structure to biological function. All animals, including humans, have mechanisms within cells for making normal proteins and for destroying or breaking misshapen (misfolded) proteins into small constituent parts, which can, in turn, be recycled. Thus, little effort was spent on misfolded proteins, which were thought to merely degrade.

In one such mechanism for destroying and recycling misshapen proteins, the cell tags the misfolded proteins with a molecule called ubiquitin and carries them to a barrel-shaped structure called the proteosome, where they are then broken down into their constituents for recycling. However, aging cells may not have the energy to run the proteosome or ubiquitin system. Toxins, inflammation, gene mutations and trauma can also create imbalances, thus overwhelming the cell's system for disposing of misfolded proteins.

Only in the last decade did the scientific community understand that misfolded proteins are centrally involved in a multitude of very serious human diseases, including those mentioned above. These amyloid diseases are thought to have a common mechanism of pathogenesis—namely, the misfolding of soluble cellular proteins or fragments into insoluble and aggregated, or self-assembled, abnormalities, referred to as fibrils and plaques. Thus, when the processes, which normally degrade misfolded proteins, go awry small numbers of these misfolded proteins accumulate to form pathological agglomerations or fibrils by first forming tiny spherical particles which, over time, begin sticking together to form filaments and fibrils. These accumulate into insoluble deposits of protein called amyloid plaque. These plaques, fibril assemblies or agglomerations are manifest upon dissection of subjects afflicted with neuronal cell death and brain wasting diseases such as Alzheimer's and Parkinson's disease in humans, scrapie, mad cow disease and chronic wasting diseases in animals. Misfolded proteins also appear to damage cells in the lung, heart, kidney, pancreas and other organs.

Until recently, researchers believed that the fibrils and plaque were themselves the bad actors, and they have been seeking medications and vaccines to remove them or block their formation. But such therapies could make some diseases worse in that plaque removal might alter the kinetics of the system and hasten the rate of production of the toxic spheres. The new picture of amyloid diseases evolved as physicists and chemists began using their research tools to understand exactly how proteins fold.

In spongiform encephalopathies, prion proteins are thought to be the etiologic agent.

Prion-based diseases result from “infectious proteins” that are cellular benign prion proteins misfolded into an infectious isoform. Alzheimer's disease is perhaps the best known protein misfolding disease, affecting four million Americans. As the disease progresses, plaque clusters made of a misfolded protein called “a-beta” accumulate in areas of the brain that control memory, mood and spatial awareness. Parkinson's disease shows similar characteristics; a protein called alpha-synuclein misfolds and aggregates in the brain in plaques called Lewy bodies. Protofibrils of synuclein have been detected in patients' cells and appear to be toxic.

Now, some researchers are linking misfolded proteins to a growing list of conditions, including Huntington's disease and A.L.S. (amyotrophic lateral sclerosis or Lou Gehrig's disease). Blood can carry misfolded proteins and a plasma protein called transthyretin often misfolds because of at least 80 mutations affecting different parts of the body. These mutations can lead to plaque buildup in the hands, feet, liver or heart. Some experts believe that a transthyretin mutation common in African-Americans leads to a form of heart disease that is difficult to diagnose.

### “Chaperones” and Amyloid Diseases

Recent discoveries have led to the concept that a newly identified group of normal cellular metabolites (proteins, glycopeptides, DNAs, RNAs, lipids, etc.) play an important role in the molecular and cellular pathogenesis of misfolded protein diseases. These molecular facilitators are collectively referred to as “chaperones.” Chaperones form protective envelopes around proteins as they fold. In the crowded cell, chaperones help prevent partly folded proteins from bumping into other proteins that may try to swap elements or otherwise interfere with proper folding. Once a protein is folded and ready to go to work, the chaperone pops off and finds another unfolded protein to protect. Chaperones can also grab misfolded intermediates and restart their folding process at the beginning, or bind aggregatable proteins and either facilitate or inhibit their misfolding and aggregation. Thus, molecular chaperones foster or reverse disease progression and represent a whole new class of targets for therapeutic intervention in diseases caused by protein misfolding.

Recent evidence indicates that amyloids share common structural properties that are largely determined by their generic polymer properties and that soluble amyloid oligomers may represent the primary pathogenic structure, rather than the mature amyloid fibrils. Since protein function is determined by the three-dimensional structure, the fact that amyloids share generic structures implies that they may also share a common pathological function. Amyloid oligomers from several different proteins share the ability to permeabilize cellular membranes and lipid bilayers, indicating that this may represent the primary toxic mechanism of amyloid pathogenesis. This suggests the membrane permeabilization may initiate a core sequence of common pathological events leading to cell dysfunction and death that is shared among degenerative diseases, whereas pathological events that are unique to one particular type of amyloid or disease may lie in up stream pathways leading to protein mis-folding. Although, these upstream events may be unique to a particular disease related protein, their effects can be rationalized as having a primary effect of increasing the amount of mis-folded, potentially amyloidogenic proteins.

Amyloid oligomers also display a common structural motif that is distinct from fibrils based on the observation that a confirmation dependent antibody specifically recognizes a common epitope on amyloid oilgomers, but not fibrils, monomers or natively folded proteins for many different types of proteins. This indicates that the antibody recognizes a generic polypeptide backbone epitope that is independent of the amino acid sequence, but yet is shared in common among all types of amyloid oligomers. This anti-oligomer antibody also generically inhibits the toxicity of soluble oligomers examined in vitro.

Since different amyloid oligomers share a common structure and they are generically toxic to cells, this predicts that hey have the same primary mechanism in toxicity in generative diseases.

A major impediment to drug discovery for protein misfolding or amyloid diseases is the failure to identify and validate appropriate targets. In retrospect, this is not surprising, as the industry's conventional disease-focused approach (1) does not recognize that different diseases with diverse etiologies have a common pathogenic mechanism and (2) depends upon finding an “active protein” in the disease pathway. It is only recently that the scientific community understood that the amyloid cascade is composed of components that are not “active proteins,” but nonetheless interact to form aggregates and, ultimately, a variety of diseases.

The current invention meets the need for compositions and methods that can identify the molecular facilitators, or chaperones, that affect the disease cascade, as well as methods and kits that can separate proteins that participate in forming agglomeration products. Identification of these components of amyloid diseases permits a definitive identification of pathological conditions and, therefore can also help identify agents which interfere with the formation of the misfolded proteins and/or agglomeration complex, or alter the stability of such a complex.

### OBJECTS AND SUMMARY OF THE INVENTION

### Brief Description of the Figures

FIG. 1(a) depicts one embodiment of secondary structure for MDV RNA, and (b) depicts a second embodiment of secondary structure for MDV RNA;

FIG. 2 depicts a secondary structure for MNV RNA;

FIG. 3 depicts a secondary structure for MNV:AP1 RNA;

FIG. 4 depicts a secondary structure for MNV-1 RNA;

FIG. 5 depicts a secondary structure for MNV-2 RNA,

FIG. 6 depicts a secondary structure for RQ11+12 RNA;

FIG. 7 shows a gel depicting the amplification of MNV and PrP-Amp;

FIG. 8(a) shows a gel demonstrating the specificity of MNV and PrP-Amp binding to a prion protein, (b) represents the same binding data in graphical form;

FIG. 9 is cartoon depiction of prion protein's two binding sites;

FIG. 10 is a graph representing competition binding data between PrP-Amp and either AP1 or MNV;

FIG. 11 is a graph showing competition binding data between RQ11+12 and AP1;

FIG. 12 shows the secondary structure for (a) AP1, (b) nucleotides 22-53 of MNVLO, (c) MNVUP, (d) BS1577, (e) MNV, (f) RQT157, and (g) RQ11+12;

FIG. 13 is an RNA-RNA gel shift;

FIG. 14 is a graph showing the results from a competition study of MNV:AP1 and RQ11+12 binding to hrPrP by AP1 and MNV;

FIG. 15(a) shows the topology of hrPrP, and (b) illustrates graphical data for the binding of RNA to truncated hrPrP;

FIG. 16 shows results from an RnaseA protection study;

FIG. 17 illustrates data demonstrating that RQ11+12 binds specifically to PrP where (a) shows the results of a filter binding assay, and (b) shows the results of a gel shift assay;

FIG. 18(a) shows an electron micrograph with hrPrP, and (b) hrPrP and RQ11+12 RNA;

FIG. 19 is an electron micrograph of 4 uM of Tau412 in 1× fibril buffer after 3 hours of incubation;

FIG. 20 is an electron micrograph of 4 uM of Tau412 in 1× fibril buffer after 3 hours of incubation;

FIG. 21 (A-C) depicts a western blot analysis of a column cartridge which demonstrates the ability of RQ11+12 to bind more than a single PrP peptide;

FIG. 22 (A-E) depicts a western blot analysis of a column cartridge containing adsorbents impregnated with RNA;

FIG. 23 depicts the RNAs SC-2 (A), and a variant containing a point mutation, SC-4(B);

FIG. 24 depicts a gel shift assay showing that only RQ11+12 efficiently converts hrPrP to a Proteinase K resistant form in the presence of BCS;

FIG. 25 depicts a gel shift assay showing the monoclonal antibody (mAb) 7D9, directed against recombinant mouse PrP(A) and an extract that interacts with RQ11+12 is PrP (B);

FIG. 26 depicts a gel assay using brain homogenates from mouse, rat and hamster to determine if RQ11+12 can interact with endogenous PrPC from organisms other than mouse;

FIG. 27 depicts a gel assay showing how multiple PrP proteins bind to a single RQ11+12 RNA;

FIG. 28 depicts a gel assay showing that only RQ11+12 efficiently converts hrPrP to a Proteinase K resistant form in the presence of BCS;

FIG. 29 depicts a gel assay showing the removal of BCS from the incubation mixture resulted in complete digestion of hrPrP;

FIG. 30. depicts a gel assay which shows that Q-factor presumably induces further structural changes to the RNA/PrP complex that are already extensive as demonstrated by the lack of RNA degradation by RNase A when in complex with hrPrP;

FIG. 31 depicts a gel assay of nucleoprotein complexes subjected to digestion by ribonuclease A (RNase A);

FIG. 32 depicts the separation of RNA-induced PrP-Oligomers by gel filtration;

FIG. 33 depicts the recovery of PrP in G-100 peaks from two experiments;

FIG. 34 depicts the elution pattern of PrPRQ and PrP control from Sephadex G-100;

FIG. 35 depicts a normal sheep brain extract incubated with RQT157 or RQ11+12 (ELISA);

FIG. 36 depicts Nsh br ex incubated with various types of RNAs (ELISA);

FIG. 37 depicts a PrP-oligomer assay in normal and scrapie sheep brain (ELISA);

FIG. 38 depicts PrP Oligomers in normal and scrapie brain extracts (ELISA);

FIG. 39 depicts PrP+RQ (ELISA);

FIG. 40 depicts a graph of rPrP incubated with or without RQ11+12;

FIG. 41 depicts a graph showing detection of nanogram quantities of human Tau target protein in fewer than 20 minutes at 37° C. using the Immuno Q-Amp method and testing a blocking reagent;

FIG. 42 depicts a graph showing detection of nanogram quantities of human Tau target protein in fewer than 20 minutes at 37° C. using the Immuno Q-Amp method and testing a different blocking reagent. These results show high sensitivity and very low background signal;

FIG. 43 depicts SEQ ID NO. 7;

FIG. 44 depicts typical results of prion protein fragments following protease K treatment;

FIG. 45 depicts typical results of prion protein fragments following protease K treatment;

FIG. 46 depicts the efficacy of chlorpromazine in blocking the PrP protection by serum or lipoproteins in a dose-dependent manner;

FIG. 47 depicts the accumulation of amyloid deposits in the brains of Drosophila;

FIG. 48 depicts data obtained using Tau protein which demonstrate that the chaperones of the PrP model system misfold Tau protein into an isoform with amyloid characteristics;

FIG. 49 depicts data obtained using Tau protein which demonstrate that the chaperones of the PrP model system misfold Tau protein into an isoform with amyloid characteristics;

FIG. 50 (A-I) depicts an assay where Q-Factor from bovine calf serum (BCS) or human plasma (HP) facilitating transformation was tracked during steps of purification;

FIG. 51 (A) depicts a resolution by SDS PAGE for MALDITOF spectrometry; (B) is a graph showing dose dependent facilitation of PrPsen to PrPRes transformation, thus, identifying Q-factor as α2M;

FIG. 52 (A-D) depicts gel assays of the effect of α2M on PK activity;

FIG. 53 (A-P) depicts the structure of complexes formed by rhuPrP-Cy5, RQ11+12 RNA and α2M;

FIG. 54 depicts spectrophotometry of different Tau-RNA complexes;

FIG. 55 depitcs the formation of fibrils of tau upon addition of RQT157(A); and the aggregation of tau upon addition of RQ11+12(B);

FIG. 56 depicts light microscopy of Tau/RNA complexes;

FIG. 57 depicts gel assay results of example 15;

FIG. 58 depicts gel assay results of example 15;

FIG. 59 depicts gel assay results of example 16;

FIG. 60 depicts gel assay results of example 16;

FIG. 61 depicts gel assay results of example 16;

FIG. 62 depicts gel assay results of example 17;

FIG. 63 depicts gel assay results of example 18;

FIG. 64 depicts gel assay results bf example 18;

FIG. 65 depicts gel assay results of example 19;

FIG. 66 depicts gel assay results of example 19;

FIG. 67 depicts gel assay results of example 20;

FIG. 68 depicts gel assay results of example 20;

FIG. 69 depicts gel assay results of example 21;

FIG. 70 depicts gel assay results of example 21;

FIG. 71 depicts gel assay results of example 22;

FIG. 72 depicts a flow chart for example 2.

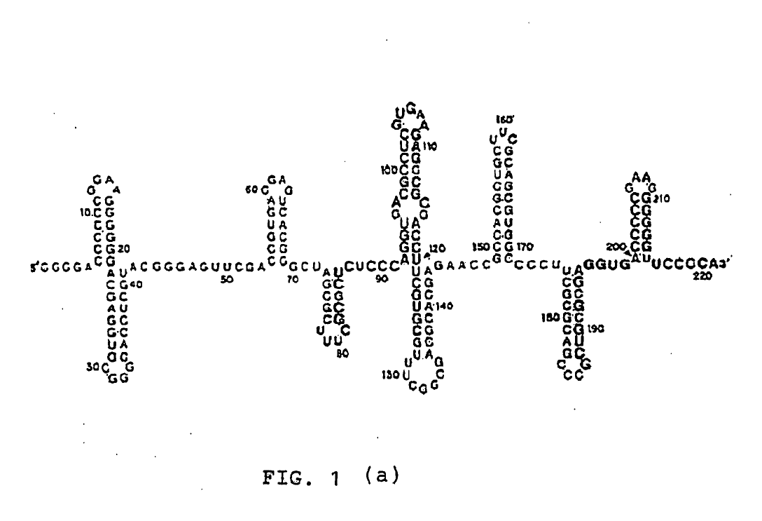

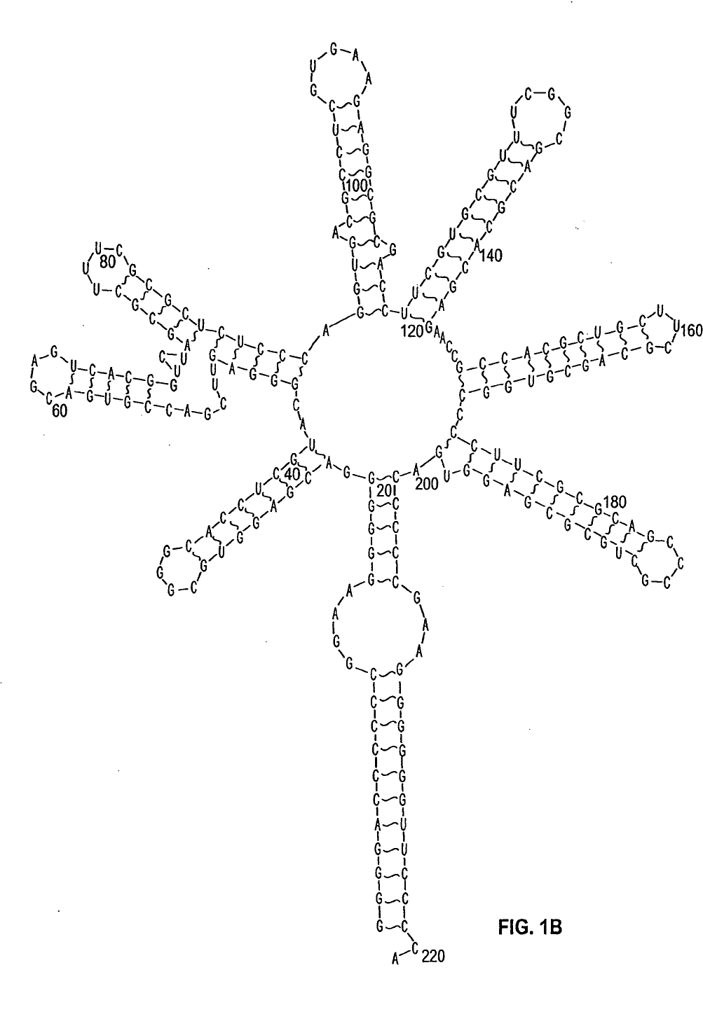

### DETAILED DESCRIPTION

To facilitate the understanding of the present invention, a number of terms and phrases are defined below:

As used herein, the term “nucleic acid molecule” is intended to include DNA molecules, e.g., cDNA or genomic DNA, and RNA molecules, e.g., mRNA, and analogs of the DNA or RNA generated using nucleotide analogs. The nucleic acid molecule can be single-stranded or double-stranded, but preferably is double-stranded DNA.

The term “isolated nucleic acid molecule” includes nucleic acid molecules, which are separated from other nucleic acid molecules that are present in the natural source of the nucleic acid. For example, with regards to genomic DNA, the term “isolated” includes nucleic acid molecules that are separated from the chromosome with which the genomic DNA is naturally associated. Preferably, an “isolated” nucleic acid is free of sequences which naturally flank the nucleic acid (i.e., sequences located at the 5′ and 3′ ends of the nucleic acid) in the genomic DNA of the organism from which the nucleic acid is derived. For example, in various embodiments, the isolated marker nucleic acid molecule of the invention, or nucleic acid molecule encoding a polypeptide marker of the invention, can contain less than about 5 kb, 4 kb, 3 kb, 2 kb, 1 kb, 0.5 kb or 0.1 kb of nucleotide sequences which naturally flank the nucleic acid molecule in genomic DNA of the cell from which the nucleic acid is derived. Moreover, an “isolated” nucleic acid molecule, such as a cDNA molecule, can be substantially free of other cellular material, or culture medium when produced by recombinant techniques, or substantially free of chemical precursors or other chemicals when chemically synthesized.

As used herein, a “naturally-occurring” nucleic acid molecule includes an RNA or DNA molecule having a nucleotide sequence that occurs in nature, e.g., encodes a natural protein.

As used herein, the terms “polynucleotide” and “oligonucleotide” are used interchangeably, and include polymeric forms of nucleotides of any length, either deoxyribonucleotides or ribonucleotides, or analogs thereof. Polynucleotides can have any three-dimensional structure, and can perform any function, known or unknown. The following are non-limiting examples of polynucleotides: a gene or gene fragment, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide can comprise modified nucleotides, such as methylated nucleotides and nucleotide analogs. If present, modifications to the nucleotide structure can be imparted before or after assembly of the polymer. The sequence of nucleotides may be interrupted by non-nucleotide components. A polynucleotide may be further modified after polymerization, such as by conjugation with a labeling component. The term also includes both double- and single-stranded molecules. Unless otherwise specified or required, any embodiment of this invention that is a polynucleotide encompasses both the double-stranded form and each of two complementary single-stranded forms known or predicted to make up the double-stranded form.

A polynucleotide is composed of a specific sequence of four nucleotide bases: adenine (A); cytosine (C); guanine (G); thymine (T); and uracil (U) for thymine when the polynucleotide is RNA. Thus, the term “polynucleotide sequence” is the alphabetical representation of a polynucleotide molecule. This alphabetical representation can be inputted into databases in a computer having a central processing unit and used for bioinformatics applications such as functional genomics and homology searching.

A “gene” includes a polynucleotide containing at least one open reading frame that is capable of encoding a particular polypeptide or protein after being transcribed and translated. Any of the polynucleotide sequences described herein may be used to identify larger fragments or full-length coding sequences of the gene with which they are associated. Methods of isolating larger fragment sequences are known to those of skill in the art, some of which are described herein.

A “gene product” includes an amino acid, e.g., peptide or polypeptide, generated when a gene is transcribed and then translated.

A “primer” includes a short polynucleotide, generally with a free 3′-OH group that binds to a target or “template” present in a sample of interest by hybridizing with the target, and thereafter promoting polymerization of a polynucleotide complementary to the target. A “polymerase chain reaction” (“PCR”) is a reaction in which replicate copies are made of a target polynucleotide using a “pair of primers” or “set of primers” consisting of “upstream” and a “downstream” primer, and a catalyst of polymerization, such as a DNA polymerase, typically a thermally-stable polymerase enzyme. Methods for PCR are well known in the art, and are taught, for example, in MacPherson et al., IRL Press at Oxford University Press (1991). All processes of producing replicate copies of a polynucleotide, such as PCR or gene cloning, are collectively referred to herein as “replication”. A primer can also be used as a probe in hybridization reactions, such as Southern or Northern blot analyses (see, for example, Sambrook, J., Fritsh, E. F., and Maniatis, T. Molecular Cloning: A Laboratory Manual 2nd, ed., Cold Spring Harbor Laboratory, Cold Spring Harbor Laboratory Press, Cold Spring Harbor, N.Y., 1989).

The term “cDNAs” includes complementary DNA, that is mRNA molecules present in a cell or organism made into cDNA with an enzyme such as reverse transcriptase. A “cDNA library” includes a collection of mRNA molecules present in a cell or organism, converted into cDNA molecules with the enzyme reverse transcriptase, then inserted into “vectors” (other DNA molecules that can continue to replicate after addition of foreign DNA). Exemplary vectors for libraries include bacteriophage, viruses that infect bacteria, e.g., λ phage. The library can then be probed for the specific cDNA (and thus mRNA) of interest.

A “delivery vehicle” includes a molecule that is capable of inserting one or more polynucleotides into a host cell. Examples of delivery vehicles are liposomes, biocompatible polymers, including natural polymers and synthetic polymers; lipoproteins; polypeptides; polysaccharides; lipopolysaccharides; artificial viral envelopes; metal particles; and bacteria, viruses and viral vectors, such as baculovirus, adenovirus, and retrovirus, bacteriophage, cosmid, plasmid, fungal vector and other recombination vehicles typically used in the art which have been described for replication and/or expression in a variety of eukaryotic and prokaryotic hosts. The delivery vehicles may be used for replication of the inserted polynucleotide, gene therapy as well as for simply polypeptide and protein expression.

A “vector” includes a self-replicating nucleic acid molecule that transfers an inserted polynucleotide into and/or between host cells. The term is intended to include vectors that function primarily for insertion of a nucleic acid molecule into a cell, replication vectors that function primarily for the replication of nucleic acid and expression vectors that function for transcription and/or translation of the DNA or RNA. Also intended are vectors that provide more than one of the above function.

A “host cell” is intended to include any individual cell or cell culture that can be or has been a recipient for vectors or for the incorporation of exogenous nucleic acid molecules, polynucleotides and/or proteins. It also is intended to include progeny of a single cell. The progeny may not necessarily be completely identical (in morphology or in genomic or total DNA complement) to the original parent cell due to natural, accidental, or deliberate mutation The cells may be prokaryotic, include but are not limited to bacterial cells.

The term “genetically modified” includes a cell containing and/or expressing a foreign gene or nucleic acid sequence that in turn modifies the genotype or phenotype of the cell or its progeny. This term includes any addition, deletion, or disruption to a cell's endogenous nucleotides.

As used herein, “expression” includes the process by which polynucleotides are transcribed into mRNA and translated into peptides, polypeptides, or proteins. If the polynucleotide is derived from genomic DNA, expression may include splicing of the mRNA, if an appropriate eukaryotic host is selected. Regulatory elements required for expression include promoter sequences to bind RNA polymerase and transcription initiation sequences for ribosome binding. For example, a bacterial expression vector includes a promoter such as the lac promoter and for transcription initiation the Shine-Dalgarno sequence and the start codon AUG (Sambrook, J., Fritsh, E. F., and Maniatis, T. Molecular Cloning: A Laboratory Manual. 2nd, ed, Cold Spring Harbor Laboratory, Cold Spring Harbor Laboratory Press, Cold Spring Harbor, N.Y. 1989). Similarly, a eukaryotic expression vector includes a heterologous or homologous promoter for RNA polymerase II, a downstream polyadenylation signal, the start codon AUG, and a termination codon for detachment of the ribosome. Such vectors can be obtained commercially or assembled by the sequences described in methods well known in the art, for example, the methods described below for constructing vectors in general.

“Differentially expressed”, as applied to a gene, includes the differential production of mRNA transcribed from a gene or a protein product encoded by the gene. A differentially expressed gene may be overexpressed or underexpressed as compared to the expression level of a normal or control cell. In one aspect, it includes a differential that is 2.5 times, preferably 5 times or preferably 10 times higher or lower than the expression level detected in a control sample. The term “differentially expressed” also includes nucleotide sequences in a cell or tissue which are expressed where silent in a control cell or not expressed where expressed in a control cell. A “non-coding RNA” is an RNA that does not carry information about protein composition and structure.

The term “polypeptide” includes a compound of two or more subunit amino acids, amino acid analogs, or peptidomimetics. The subunits may be linked by peptide bonds. In another embodiment, the subunit may be linked by other bonds, e.g., ester, ether, etc. As used herein the term “amino acid” includes either natural and/or unnatural or synthetic amino acids, including glycine and both the D or L optical isomers, and amino acid analogs and peptidomimetics. A peptide of three or more amino acids is commonly referred to as an oligopeptide. Peptide chains of greater than three or more amino acids are referred to as a polypeptide or a protein.

“Hybridization” includes a reaction in which one or more polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these. A hybridization reaction may constitute a step in a more extensive process, such as the initiation of a PCR reaction, or the enzymatic cleavage of a polynucleotide by a ribozyme.

Hybridization reactions can be performed under conditions of different “stringency.” The stringency of a hybridization reaction includes the difficulty with which any two nucleic acid molecules will hybridize to one another. Under stringent conditions, nucleic acid molecules at least 60%, 65%, 70%, 75% identical to each other remain hybridized to each other, whereas molecules with low percent identity cannot remain hybridized. A preferred, non-limiting example of highly stringent hybridization conditions are hybridization in 6× sodium chloride/sodium citrate (SSC) at about 45° C., followed by one or more washes in 0.2×SSC, 0.1% SDS at 50° C., preferably at 55° C., more preferably at 60° C., and even more preferably at 65° C.

As used herein, the term “hybridizes under stringent conditions” is intended to describe conditions for hybridization and washing under which nucleotide sequences at least 60% homologous to each other typically remain hybridized to each other. Preferably, the conditions are such that sequences at least about 70%, more preferably at least about 80%, even more preferably at least about 85% or 90% homologous to each other typically remain hybridized to each other. Such stringent conditions are known to those skilled in the art and can be found in Current Protocols in Molecular Biology, John Wiley & Sons, N.Y. (1989), 6.3.1-6.3.6. A preferred, non-limiting example of stringent hybridization conditions are hybridization in 6× sodium chloride/sodium citrate (SSC) at about 45° C., followed by one or more washes in 0.2×SSC, 0.1% SDS at 50° C., preferably at 55° C., more preferably at 60° C., and even more preferably at 65° C. Preferably, an isolated nucleic acid molecule of the invention that hybridizes under stringent conditions to the sequence of SEQ ID NO. 1-10.

When hybridization occurs in an antiparallel configuration between two single-stranded polynucleotides, the reaction is called “annealing” and those polynucleotides are described as “complementary”. A double-stranded polynucleotide can be “complementary” or “homologous” to another polynucleotide, if hybridization can occur between one of the strands of the first polynucleotide and the second. “Complementarity” or “homology” (the degree that one polynucleotide is complementary with another) is quantifiable in terms of the proportion of bases in opposing strands that are expected to hydrogen bond with each other, according to generally accepted base-pairing rules.

As used herein, the term “agglomeration” refers to a stable association of misfolded proteins. Meanwhile, “agglomeration proteins” as used herein, refers to proteins that can form insoluble aggregates and are the predominant protein of an agglomeration.

The terms “agglomeration” and “aggregate” are interchangeably used.

PrP, tau and other proteins are “agglomeration proteins”

the diseases can be called “protein agglomeration diseases”

the nucleo-protein complex is an “aggregate”

“Amyloid Disease” shall mean a disorder that results from accumulation of misfolded proteins, which ultimately leads to the deposition of insoluble aggregates or plaques. Accordingly, an “amyloid protein,” shall mean a protein that forms insoluble (amyloid or starch-like) aggregates in cells, tissues or organs.

“Immuno-Q-Amp”is an enzyme that can amplify certain nucleic acid molecules. Immuno-Q-Amp (I-Q-Amp) is capable of increasing the performance of standard ELISA diagnostic tests by several orders of magnitude and, thus, competes with Immuno-PCR. The inventors developed the Q-Amp enzyme, a powerful amplifier of nucleic acids providing 10¹³-fold amplification within 30 minutes at room temperature. I-Q-Amp offers the added benefit of significantly reducing the number of washing steps associated with Immuno-PCR, reducing both time to complete the assay and costs to run it. This enzyme can be used to improve the sensitivity of any number of existing or new immunological (ELISA-based) diagnostic systems.

The terms “virus” and “phage” will be used interchangeably throughout this disclosure to mean “virus and phage”. In one aspect of the invention, the RNA virus is a retrovirus. The RNA virus can be selected from the group consisting of a human immunodeficiency virus (HIV), polio virus, influenza virus, smallpox virus, chicken pox virus, Herpes virus, varicella zoster virus, Epstein-Ban virus, cytomegalovirus, feline leukemia virus (FeLV), human T cell leukemia virus (HTLV), simian immunodeficiency virus (SIV), and combinations thereof.

The invention, in its various embodiments, is the only one of its kind that is both a “biomimetic” (mimics the in vivo processes) of the protein misfolding process and systematic and thus, truly capable of being adapted to a high throughput screening system (“HTS”). While there are a number of in vitro and animal model systems that resemble some aspects of amyloid formation, none-to-date fully mimic the protein misfolding cascade found in human disease etiology.

In certain embodiments of the invention a subset of amyloid diseases is exploited—the prion diseases. The primary diagnostic marker for prion diseases is the PrP^{Sc} protein, an abnormally folded isoform of the normal cellular prion protein PrP^{C}, a 35 kDa glycoprotein. Fibrillation accompanies the change in protein structure from the α-helix-rich conformation of PrP^{C} into the β-sheet-rich conformation of PrP^{Sc}, the major protein component scrapie-associated fibrils (40,44-47). The N-terminal region of both isoforms (amino acids 23-90) lacks a preferred stable structure, as seen by NMR (48). Several biochemical traits distinguish PrP^{Sc} from PrP^{C}, such as the insolubility of PrP^{Sc} in physiological solutions, its tendency to aggregate and the resistance of its C-terminal domain (amino acids 90-231) to digestion by proteinase K (45,46). The lack of a single PrP^{Sc} specific antibody necessitates the use of cross-reactive antibodies that bind both isoforms, thus requiring that samples first be treated with proteinase K to destroy the protease-sensitive PrP^{C} isoform before testing. Nucleic acids have been shown to interact with PrP and to form stable nucleoprotein complexes both in vitro (49-53) and in vivo (40,44,54-56). In the case of some RNAs, the binding interaction occurs with a significant degree of specificity (50,53). This specificity can be applied to affinity-purify prion proteins from solutions for their concentration, removal or detection.

Using a set of artificial RNAs, differential RNA-binding activities of the human recombinant PrPC protein (hrPrP) were characterized by filter-binding assay. hrPrP has a nonspecific RNA-binding activity and forms complexes with all of the RNAs tested with dissociation constants measuring in the low nanomolar range (‘non-specific binding’). However, certain RNAs showed unique binding characteristics by maintaining high-affinity binding in the presence a vast molar excess of competing tRNA (‘specific binding’). Although the specific binding RNAs were relatively unaffected by non-specific RNA molecules such as tRNA or other RNAs in the group, they competed with each other for this discriminatory site. These results suggest a complex interaction of binding domains within PrP. The utilization of PrP peptides demonstrated that all of these events occurred along the N-terminal region of the protein. The RNA construct RQ11+12 bound tightly and specifically to hrPrP and was used to make a RNA-based filtration cartridge that demonstrated the ability to bind both PrP^{C} and PrP^{Sc} isoforms from biological solutions. The cartridge also functioned as a concentrator, allowing us to increase the level of sensitivity of standard Western analysis by three orders of magnitude.

### Facilitators of Misfolding of Proteins and Agglomeration Formation

The present invention identifies agents, or facilitators, involved in the cascade of events that involves the misfolding of proteins, which in turn, eventually lead to the formation of an insoluble aggregate.

In certain embodiments of the invention, these facilitators are nucleic acids. Essentially there are two types of nucleic acid found in living cells. One is deoxyribonucleic acid (“DNA”), and the other is ribonucleic acid (“RNA”). Under normal physiological conditions, both of these nucleic acid molecules are associated with proteins and form nucleoprotein complexes. These proteins can include scaffolding and chaperone proteins, enzymes, ligases, telomerases, etc. These nucleic acid binding proteins perform functions necessary for normal metabolism and cell/tissue viability.

In other embodiments of the invention, “binding factors” which can be other proteins, also affect the cascade. However, these binding factors may be complexes of several components. Most commonly, such binding factors are lipid and protein complexes. Although the binding factors and RNA may be found in small concentrations in the agglomeration, these constituents have a scaffolding and/or chaperone function.

In the absence of certain misfolding facilitators, nucleic acids and binding factors, the protein assumes a normal isoform. However, in the presence of certain nucleic acids and binding factors, the protein assumes a misfolded isoform. These misfolded proteins, under certain conditions and with the presence of molecular facilitators accumulate, in vivo or in vitro, and form clusters or agglomerations.

### Nucleic Acid Ligands

The invention described herein pertains in part to functional ligands and their ability to interact with target molecules. A functional ligand, as used herein, is a molecule that when bound to or interacting with a target molecule, chemically and/or biologically modifies the target molecule. For example, the interaction of a functional ligand with a target molecule can confer protein digestion resistance upon the bound target molecule in contrast to its free form that is otherwise susceptible to protease digestion. Other chemical and biological properties can be modified as well by such interaction. Many of these modifications can be detected by methods well known to those skilled in the art employing techniques extant in the art. Examples of such functional ligands include, but are not limited to, nucleic acids, peptides, proteins, lipoproteins, glycoproteins, and combinations thereof.

Nucleic acid (“NA”) probes are typically designed to identify, bind and inactivate NA target molecules. A major limitation of NA probes is that require special modification to detect protein and other non-NA cellular targets. This limitation of NAs is very important, because most proteins are thousands of times more abundant than DNA or RNA products. Very often protein targets are easier to access than NA molecules, for example, protein detection does not require laborious sample preparation. The relative high stability of proteins during sample preparation and their potential higher physiological relevance compared with DNA or RNA made them targets of choice in the development of detection assays.

A significant portion of RNA-binding proteins (“RNP”) mediate post-transcriptional regulation of gene expression. Heterogenous nuclear RNAs (“hnRNA”) are the primary transcripts of protein encoding genes. These transcripts (hnRNA) are processed in the nuclei of eukaryotic cells and, at least a portion of such hnRNAs, become messenger RNAs (“mRNAs”). From the time hnRNAs emerge from the transcriptional complex, and throughout the time they are in the nucleus, they are associated with proteins termed hnRNA proteins. Members of this family of proteins are required for multiple steps during mRNA metabolism, including pre-mRNA processing and mRNA localization, translation and stability. The majority of proteins associated with RNAs appear to be associated with hnRNAs and mRNAs in hnRNP and mRNA complexes.

The current invention employs NAs as functional ligands. Specifically, these NAs can be characterized as antibody-like (hereinafter “NA antibody”) for they specifically interact with target molecules, usually proteins.

Embodiments of the present invention comprise a multifaceted platform technology for the molecular engineering of NA ligands that demonstrate high affinity and specificity for medicinally and commercially valuable molecular targets. The basis of this technology rests on recent advances in combinatorial chemistry and in vitro evolution process that is coupled with the rational design of RNA structures and novel screening methods for their functionality. The combinatorial chemistry and in vitro evolution of RNA ligands utilize the high complexity of RNA libraries diversified with natural NA sequences. These libraries are amplifiable using Immuno-Q-Amp™ enzyme (available from Q-RNA, New York, N.Y.), a form of RNA-dependant RNA polymerase.

The present invention discloses a small, highly structured (shs)RNA that binds human recombinant prion protein (hrPrP) with high affinity and specificity under physiological conditions (e.g. 10% bovine calf serum (BCS), neutral pH, nanomolar concentrations of RNA and hrPrP). The invention also demonstrates the ability of this shsRNA to form highly stable nucleoprotein complexes with hrPrP and cellular PrP (PrPC) from various cell extracts and mammalian brain homogenates. The apparent mass of the nucleoprotein complex is dependent on the molar ratio of hrPrP to RNA during complex formation. The hrPrP in these complexes acquires resistance to degradation by Proteinase K (PK). Other shsRNAs, however, under identical conditions, neither form stable complexes with hrPrP nor do they induce resistance to PK digestion. Also demonstrated is the result that the RNAs in these nucleoprotein complexes become resistant to ribonuclease A hydrolysis. These interactions between shsRNAs and hrPrP suggest possible roles of RNAs in the modulation of PrP structure and perhaps disease development. ShsRNAs that bind to hrPrP with high affinity and induce resistance to PK digestion can be used to develop molecular biology assays for the screening of compounds associated with PrP structure transformation or for drugs that inhibit this process.

### The TRIPARTITE System

Another embodiment of this invention comprises the application of an RNA specific for amyloid proteins and another chaperone (a cellular binding factor), in an in vitro system (“the TRIPARTITE system”) capable of mimicking the transformation of soluble amyloid protein into the insoluble aggregates and plaques. In one embodiment, the TRIPARTITE system models BSE and the transformation of benign prion protein (PrP) from a proteinase K (pK) sensitive isoform to its alternative, pK resistant, isoform and serves as a powerful tool for elucidating the underlying pathogenic mechanisms of prion-related diseases (as well as other disorders caused by misfolded proteins) and discovering therapeutic modalities. This system can serve as a powerful means to the development of drug discovery tools. Experimental data provided in the Examples hereinbelow, indicate that the TRIPARTITE system models essential events of the in vivo refolding or misfolding cascade that occur during PrP conversion from a normal conformation to a scrapie disease isoform. Thus, in this embodiment, the system can serve as a powerful tool for elucidating the underlying pathogenic mechanisms of prion related diseases and also for validating or screening for identification of anti-prion compounds.

With respect to TSEs, several hypotheses have been proposed to explain the unusual pathogenesis of TSEs including the “protein only” hypothesis which contends that PrP^{Sc} alone is necessary and sufficient for disease transmission by converting PrP^{C} into its disease-associated conformation. However, with the exception of mutagenesis, this hypothesis does not provide a satisfactory explanation for the spontaneous origin of TSEs without the introduction of “Protein X”, an additional hypothetical chaperone-like molecule, into the PrP^{C} to PrP^{Sc} transformation process. As mentioned above, the function of nucleic acids (NA), in TSEs have been studied as source of encoded genetic information and as modulators of PrP structure facilitating prion disease development. Highly structured RNAs were shown to interact with PK-sensitive PrP (PrPSen) to form a stable complex in which RNA became nuclease-resistant. Addition of serum converted PrPSen to PrPRes in the complex demonstrating that PrPRes can be generated in vitro under physiological conditions in the presence of NA and non-NA chaperones and in the absence of seeded PrP^{Sc}. Thus, RNA and a component from serum termed Q-Factor may fulfill the role of “Protein X”. In one embodiment of the invention, this Q-Factor is identified as α-2 macroglobulin (α2M) and is used as one of the chaperones of the disclosed TRIPARTITE system.

In other embodiments, the TRIPARTITE system, described and claimed herein, also sheds light on the causative mechanisms that underlie non-prion related protein misfolding disorders that include the amyloid diseases such as Alzheimer's and Parkinson's, immunoglobulin-light-chain and reactive amyloid diseases, familial amyloid polyneuropathies and various systemic amyloidoses. Thus, the TRIPARTITE system is also useful as a model for amyloid diseases and can be reconfigured as a drug screening procedure for these diseases.

For example, the ubiquitous nature of the TRIPARTITE system and known commonality between Tau protein and prion protein (see Table below) allowed us to modify a prion protein model system into a system that represents human, Alzheimer's disease.

TABLE 1
Similarities Between Prion Protein and Tau Protein

| | | Prion Protein | |
|---|---|---|---|
| | | (Scrapie - from | Tau Protein |
| | | Sheep) | (Alzheimer's) |
| 1. | Aggregates in full length | yes | yes |
| 2. | Dependence on chaperones | yes | yes |
| 3. | Detachment from natural | yes | yes |
| | substrate | | |
| 4. | Insolubility | yes | yes |
| 5. | PK-resistance | yes | yes |
| 6. | Molecular composition/ | similar to Tau | similar to PrP |
| | repeats | | |
| 7. | Accumulation of beta- | yes | yes |
| | sheets | | |
| 8. | Affinity to polyaionic | yes | yes |
| | metabolites | | |
| 9. | RNA as a chaperone | yes | yes |
| 10. | Protein/RNA ratio | 6/1 | 4/1 |

Data obtained using Tau protein demonstrated that the chaperones of the PrP model system misfold Tau protein into an isoform with amyloid characteristics (see FIG. 49). Thus, in certain embodiments of the invention the TRIPARTITE model can be used to discover treatments for Alzheimer's disease. The general approach for moving to a TRIPARTITE-AD system, or any other TRIPARTITE system directed to diseases associated with the misfolding of proteins, is to identify both NA and non-NA chaperones for the protein that are either facilitators or inhibitors of the protein misfolding process.

Using BSE as a model system, the present invention can also isolate and identify molecular chaperones that cause the conversion of the benign “normal” prion protein to an abnormal (misfolded) isoform, thus resembling infectious prion protein modifications. Thus, an embodiment of the invention encompasses an in vitro system (“TRIPARTITE System”) that models the transformation of PrP from a proteinase K (pK) sensitive isoform to its alternative, pK resistant, isoform. Experimental data indicate that the TRIPARTITE system models essential events of the in vivo refolding cascade that occur during PrP conversion from a normal conformation to a scrapie disease isoform. Thus, this system can serve as a powerful tool for elucidating the underlying pathogenic mechanisms of prion related diseases or as a screening tool for identification of anti-prion compounds.

More importantly, the TRIPARTITE system has shed light on the causative mechanisms that underlie non-prion related human protein misfolding disorders that include the amyloid diseases such as Type II diabetes, Alzheimer's and Parkinson's, immunoglobulin-light-chain and reactive amyloid diseases, familial amyloid polyneuropathies and various systemic amyloidoses. Because TRIPARTITE may also be a model for amyloid diseases, based on the misfolding of Tau protein (one of the protiend involved in the Alzheimer's), the system has the potential to be reconfigured as a drug screening procedure for these diseases as well.

By designing a relevant in vitro model of a misfolded protein disease (TRIPARTITE system) and by constructing a transgenic animal model system, discussed and exemplified below, that retains the essential physical and chemical features of protein misfolding and mimics the human disease process, the current invention provides a novel tool for understanding the amyloid disease etiology. The significance of the TRIPARTITE systems is expected to have far-reaching consequences for both the research and drug development communities. It will provide scientists with the unique and powerful tools required to “see” the molecular mechanisms of protein misfolding and enable pharmaceutical companies to “find” active compounds against molecular targets that can ultimately be turned into effective drugs.

The present invention also pertains to compositions and methods for identifying cellular factors involved in protein agglomeration. One such factor is a nucleic acid (NA) component, discussed in detail above. Another factor is a cellular binding factor (CBF) component. The presence of certain NAs and CBFs in a mixture containing proteins like prion proteins will facilitate the agglomeration of these proteins. This invention provides compositions and methods useful for identifying CBFs involved in this agglomeration process. Further, this invention also pertains to methods for assessing the efficacy of a putative pharmaceutical in preventing protein agglomeration formation. Moreover, a kit is described herein that can be used by practitioners for examining test agents and their ability to prevent protein agglomeration formation.

Thus, the critical basis of the TRIPARTITE system is the combination of three elements: cellular isoform of protein and two facilitators (which mimic the natural disease process). In an embodiment described below, one of the identified facilitators is used to generate transgenic animals and cell cultures, to provide model systems for studying the disease process.

Functional ligands like NA antibodies have potential as a highly specific molecular therapeutic. It has been demonstrated that the interaction of a constructed prion-specific NA antibody with a soluble, proteinase K digestible form of the prion protein makes this protein non-soluble and proteinase K resistant. This interaction of functional ligands can be exploited for identifying additional metabolite(s) and cell component(s) that might represent additional groups of functional ligands with a special interest for future therapeutic purposes related to prion diseases. Specifically, the present inventors demonstrated that this functional interaction is dependent upon at least one other functional ligand, namely a cellular binding factor (CBF). (The present invention encompasses one or more CBFs, however, for convenience and simplicity the singular form of CBF is used throughout unless stated otherwise). It is apparent that the functional ligand CBF, identified with the help of another functional ligand, specifically a prion protein specific NA antibody, is a potential target for therapeutic application. Nucleic acid antibodies can play an important role, not only in identifying new targets for therapeutics, but also as therapeutics themselves. Inactivation of CBF using a modified NA antibody would prevent prion proteins from agglomerating, hence blocking an important part in the overall prion-based disease process.

The major advantages of the TRIPARTITE system are as follows:

1. TRIPARTITE system is a true “test-tube” model of the physiological events that cause protein misfolding to lead to amyloid production and may be “universally” adapted to mimic the misfolding events of many amyloid diseases.

2. In vitro and in vivo validation of the TRIPARTITE system as a model for neuritic plaque formation, the primary etiological event in many neurodegenerative disease disorders such as Alzheimer's disease, has been in Tau protein refolding experiments.

3. The TRIPARTITE system involves the use of molecular chaperones which are necessary for transforming proteins into alternative folded states, providing an important research tool for unraveling the molecular and structural events that cause amyloid formation or that maintains the correctly folded state.

4. The TRIPARTITE system is both simple (containing only three homogeneous and well defined biological compounds) and robust. It is scalable in both size, allowing the synthesis of desired quantities of partially folded intermediates, and in the number of experiments that can be performed using standard micro-titer plate techniques.

5. The TRIPARTITE system is easily adapted to high-throughput micro-titer compound screening techniques used to find lead compounds that can either stabilize the normally folded structure or antagonize the pathway that leads to a misfolded protein.

### BSE Model System

When one incubates non-infectious prion protein (PrPc) with a proprietary RNA (RQ 11+12) and bovine cow serum (BCS), the PrP misfolds and the infectious prion protein (PrPRes) is formed which continues the cascade event and begins to aggregate.

### Identity of “Chaperones”

In an embodiment of the TRIPARTITE system, two chaperone-like compounds, alpha-2-macroglobulin and an RNA sequence (RQ 11+12) are isolated. Both of these facilitate the prion protein misfolding and transformation of PrPc into PrPRes. These two compounds are potential targets for “drugs” themselves or, more practically, targets for drug discovery. Thus, the TRIPARTITE system offers the opportunity to find multiple targets for potential drug discovery.

### Cell Culture Model System for Alzheimer's Disease

Embodiments of the subject invention include a cell model system that allows for accelerated drug development programs in Alzheimer's disease, as a supplement to primary screening, assessment of hits, optimization of potential lead compounds, and for in vitro toxicology. The development of anti-Alzheimer's drugs would use the cell culture based model system of the present invention for initial assessment of compounds, discovered using TRIPATITE system, prior animal model studies. Therefore, in order to make the widest and most comprehensive models systems, disclosed herein is a double transgenic cell line. The cell line will express human Tau protein and the RNA facilitator to create a cell culture model system for Alzheimer's disease.

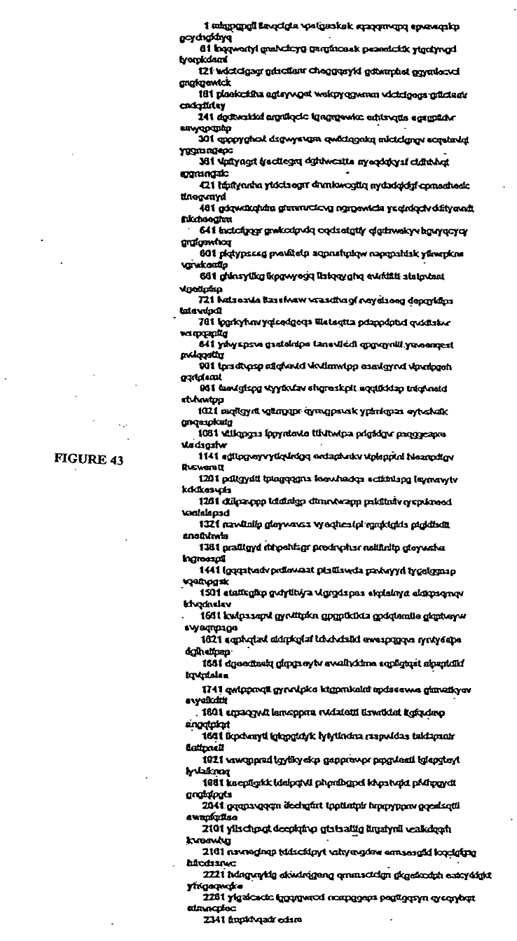

It appears that this previously unknown cellular component, CBF, participates in prion-based disease etiology, development, and progression. Data indicates that one particular CBF is a high molecular weight component, for example, fibronectin or a member of the family of lipoproteins. See FIG. 43 (SEQ ID NO. 7, accession no. NP 002017, also see, Komblihtt, A. R., et al. PNAS, USA 1983,80(11):3218-22). The discovery of the interaction between the two functional ligands, i.e., CBF and NA prion-specific antibody with a prion protein opens a new direction for managing devastating diseases that involve this triumphant.

The discovery of CBF has far reaching consequences. Breeding of animals without a CBF or modified CBF will produce a breed of cows that potentially could never develop mad cow disease. On the other hand, inactivation of CBF by specially designed therapeutics should also reduce the probability of animals and humans to develop prion-based diseases.

One embodiment of the present invention describes a method for identifying the presence or absence of one or more CBFs in a sample matrix. This method comprises taking an aliquot of the sample and adding a predetermined NA antibody, such as RQ11+12 RNA, or a functional fragment thereof to the sample. (A functional fragment refers to a NA fragment that functions, e.g., binds to and interacts with a target protein as the native parent NAY but has a truncated or modified nucleotide sequence that does not affect its binding or interaction property.) To this admixture is added a predetermined protein. This protein is a known protein that forms agglomeration complexes under suitable conditions and may have at least two conformations. The first conformation of the protein is of the active protein. Preferably, the free-form of the protein is in its first conformation or active form. The second conformation is the conformation assumed by the protein in an inactive form. Preferably, in the agglomeration complex, this second, inactive conformation predominates over the first conformation. An example of such a protein is a prion protein. The admixture is then incubated under conditions suitable to allow for the agglomeration of proteins, like those conditions outlined in PCT/US02/16922. In one aspect of the invention, the agglomeration complex comprises a protein component (“A”), a cellular binding factor (“B”), and a NA antibody (“C”), wherein the complex is represented by the formula: [Ax By C,], wherein x, y, and z are integers each independently having a value from one (1) to infinity (∞). The order of constituents in the formula does not represent any particular order in the actual complex. The NA antibody (“C”) is a NA obtained from a nucleotide library comprising NA antibodies including, but not limited to, SEQ ID NOS 1-6. In one aspect, the binding forces holding the complex intact are a collection of non-covalent bonds including, but not limited to, hydrophobic, ionic, Van der Waals, hydrogen bonds, and a combination thereof.

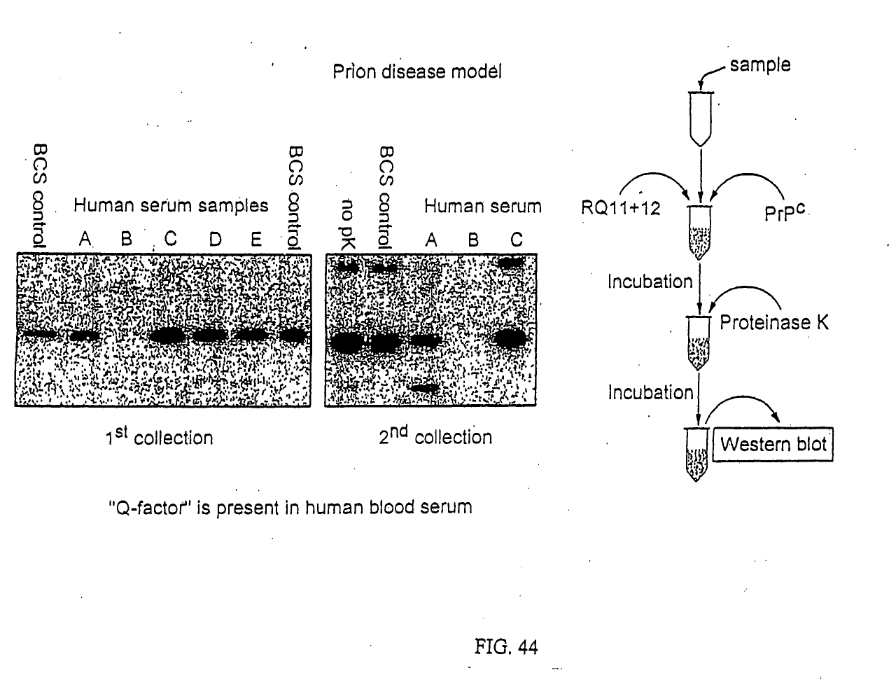

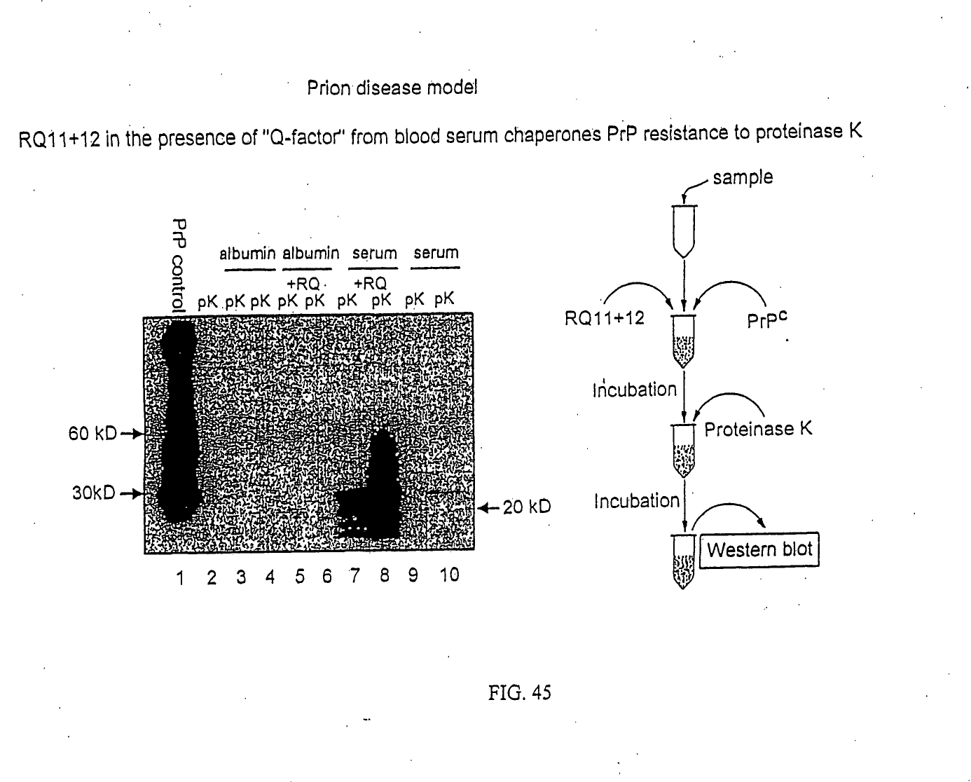

In one embodiment of the invention a method for detecting agglomeration comprises augmenting the method articulated above with protease K, i.e., following incubation of sample matrix, NA and protein, protease K is added to the mixture, and examining protein resistance to digestion. In the absence of a NA antibody and CBF, certain proteins like prion proteins are free and soluble, as a consequence, the proteins are readily digested by proteinase K. In the presence of an appropriate NA antibody and CBF, the prion protein is non-soluble and resistant to proteinase K. These prion proteins present in the mixture should agglomerate and can be detected. The presence of prion protein fragments following protease K treatment is examined by, for example, gel electrophoresis, together with Western blot analysis. If the prion protein is bound in an agglomeration complex, then it is not susceptible to digestion by protease K, hence upon examination there will be a negative, or minimal, finding of prion protein fragments thereby indicating the presence of one or more CBFs in the original sample matrix. FIGS. 44 and 45 depict typical results of such an experiment.

The CBF itself can be isolated from an agglomeration complex. A protein complex, of the type described herein, presumably comprises one or more NA antibodies, a protein target (such as prion protein) and one or more CBFs. The complex can be disrupted using, for example, chaotropic agents. In an experimental setting, the NA antibody and target protein will be known, thus the third major element will be the CBF. The chaotropic mixture can then be subjected to chromatography and/or gel electrophoresis employing known standards for the NA and target protein. The chromatography peak and/or electrophoresis band of unknown origin can then be isolated and further examined. In actual practice, one or more chromatographic procedures may be required to isolate and purify the CBF(s). The isolate, i.e., the putative CBF, can then be analyzed for its ability to confer, for example, proteinase K resistance upon a target protein. Thus, confirming its identity as a CBF.

In one embodiment of the present invention, a method for examining the efficacy of a pharmaceutical agent with respect to its ability to inhibit protein agglomeration is described. Sensitivity to protein digestion by a prion protein is indicative of its proclivity toward participating in protein agglomeration. It is well appreciated in the art that a protein's 3-dimensional conformation can affect the protein's susceptibility to protein digestion as well as its participation if complex formation. Insensitivity toward protein digestion using, for example, protease K, suggests that a prion protein's conformation has change in such a manner so as to facilitate protein agglomeration. Conversely, if the prion protein is susceptible to protein digestion, then it must be free and soluble and therefore, not participating in an agglomeration complex. This understanding provides the basis of an assay system that can be used to assess the efficacy of a putative pharmaceutical agent in inhibiting protein agglomeration.

Thus, an embodiment of the present invention is a method comprising forming an admixture having a CBF together with a NA antibody, a prion protein, and a test pharmaceutical agent. A protease is added to this admixture, such as protease K, under conditions suitable for protein digestion. After a suitable period of time, an aliquot is retrieved from the admixture reaction vessel and the state of the native prion protein is examined. If upon examination protein digestion products are detected, then the pharmaceutical test agent is effective in preventing the soluble prion protein from converting into its non-soluble, agglomerating conformation. Thus, the test agent is effective in preventing the formation of an agglomeration complex. However, should there be little if any digestion product upon examination, then the agent is not efficacious in preventing the formation of an agglomeration complex. Moreover, an agglomeration complex should be evident upon further analysis using, for example, an electron microscope.

The present invention pertains to an assay kit. This kit can be used to assess a test agent's ability to prevent protein agglomeration. The kit comprises a NA antibody, a CBFs, and a protein. The kit can additionally comprise protease K. The NA antibody can be selected from the group consisting of SEQ ID NOS. 1-6. The CBF used in this kit can be alpha-2-macroglobulin, fibronectin (e.g., SEQ ID 7) or a member of the lipoprotein family. In a particular aspect, the protein of the kit is a prion protein. Preferably, this kit is used in conjunction with the methods described herein.

### Validation Experiments

Below are descriptions of validation studies performed on the TRIPARTITE system:

1. Test of Inhibitors and Potential Anti-Alzheimer's Drugs

TRIPARTITE system was validated with several known compounds that have been shown to inhibit aggregation of prion protein in cell culture and animal models. Among them were well-characterized heparin and tetracycline that demonstrated a strong dose-dependant inhibition of PrP aggregation in TRIPARTITE system. Chlorpromazine has been considered as a potential anti-prion compound and was used as an experimental drug on a CJD patient. The Company found that this compound inhibited PrP aggregation in a dose-dependant mode as well. Additionally, the TRIPARTITE system was challenged with two experimental anti-Alzheimer's compounds, 3-nitrophenol and 2,3-dinitrophenol. These anti-AD compounds also demonstrated inhibitory effect on PrP aggregation in TRIPARTITE system.

2. Chemical Validation

In addition to an RNA chaperone, a protein chaperone is present in total plasma and blood serum that facilitate PrP transformation. Subsequently, in one embodiment, this component was purified and identified as alpha-2-macroglobulin. To make TRIPARTITE more friendly for use and experimentation, it was further optimized by synthesizing fluorescent-labeled PrP that allowed us to monitor PrP transformation with great speed and ease. The optimized system was useful for detecting the transformation of PrP originating from different recombinant PrP species, including human, cow, mouse and hamster.

3. Gross Morphology Studies of PrP Aggregates

The studies of PrP aggregates obtained in TRIPARTITE system under light microscope lead to the conclusion that they resemble classical amyloid aggregates. The detailed microscopic analysis of these aggregates provide a preliminary glimpse of their morphology that resembles the PrP aggregates that are usually observed in the brains of scrapie-infected animals and CJD patients. The nature of these aggregates was examined using Congo red dye. Under polarized UV illumination, the amyloid aggregates produced a highly specific bright green color, which unequivocally indicates that they are amyloids in nature.

4. Molecular Structure Studies

The amyloid nature of PrP aggregates generated in the TRIPARTITE system was further confirmed by studies of their molecular architectures using electron microscopy. Here, a variety of sub-microscopic structures were observed, including tangles, fibrils and filaments—structures that are characteristic of naturally occurring, PrP amyloid aggregates in scrapie animals. A precise physical methodology that elucidate molecular structure of proteins and allow to differentiate between alpha- and beta-structures, so-called FTIR analysis, demonstrated that the benign PrP isoforms with characteristic alpha-structures changed in conformation and became rich with beta-structures that are specific for amyloid PrP isoform after binding to RQ11+12. FTIR analysis of PrP interaction with RQ11+12 further confirmed the concept that the TRIPARTITE system represents a specific portion of PrP misfolding cascade.

The features and other details of the invention will now be more particularly described and pointed out in the examples. It will be understood that the particular embodiments of the invention are shown by way of illustration and not as limitations of the invention. The principle features of this invention can be employed in various embodiments without departing from the spirit and scope of the invention.

In addition to the TRIPARTITE system and the cell culture system, the present invention provides transgenic animal models (animals in which a modification such as gene insertion is done to study disease and/or impact of drugs on disease) for amyloid diseases. The development of such models is novel and is pioneering technology in the field of misfolded proteins.

Embodiments of the present invention have particular utility for identifying compositions that participate in protein agglomeration disease processes. A method has the steps of providing an animal that over expresses an untranslated, non-coding RNA. And, the method comprises the step of placing a composition in said animal and evaluating the animal for at least one characteristic of agglomeration disease. A characteristic of agglomeration disease is preferably selected from the group comprising aggregate formation and behavior abnormalities. Aggregate formation associated with brain disorders is found in association with neurons. Aggregate formation associated with other disorders may be found in arteries or other affected organ systems. Behavior abnormalities include learning and memory impairment. In Drosophila melanogaster systems, behavior abnormalities include courtship behaviors, song or chirping patterns, decreased memory capacity and locomotion impairments. In mammalian systems, including mice, behavior abnormalities are decreased memory, decreased learning capability and locomotion impairments.

Embodiments of the present invention will be described in detail with respect to Drosophila melanogaster and mouse systems with the understanding that other animal and plant systems can be readily employed as well. In one embodiment the animal is selected from the group of animals comprising Chordata, Arthropoda, Nematodata and Mullusca. A preferred animal from the Arthropoda phylum is selected from the subphylum Insecta, and, is, most preferably, Drosophila melanogaster. A preferred animal from the Chordata phylum is a mammal and, a preferred mammal is a mouse, rat or primate.

As used herein, “placing” means causing the composition to be placed in contact, inserted or ingested the composition into the animal or causing the composition to be injected or inhaled into the animal or other means. Injection of a composition can be done intramuscularly, intravenously, into the body cavity or otherwise.

The RNA that is expressed is preferably an RNA of twenty to 1,000 nucleotides and most preferably, approximately, 100 to 500 nucleotides. The RNA is preferably complex in the sense of having at least one section forming a hairpin structure and having at least one loop structure. A preferred RNA has sequences derived from retrovirus. As used herein, the term “non-coding” means that the RNA does not code for a complete protein and transcription products related to such RNA can not be identified in the cell in which such RNA is placed. For example, without limitation one preferred RNA has at least one section of ten to five hundred nucleotides in a sequence derived from a retrovirus transposable element or transposon or other untranslated portion of the genome. One preferred, retroviral sequence is derived from HIV. Preferably, the expressed untranslated RNA sequence has at least one section of ten to five hundred nucleotides that are recognized by an RNA dependent RNA polymerase. As used herein, the term “recognized” means that the section allows the RNA to be replicated by the RNA dependent RNA polymerase under conditions in which the enzyme normally replicates RNA templates.

A preferred expressed untranslated RNA corresponds to RQ 11+12. As used herein, the term “corresponds” means having substantially the same nucleotide sequence as, or opposite pairing relationship as in the DNA encoding for such RNA. Preferably, the expressed RNA has twenty nucleotides in a sequence that substantially corresponds to the sequence of RQ 11+12.

Embodiments of the present invention feature a non-naturally occurring animal cell transformed to express a non-coding RNA. One further embodiment comprises and method of making a transformed animal that expresses a non-coding RNA the method comprises the steps of placing a non-coding RNA under the control of a promoter in an animal cell and growing an animal from the cell. The method further comprises the step of placing the animal or cell having the non-coding RNA under conditions in which the RNA is transcribed.

A preferred inducible promoter for use with lower forms of animals such as Drosophila melanogaster is a heat shock promoter. A preferred inducible promoter for use with higher animals, such as mouse systems comprise of antibiotics or heat shock inducible promoter.

### Drosophila melanogaster

The subject invention provides a novel animal amyloid model system using the fruit fly or Drosophila. The Drosophila is an animal species that has been exhaustively studied by biologists over the years and, with such knowledge, the usefulness of these animals as model systems is dramatically enhanced.

Using the RNA ligand RQ 11+12 (see SEQ ID NO: 1, above), a transgenic fruit fly was generated. More specifically, a method for constructing transgenic animals was developed through introduction of functional RNAs into the Drosophila. This method is different from known, traditional methods for construction of transgenic animals through introduction and expression of appropriate proteins.

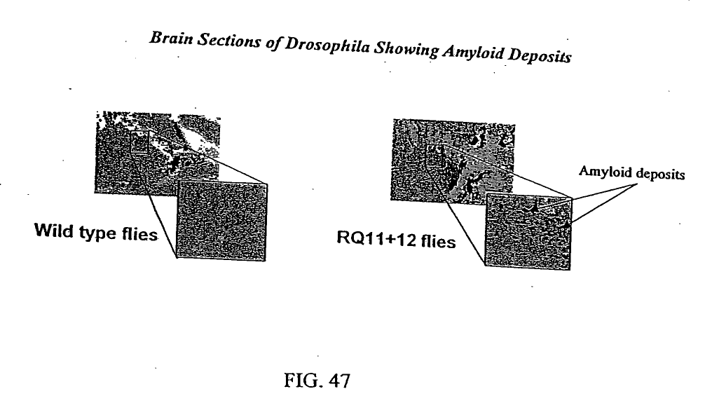

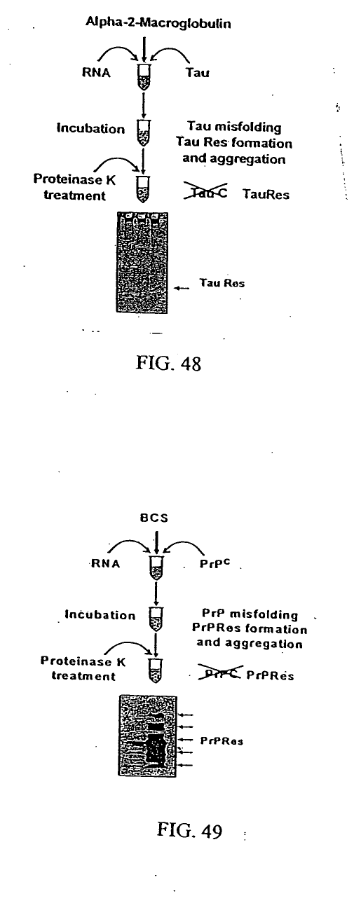

Induced production of RQ11+12 RNA in the transgenic flies leads to massive accumulation of amyloid deposits in their brains (see FIG. 47). The accumulation of amyloid deposits resulted in an impairment of the flies' memory capacity, learning ability and motor activity. In general terms, the generated transgenic flies are unique and novel models for neurological disorders that affect both central cognitive and peripheral motor nerve systems.

The constructed transgenic Drosophila can be optimized into a powerful system to study molecular and structural biology of human neurological disorders; identify new compounds that will enhance memory capacities, increase learning abilities, and interfere with the origin and progression of human neurological disorders; validate therapeutic potential of known target compounds; identify natural chaperones that will hinder neurological condition development.

One embodiment of the present invention is directed to a model system for agglomeration disease comprising an animal that expresses an untranslated, non-coding RNA. Animals are normally selected for their similarity to humans and/or the ease of tranforming such animal, and the speed in which the animal matures. Common animal systems are selected from the group of animals comprising Chordata, Arthropoda, Nematodata and Mullusca. A particularly preferred animal selected from the Chordata Phylum is a mouse, rat and several species of primates. A particularly preferred animal selected from the Nematodata Phylum is C. elegans. A particularly preferred animal selected from the Arthropoda phylum is an insect, and, most preferably, Drosophila melanogaster, the common fruit fly. The common fruit fly is particularly useful because it matures rapidly and is relatively easy to transform, control and maintain.

The untranslated RNA that is expressed is preferably an RNA of twenty to 1,000 nucleotides and most preferably, approximately, 100 to 500 nucleotides. The RNA is preferably complex in the sense of having at least one section forming a hairpin structure and having at least one loop structure. As used herein, the term “hairpin” refers to a structure of RNA in which the RNA molecule folds onto itself and exhibits affinity for complementary sequences within the molecule. A “loop” is a section of the RNA molecule between to sections which exhibit affinity for each other. That is, the loop is a section of the RNA between two sections form a single hairpin structure.

These complex structures are found in naturally occurring RNA of viral origin. A preferred RNA has sequences derived from retrovirus. However, it not necessary for the entire retroviral sequence to be used. As used herein, the term “non-coding” means that the RNA does not code for a complete protein and translation products related to such RNA can not be identified in the cell in which such RNA is placed.

One preferred RNA has at least one section of ten to five hundred nucleotides in a sequence derived from a retrovirus. One preferred, retroviral sequence is derived from HIV.

Preferably, the expressed untranslated RNA sequence has at least one section of ten to five hundred nucleotides that are recognized by an RNA dependent RNA polymerase. As used herein, the term “recognized” means that the section allows the RNA to be replicated by the RNA dependent RNA polymerase under conditions in which the enzyme normally replicates RNA templates. A preferred expressed untranslated RNA corresponds to RQ 11+12. As used herein, the term “corresponds” means having substantially the same nucleotide sequence as, or opposite pairing relationship as in the DNA encoding for such RNA. Preferably, the over-expressed RNA has twenty nucleotides in a sequence that substantially corresponds to the sequence of RQ 11+12. The RNA RQ 11+12 is a Q-beta replicase template containing HIV-Rev binding element (underlined in the sequence presented below), and the Sarcin/Ricin cleavage domain (italicized in the sequence presented below). The sequence for the RNA RQ 11+12 is set forth in Sequence No 1.

(SEQ ID NO: 1)

5′
GGGGUUUCCAACCGGAAUUUGAGGGAUGCCUAGGCAUCCCCCGUGCGUCC
CUUUACGAGGGAUUGUCGACUCUAGUCGACGUCUGGGCGAAAAAUGUACGA
GAGGACCUUUUCGGUACAGACGGUACCUGAGGGAUGCCUAGGCAUCCCCGC
GCGCCGGUUUCGGACCUCCAGUGCGUGUUACCGCACUGUCGACCC-3′

The non-coding RNA and promoter are preferably incorporated in a vector. A preferred vector for placing such RNA in Drosophila melanogaster is a Casper expression vector. The Casper expression vector is based on the well-characterized pUC vector.

The vector is preferably placed in the cell by microinjection techniques. Normally, many cells are transformed by microinjection and those that successfully receive the vector replicate and develop further. For example, without limitation, Drosophila melanogaster cells which receive the vector will develop into larva, pre-pupae and finally adult flies.

The expression of the non-coding RNA is controlled by the temperature at which the transformed Drosophila melanogaster is maintained. Subjecting the transformed insect to a temperature of approximately 37 degrees centigrade causes the expression of the non-coding RNA.

The transformed animal is preferably allowed to mature. Preferably, the animal is evaluated with respect to suitable controls to study the effects of the non-coding RNA. The expression of non-coding RNA causes pathologies and behavior changes that are of the same nature and kind as exhibited by animals with agglomeration disease. The neurological abnormalities observed coincided with a massive deposition of aggregates in the brain of affected flies. Similar to aggregates observed with PrP in the TRIPARTITE system, the aggregates observed in Drosophila were Congo red-positive, indicating that they were amyloidogenic in nature.

The transformed animal can be used to evaluate potential treatments and drug candidates. The ability of a composition to alter the progression of pathology and behavior changes over time may suggest a potential drug candidate.

### The RQ Mouse

Current transgenic mouse models of Alzheimer's, Parkinson's and other related disorders are genetically programmed to express corresponding human proteins and to simulate motor deficits and brain alterations found in these diseases. They were used in studies of human neurological disorders as well as for drug discoveries and validation. However, an important characteristic of traditional transgenic animal models is that the disease is a result of over-expression of the human protein. The transgenic animal model described herein develops a neurological condition in a more natural way, without introduction of the foreigner protein and without over-expression of this protein. Expression of RQ11+12 RNA is induced and the effect of the synthesized RNA on aggregate formation in the brain and other mouse tissues can be validated using standard cytological, molecular biology and biochemical methods.

The success of the Drosophila model system was exploited by using the same technology and techniques to develop a murine (mouse) animal model system of Alzheimer's disease.

Transgenic mice are produced by injecting a construct with RQ11+12 RNA under regulated promoter into one-cell embryos followed by implantation into foster mice. A similar procedure is commonly performed with DNA that encodes the desirable protein but can be performed with an appropriate construct that expressed non-coding RNA, such as RQ11+12 as well. After approximately 3 weeks, a standard gestation period of a mouse, pups will be ready for weaning. At 3 weeks of age, pups will be ready for tail biopsies for DNA analysis of transgene integration.

In certain embodiments of the invention these animal models are used to validate drugs discovered using the TRIPARTITE system.

Embodiments of the present invention will be described with respect to a kit for performing a separation of an agglomeration protein from a biological fluid and methods of performing such a separation with the understanding that the description is directed to the preferred embodiment. Individuals skilled in the art will recognize that features and elements of the invention are capable of alteration and modification. Thus, the present discussion should not be considered as limiting.

The kit has the following major elements; an immobilized phase contained in vial, reagents contained in a second vial, packaging and instructions.

The kit is for separating an agglomeration protein from a solution potentially having the agglomeration protein in a mixture of biological materials. The first vial contains an immobilized phase for combining with an aqueous solution to form a wetted immobilized phase. The immobilized phase has a nucleic acid selected from the group of poly A, poly U, poly G, or poly C and poly dA, poly dG, poly dC and poly dT of ten to 200 nucleotides in length. The immobilized phase is selected from the group comprising membranes, frits, dispersable particles and packed beds of particles. Preferred particles are silica, alumina, cellulose, latex and iron oxide. A cellulose matrix having 25 nucleotides of poly dT is preferred and available from several vendors. Means for bonding oligonucleotides to particles and membranes are known in the art.

For this discussion, the solution has or potentially has an agglomeration protein. In the presence of the agglomeration protein, the nucleic acid forms a binding product, under binding conditions, with the agglomeration protein which binding product is bound to the immobilized phase. The reagent vial contains reagents comprising buffers and the like for placing the solution under binding conditions. These binding conditions are normal physiological conditions of salt concentration and acidity. Such normal physiological conditions are known in the art.

Instructions inform the user regarding aspects of the method of forming a wetted immobilized phase, imposing binding conditions on the wetted immobilized phase to form an binding product in the presence of the agglomeration protein and separating the immobilized phase from the aqueous solution to form a separation product.

In the presence of the agglomeration protein, the separation product includes a binding product. The removal of the binding product reduces the concentration of the binding product, which includes agglomeration proteins. Thus embodiments of the present invention can be used to remove agglomeration proteins from sources of biological tissues, such as blood.

This binding product is concentrated on the immobilized phase and is more readily detected over background. One preferred embodiment phase further comprises instruction for detecting the presence or absence of the binding product as an indication of the presence or absence of an agglomeration protein. Preferably, the kit further comprises an antibody or a nucleic acid having specific affinity to the agglomeration protein held in a suitable vial. Antibodies and nucleic acid ligands having specific affinity for several agglomeration proteins are known in the art.

Embodiments of the present invention with respect to the methods of the present invention will now be described in the context of the Examples that follow.

### EXAMPLES

### Example 1

RNA Ligands

The ability to amplify PrP-Amp was addressed using Q-Amp. Under conditions suitable for amplification, MNV and PrP-Amp were independently subjected to Q-Amp. FIG. 7 presents data demonstrating the along with MNV, PrP-Amp can be amplified by Q-Amp replicase. An example of a suitable protocol for Q-Amp amplification provides a 80 mM Tris-HCl buffer at pH 7.5 together with 20 mM MgCl₂, 2 mM dithiothreitol, 200 μM rNTPs, and 200 nM replicase. In a 20 μL reaction, RNA is added and incubated for thirty (30) minutes at 37° C. Under some circumstances, 1 μL [α-P³²] CTP (3000 Ci/mmol) is added to the reaction in order to label the nascent RNAs. Alternatively, detection of amplification products is accomplished by subjecting them to polyacrylamide gel electrophoresis followed by staining the nucleic acids in the gel using, for example, SYBR Gold (from Molecular Probes, Eugene, Oreg.).

The binding specificity of PrP-Amp was next examined and compared to MNV.

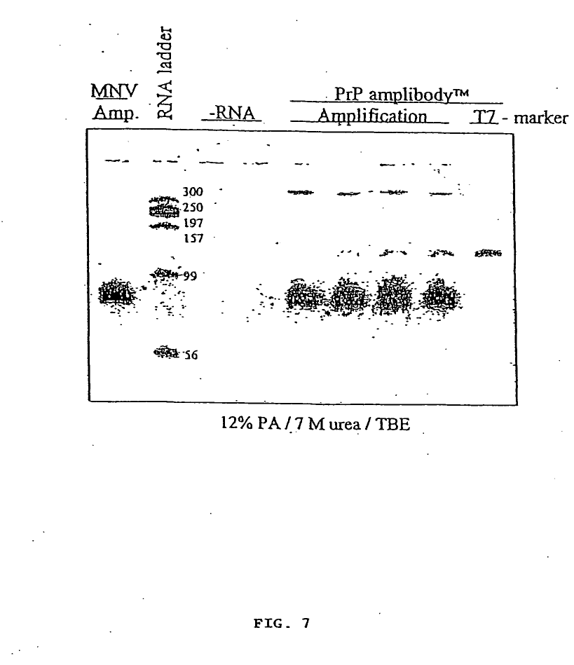

FIG. 8a shows the binding data. To fully appreciate this figure, it must be realized the reason for using two membranes in this experiment. See Weiss, S., et al., J Virol November 1997, 71(11):8790-8797; the entire teachings of which are incorporated herein by reference. Protein and bound RNA will bind to the PVDF membrane. The bound RNA refers to that RNA that is bound to a protein. Free or unbound RNA will pass through the PVDF membrane and bind to the nylon membrane. Therefore, if the RNA is labeled and not bound to protein, then it will pass through the PVDF membrane and a signal will be generated from the nylon membrane where the free-labeled RNA is bound. Conversely, should the labeled RNA bind to a protein, then a signal will be generated from the PVDF membrane. With respect to the PrP-Amp, it can be observed that two signals are visible on the PVDF membrane indicating that this amplibody binds to the prion protein, moreover the presence of tRNA does not affect this binding. Unlike PrP-Amp, binding of MNV to the prion protein is affected by the presence of tRNA. In the presence of tRNA, labeled MNV RNA passes through the PVDF membrane passing onto the nylon membrane indicating the lack of binding to the prion protein in the presence of tRNA.

FIG. 8b presents the same data illustrated in FIG. 8a the only difference is that FIG. 8b presents it in a graphical manner. The binding dissociation constant (Kd) presented in Table 2 quantitatively demonstrates that (a) PrP-Amp's binding to the prion protein is independent of the presence of tRNA, whereas, the binding of MNV is drastically reduced in the presence of tRNA, and (b) PrP-Amp has a higher affinity for the prion protein.

The specific mechanisms of initiation and progression of prion fibrillation are not well understood. Previous studies have tried to determine if RNA or DNA play a genetic role in the transmission of prion disease (Akowitz et. al. 1994 NAR 22(6): 1101-07; Nandi and Leclerc, 1999 Arch. Virol. 144(9):1751-1763; Cordeiro et. al., 2001 J. Biol. Chem. 276(52):49400-49409; Narang 1998 Res. Virol. 149(6):375-82; Narang 2002 Exp. Biol. Med. 227(1):4-19; the entire teachings of which are incorporated herein by reference). In this study, the inventors focused on the physical interactions and effects of RNA binding to hrPrP, not the genetics of prion transmission. For this, they used small RNAs of defined sequence to characterize the RNA-binding activities of recombinant human PrP (hrPrP) in vitro. The results obtained demonstrate that hrPrP has high affinity for small RNA species that resides in amino acids 23 to 90 (the N-terminus). The extent of complex formation between PrP and RNA is demonstrated by protection of the RNA species from degradation by RNaseA. They also identified a class of RNAs that tightly bind to hrPrP in the presence of a vast excess of non-specific competitor RNAs. RQ11+12, a newly described RNA that belongs to this class, also showed the ability to bind specifically to endogenous PrP in mouse brain homogenates and generate RNA-PrP complexes. The transcription reagents used for this study (17 RNA polymerase, RNase inhibitor, rNTPs, buffers) were either from Ambion (Austin, Tex.) or MBI Fermentas (Hanover, Md.). Ultra-pure BSA and Nuc-Away spin columns was also from Ambion while Schleicher and Scheull 0.45 mu·M BA85 nitrocellulose membranes were supplied by VWR (Bridgeport, N.J.). Perkin Elmer (Boston, Mass.) supplied the PVDF and Nylon membranes as well as all radioisotopes. The nucleic acid intercalating dyes RiboGreen and SYBR Gold, used for analysis and RNA quantitation were from Molecular Probes (Eugene, Oreg.). General salts, buffers and electrophoresis products were supplied by VWR and Sigma (St. Louis, Mo.). Recombinant human PrP.sup.23-231, PrP.sup.23-144, PrP⁹⁰⁻²³¹ were kindly provided by Man-Sun Sy, Case Western University.) and were purified to homogeneity from Escherichia coli as glutathione S-transferase-tagged fusions (with removal of glutathione S-transferase by thrombin cleavage) as described previously (20). Brain homogenates (10%, w/v) from wild-type mice were prepared as follows. Whole brains (Psychogenics, New York, N.Y., U.S.A.) were homogenized in 9 vol. of PBS supplemented with 0.5% Nonidet P-40, 0.5% deoxycholic acid and protease inhibitor cocktail (for use with mammalian cell and tissue extracts; Sigma). After centrifugation (12000 g for 30 min) the supernatants were aliquoted and stored at −70° C. Protein concentration was measured by Bradford assay, using Pierce reagents.

Homogenates were prepared from whole mouse brains. Ten percent (wt/vol) brain homogenates from wildtype mice and PrP knockout mice (PrP^{0/0}) were prepared as follows. Brains were homogenized in nine volumes of PBS supplemented with 0.5% Nonidet P-40, 0.5% deoxycholic acid and a cocktail of protease inhibitors (Sigma). After centrifugation (12,000×g for 30 minutes), the supernatants were aliquoted and stored at −70.degree. C. The protein concentration was measured by Bradford assay, using Pierce reagents supplied by VWR.

Construction of RNAs:

RNAs were synthesized using T7 RNA polymerase and PCR-generated DNA templates. PCR templates were made from either sequenced plasmids or synthetic oligonucleotides using primers that add the T7 polymerase promoter to the 5′ end and define the 3′ end by run-off transcription. All DNA cloning steps used in the preparation of plasmid templates followed standard molecular biology techniques (Sambrook et al., 1989 Molecular Cloning: A Laboratory Manual. Cold Springs Harbor Laboratory Press, Cold Springs Harbor, N.Y.; the entire teaching of which is herein incorporated by reference). DNA cloning typically involved the annealing of synthetic oligonucleotides, followed by their ligation and cloning into pUC19. RNAs internally labeled were made by the incorporation of [α-³²P] GTP or CTP at varying specific activities, dependent upon the application. All of the RNAs used in this study were gel purified from 12% polyacrylamide/7M urea denaturing gels and then the RNA was collected using an IBI V-channel electroelution apparatus. Prior to gel purification, radiolabeled transcription reactions were prepared using Nuc-Away spin columns (Ambion) while unlabelled reactions were prepared by phenol:chloroform extraction, ethanol precipitation and resuspension in TE (10 mM Tris-HCl, pH 7.4, 1 mM EDTA). The concentration of RNA was measured either as a function of isotope incorporation with a BioScan XER-2000 or by a RiboGreen (Molecular Probes) fluorescence-binding assay using a Sequoia-Turner Model 450 flourometer.

The nucleotide sequences and predicted secondary structures of MDV, MNV (Preuss et al., 1997 J. Mol. Biol., October 31, 273(3):600-13; the entire teaching of which is incorporated herein by reference) and AP1 (Weiss et al., 1997 J Virol, November 71(11):8790-8797; the entire teaching of which is incorporated herein by reference) have been reported elsewhere. The following RNAs, shown in the 5′->3′ orientation, are described for the first time:

(1) AP46 (AP1 with one G-quartet removed) (SEQ. ID NO 7)

5′ GGGAAUUUGAGGACGAUGGUAAGUGG 3′;

(2) AP49-(AP1 with one G-quartet replaced) (SEQ ID NO 8)

5′ GGGAAUUUGAGGAACGAUAGGUAAGUGGA 3′;

BS1577 (non-specific RNA; contains the sequence for (3) below;

(3) 1577 region of Bacillus subtilis 23S rRNA; Ash and Collins, 1992) (SEQ ID NO 9)

5′GGCCCCGUAACUUCGGGAGAAGGGGUGCUCUGUUAGGGUGCAAGCCCGA
GAGAGC 3′;

(4) RQT157 (Q-beta replicase vector RNA) (SEQ ID NO 10)

5′
GGGGUUUCGAACCGGAAUUUGAGGGAUGCCUAGGCAUCCCCCGUGCGUC
CCUUUACGAGGGAUUGUCGACUCUAGAGGAUCCGGUACCUGAGGGAUGCC
UAGGCAUCCCCGCGCGCCGGUUUCGGACCUCCAGUGCGUG
UUACCGCACU
GUCGACCC 3′;

(5) RQ11+12 (Q-beta replicase template containing HIV-Rev binding element (underlined)(Iwai et al., 1992), and the Sarcin/Ricin cleavage domain (italicized)(Endo and Wool, 1982) (SEQ ID NO 1)

5′
GGGGUUUCCAACCGGAAUUUGAGGGAUGCCUAGGCAUCCCCCGUGCGUCC
CUUUACGAGGGAUUGUCGACUCUAGUCGACGUCUGGGCGAAAAAUGUACGA
GAGGACCUUUUCGGUACAGACGGUACCUGAGGGAUGCCUAGGCAUCCCCGC
GCGCCGGUUUCGGACCUCCAGUGCGUGUUACCGCACUGUCGACCC-3′;

(6) MNV:AP1 (AP1 (italicized) in MNV vector RNA) (SEQ ID NO. 4)

5′GGUUCAUAGCCUAUUCGGCU UCGCGCAUGGGAAUUUGAGGGACGAUG
GGGAAGUGGGAGCGCGUUUUAAAGGACCUUU UUCCCUCGCGUAGCUAGC
UACGCGAGGUGACCCCCCGAAGGGGGGUGCCCC 3′;

(7) MNVLO (Q-beta replicase template RNA) (SEQ ID NO. 6)

5′GGGUUCAUAGCCUAUUCGGCUUCGCGCC CCUGGGGUUUGCCUCAGGA
GCGCGUUUUAAAGGACCUUUUUCCCUUGCGUAGCUAGCUACGCGAGGUGA
CCCCCCGAAGGGGGGUGCCCC 3′;

(8) MNVUP (Q-beta replicase template RNA) (SEQ ID NO. 5)

5′GGGUUCAUAGCCUAUUCGGC UUCGCGCCCGUUUAUAAUACUUAGUGA
GCGCGUUUUAAAGGACCUUUUUCCCUCGCGUAGCUAGCUACGCGAGGUGA
CCCCGAAGGGGGGUGCCCC 3′.

Filter Binding Assays:

The binding affinity of hrPrP for various RNA ligands was determined by a two-filter binding method essentially as described (Lochrie et. al., 1997 NAR 25(14):2902-10; Battle and Doudna, 2001 RNA 7:123-32; the teachings of which are incorporated herein by reference). Internally labeled RNA, at a concentration always at least ten-fold lower than protein concentration (typically 50 μM), was incubated with increasing amounts of hrPrP in 20 μL-100 μL reactions in Binding Buffer (10 mM Tris-OAc, pH 7.5, 2 mM MgCl₂, 250 mM NaCl, 1 μg/μL BSA and 2 mM DTT) for 30 minutes at 37° C. When present, tRNA was at a concentration of 10 ng/ul (about. 400 nM). Unlabelled RNAs (tRNA, competitor RNAs) were incubated for 15 minutes with hrPrP prior to the addition of any labeled RNAs. After binding RNA to protein, the reactions were vacuum filtered through a protein binding membrane (PVDF or nitrocellulose) and then a nucleic acid binding membrane (positively-charged Nylon) using a Schleicher and Schuell Minifold II Slot Blot hybridization apparatus. A reaction with no protein was also incubated and filtered in order to determine the amount of RNA that binds to the filter in absence of protein. The filters were imaged using a Molecular Dynamics Storm 820 phoshpor imager and the program ImageQuant. After subtraction of background values, the percent of RNA bound to the PVDF or nitrocellulose filter in a protein-dependent manner was determined by dividing the intensity of the signal from the protein-binding filter by the sum of the intensity of signal from both filters. The percent of input RNA bound was calculated as an average of at least three measurements at each hrPrP concentration. The average standard deviation was always less than 2% of the input RNA. The percentage of RNA bound to protein was plotted against the concentration of the protein using Microsoft Excel. The apparent K.sub.d is defined as the protein concentration at which 50% of maximal RNA binding occurs.

Gel Shift Assays:

The tRNA gel shift experiments were done in Binding Buffer B at 37° C. for 30 minutes. As above, the competitor tRNA was preincubated for 15 minutes in reactions that contain hrPrP. A 2 L of sample buffer (30% gylcerol, 0.01% xylene cyanol/bromophenol blue) was added to each sample prior to loading onto a 6% polyacrylamide/TBE gel run in a 4° C. cabinet. The gels were dried and visualized by autoradiography.

The gel shift experiments using mouse extracts assays were carried out by incubating 50 femtomoles of either ^{32P}-labelled, gel-purified MNV or RQ11+12 in a total volume of 20 μL in retardation buffer (50 mM MOPS, pH 7.4, 5 mM MgCl₂, 50 mM LiCI, 1 mM DTT, 1 μg tRNA and 1 μL bovine serum (Invitrogen) in 0.05% deoxycholic acid and 0.05% NP-40). To the RNA and buffer, either hrPrP, brain extract from wildtype mouse, or brain extract from a PrP^{0/0} knockout mouse was added. Reactions were incubated for 20 minutes at room temperature, and then the samples were loaded on 6% polyacrylamide gel containing 4M urea. Electrophoresis was run for 35 minutes at room temperature in TBE buffer (50 mM Tris-borate, pH 8.3, 1 mM EDTA). The gel was dried and exposed to x-ray film or analyzed by phoshpor image analysis.

RNaseA Protection Assay:

Reactions (20 μL) were incubated for 30 minutes at 37° C. in 10 mM Tris-HCl, pH 7.4, 100 mM NaCl, and 1 mM EDTA. The concentration of RNA was 500 pM where indicated and the concentration of hrPrP was 20 nM when present. After 30 minutes, the concentrations of the reactions were adjusted to 1 pg/·mu·L RNaseA where indicated. After an additional 15 minutes at 37° C., an equal volume of loading buffer was added (94% formamide, 40 mM EDTA, 0.001% bromophenol blue/xylene cyanol), the samples immediately heated to 95° C. for 3 minutes, and then immediately separated on a 6% PA/7M urea/IBE denaturing gel. Image analysis was done using a Molecular Dynamics Strom 820 Phosphorimager.

RNAs Bind to hrPrP with High Affinity

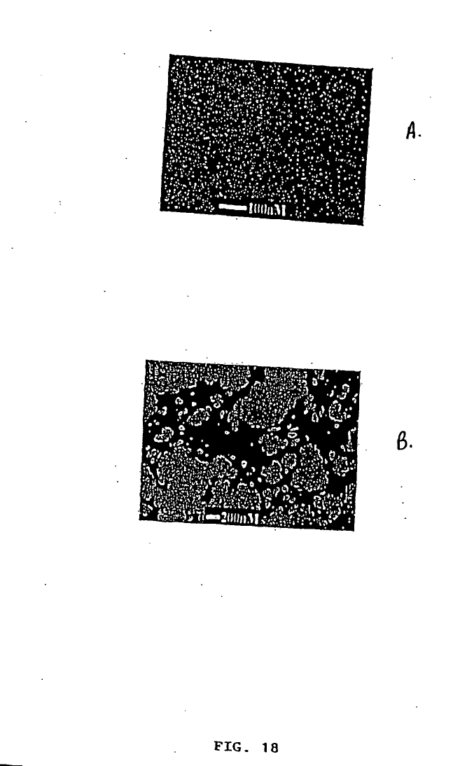

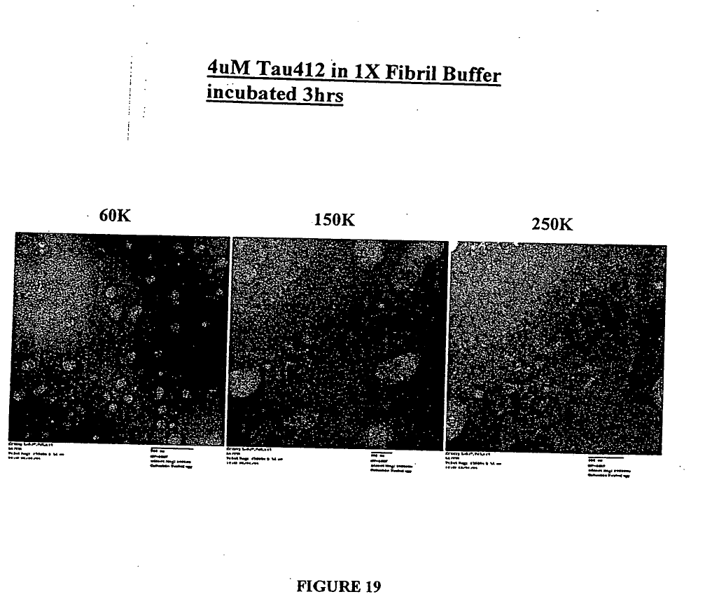

A set of RNAs predicted to have various secondary structures (FIG. 19) were used to study the RNA-binding activities of hrPrP at physiological pH (7.5) in vitro. A filter binding assay was used to determine the apparent dissociation constant (Kd) between each RNA and hrPrP. AP1, an RNA that was isolated from a random library by the SELEX technique on the basis of its affinity towards recombinant PrP, was previously shown to bind to the extreme N-terminus of PrP, within amino acids 23-52. AP1 RNA was used as a positive control because it was selected under similar binding conditions, although its Kd was never reported. The Kd of AP1 for hrPrP was determined to be 5.0 nM (Table 5, -tRNA). Surprisingly, each of the RNAs tested demonstrated a considerable affinity for hrPrP (all Kd<60 nM; Table 5, -tRNA). The tightest binding RNA was MNV:AP 1 with a Kd of 3.8 nM, whereas RQT157 bound the weakest with a Kd of 56 nM. These data demonstrate that hrPrP binds a wide variety of RNAs with high affinity. We term this RNA-binding activity non-specific binding.

tRNA Competition Differentiates Between Binding Specificities

The Kd values for all of the tested RNAs were in a tight range whereas their predicted secondary structures varied considerably, raising the possibility that hrPrP has different RNA-binding specificities. To characterize hrPrP-binding specificity, tRNA was added to block non-specific RNAbinding interactions. In the presence of 10 ng/μl tRNA (˜400 nM), a greater differential in binding affinity was observed, ranging from 12 to 2500 nM (Table 6, +tRNA). The RNAs that were least affected by the presence of tRNA and maintained Kd values below 100 nM include AP1, MNV: AP1 and RQ11+12. The binding of the remaining RNAs is more severely affected by tRNA, in some cases by over two orders of magnitude. The effect of RNA structure on affinity to hrPrP is exemplified by MNVLO and MNVUP, who have a 7-fold variance in their Kd values yet differ by a single stemloop structure (FIG. 19 (b) and FRC; Table 6, +tRNA). The variation in affinity in the presence of non-specific competitor RNA suggests a discriminatory RNA-binding activity for hrPrP represented by RNAs containing AP1 and RQ11+12. This activity, where RNAs can bind to hrPrP with high affinity (Kd<100 nM) in the presence of excess competitor RNAs, we term specific binding.

Small RNAs Bind to hrPrP In Vitro:

Stable complex formation between PrP proteins and nucleic acids has been demonstrated many times both with recombinant and native PrP^{C} in vitro (Nandi 1998, Arch. Virol. 143(7):1251-63; Nandi and Leclerc, 1999, Arch Virol. 144(9):1751-63; Nandi and Sizaret, 2001, Arch Virol. 146:32745; Weiss et al., 1997, J. Virol 71(11):8790-97; the teachings of which are incorporated herein by reference) and PrP^{Sc} in vivo (Merz et al., 1981, Acta Neuropathol (Berl). 54(1):63-74; Murdoch, et al., 1990, Virology 64(4):1477-86; Akowitz et al., 1994, NAR 22(6):1101-07, the teachings of which are incorporated herein by reference). In this study, they further characterized the RNA binding activity of hrPrP with a collection of small, artificial RNAs (56-242 nt). Although not directly relevant to the binding study itself, several of these RNAs have been engineered to be viable templates for exponential amplification by the RNA-dependent RNA polymerase, Q-beta replicase (Preuss et al., 1997, J. Mol. Biol. 273(3):600-13; the teachings of which are incorporated herein by reference).

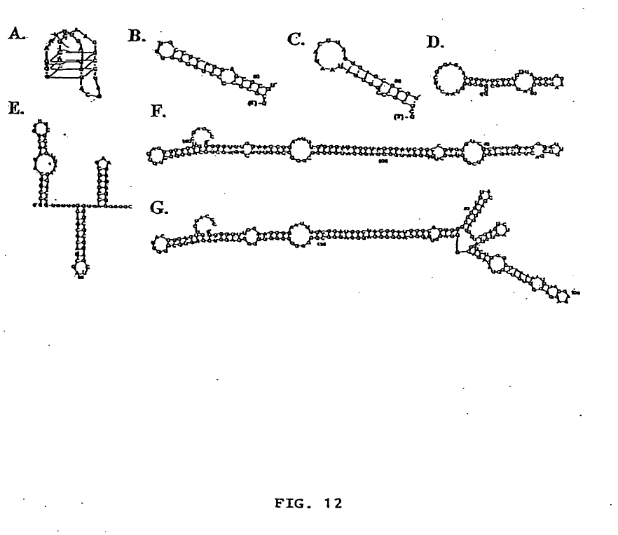

The apparent dissociation constants (Kd) between the RNAs and hrPrP were determined using a two-filter binding assay (Lochrie et. al., 1997, NAR 25(14):2902-10; Battle and Doudna, 2001, RNA 7:123-32; the teachings of which are incorporated herein by reference). Gel purified RNAs and hrPrP were incubated at 37° C. in a high salt buffer (Binding Buffer A; 2 mM MgCl₂, 250 mM NaCl) similar to the one used in the selection of RNA aptamers from a random library with affinity to recombinant Syrian golden hamster PrP^{C} (srPrP) (Weiss et al., 1997). These experiments produced a family of specific PrP-binding RNAs that can be identified by three stacked, G-quartets. In particular, the aptamer AP1 (FIG. 12a) demonstrated the ability to bind specifically to srPrP and the interaction was further localized to the 16 N-terminal amino acids (23-39). The inventors also constructed AP1 and used it as a positive control to evaluate RNA species from our collection. Using their own developed system, the apparent dissociation constant (K.sub.d) of AP1 to hrPrP was determined as 5.0 nM (Table 6).

TABLE 6
(Apparent Kd values for in vitro binding to hrPrP)
Results of filter-binding experiments with isotopically labelled RNA
and hrPrP in binding buffer. The Kd values reported here are means from
at least three separate experiments. The S.D. values were always less than
2% of the calculated Kd, and have therefore been omitted.
Fold effect tRNA = (−tRNA column)/(+tRNA column).

| | Length | | | |
|---|---|---|---|---|
| RNA | (nt) | K_{d}(nM + tRNA | K_{d} (Nm − tRNA | Fold effect |
| MNV:AP1 | 130 | 12 | 3.8 | 3.2 |
| AP1 | 29 | 81 | 5.0 | 16.0 |
| RQ11+12 | 197 | 91 | 26 | 3.5 |
| AP46 | 26 | 100 | 14 | 7.1 |
| AP49 | 29 | 161 | 15 | 11.0 |
| MDV | 244 | 170 | 7.6 | 22.0 |
| RQT157 | 157 | 185 | 56 | 3.3 |
| MNVUP | 118 | 220 | 5.0 | 44.0 |
| MNV | 86 | 1000 | 38 | 26.0 |
| MNVLO | 118 | 1700 | 4.4 | 390.0 |
| BS1577 | 56 | 2500 | 31 | 81.0 |

The Kd was then determined between hrPrP and a collection of small RNAs, each predicted to contain a substantial amount of secondary structure (FIG. 12). Reactions were incubated for thirty minutes to ensure equilibrium had been reached (data not shown). Surprisingly, each of the RNAs tested demonstrated a considerable affinity for hrPrP (Table 6). AP46 is AP1 minus one G-quartet while AP49 replaces the first G-quartet in AP1 with the sequence GAAA. MDV and MNV, RNAs whose structures have been published previously, are template RNAs for Q-beta replicase (Preuss et. al, 1997). MNVUP and MNVLO are derived from a library that contains 20 random nucleotide inserted into the 5′ loop of MNV. BS1577 is a small RNA containing the 1577 rRNA region from Bacillus subtilis (Ash and Collins, 1992, FEMS Micro. Letters, 94:75-80; the teachings of which are incorporated herein by reference). The tightest binding RNA, MNV:AP1, has a K.sub.d of 3.8M and is a composite RNA containing the AP1 aptamer cloned into the Q-beta replicase template RNA MNV. MNV:AP1 was developed to investigate its utility as an amplifiable RNA with specificity for hrPrP. It is also easier to generate than AP1 due to its larger size. Another RNA that can be amplified by Q-beta replicase and binds tightly to hrPrP is RQ11+12. RQ11+12 was originally designed for use in the detection of the HIV-1 Rev protein after processing into a bipartite probe by the RNA-cleaving phytotoxin sarcin (Zeiler et. al, 2000, Proceedings of SPIE Aerosense 2000, 4036:103-14; the teachings of which are incorporated herein by reference). For these reasons, one of the stem-loop structures in RQ11+12 contains a consensus sequence for the HIV-1 Rev Binding Element (RBE) (Iwai et. al., 1992, NAR 20(24):6465-72; the teachings of which are incorporated herein by reference) adjacent to the consensus cleavage site for the phyotoxins sarcin and ricin (S/R) (FIG. 12d)(Endo and Wool, 1982, J. Biol. Chem. 257(15):9054-60; the teachings of which are incorporated herein by reference). These additional sequences may have something to do with increased binding affinity to hrPrP because RQT157, which is essentially RQ11+12 without the stem-loop containing the RBE and S/R domains, formed the least stable complex with hrPrP, even though it still had an impressive apparent Kd of 56 nM. RQT157 is in turn derived from RQ135, an RNA that is efficiently replicated by Q-beta replicase (Munishkin et. al., 1991, J Mol Bio 221(2):463-72; the teachings of which are incorporated herein by reference).

hrPrP Binding Specificity Can be Differentiated by tRNA:

Because all of the RNAs had apparent dissociation constants with hrPrP measuring below 100 nM, the inventors hypothesized that the binding interaction lacks specificity. To challenge this observation, they assayed the binding affinity of the RNAs in their collection to hrPrP in the presence of purified tRNA, which is sometimes included in RNA binding assays to sequester non-specific binding sites. However, it is known that the interactions between the anticodon of tRNAs can form very stable complexes with complementary RNAs (Grosjean et. al., 1976, J Mol Bio 103:499-519; the teachings of which are incorporated herein by reference). Therefore, it was first necessary to demonstrate that the tRNA was not binding directly to our test RNAs and inhibiting their binding to hrPrP by sequestering the RNAs.

A gel shift assay was used to determine if complexes would form between tRNA and a subset of their RNA collection (FIG. 13). After a thirty minute incubation at 37° C. in Binding Buffer A, MNV:AP1 migrates predominantly as a single band on a non-denaturing gel (lane 1). Addition of 10 ng/μL or 100 ng/μL tRNA to the incubation did not alter the electrophoretic mobility of MNV:AP1 (lanes 2 and 3). When the tRNA concentration was increased to 1 μg/μL, however, the pattern of MNV:AP1 migration changes, with the appearance of a second, slower migrating band that contains at least half of the RNA present (lane 4). This banding pattern suggests the formation of a complex between tRNA and MNV:AP1 when tRNA is present at a concentration of 1 ·mu·g/·mu·L. In contrast, RQ11+12 and MNV did not form a complex at any of the tRNA concentrations (FIGS. 13b and c). Because there were no interactions at the lower concentration of tRNA, the inventors chose to work at a concentration of 10 ng/μL tRNA as an inhibitor of non-specific RNA interactions. This concentration equates to approximately a 400 nM concentration of tRNA, which will be in vast molar excess over any of the RNAs in the ensuing assays, yet low enough to maintain confidence that inhibition of binding will not be due to sequestration of RNAs. When the binding of the various RNA species were reexamined in the presence of tRNA, a greater differential in binding affinity was observed (Table 6, column 2). In the presence of the non-specific inhibitor, the measured K.sub.d values ranged from 12 nM to 2500 nM. The RNAs that were minimally affected by the presence of tRNA and maintained K.sub.d values below 100 nM include AP1, the control RNA, as well as MNV:AP1 and RQ11+12. The rest of the RNAs are more severely affected by tRNA, in some cases, by over two orders of magnitude. Several of these RNAs have dissociation constants (Kd) measuring in the micromolar range. The competitive binding data suggests that the level of specificity in binding of the RNAs to hrPrP is highly variable. Yet there are few clues as to what generates this level of specificity for RNAs with apparently disparate structures like RQ11+12 and MNV:AP1. At the very least, this analysis allows one to rule out length and the overall thermodynamic stability of the RNAs as the factors that dictate stable binding to hrPrP in the presence of a vast excess of non-specific competitor RNAs. The effect of RNA secondary structure on hrPrP affinity is underscored by MNVLO and MNVUP, which differ only by a small stem-loop structure, yet their Kd values vary over sevenfold (FIGS. 12b and c).

An RNA Family that Engenders High Affinity, Specific Binding to hrPrP:

In an attempt to elucidate the requirements in RNA necessary to be bound by hrPrp with high affinity and specificity, the inventors analyzed a subset of four RNAs that are different in their nucleotide composition and predicted architecture. AP1 was chosen because it binds to the N-terminus PrP. MNV was also chosen because its affinity was greatly affected by the presence of tRNA and therefore represents an RNA that binds in a non-discriminatory manner to hrPrP. Therefore, MNV is used as an equivalent to non-specific tRNA in the competition experiments because the tRNA used earlier in this study is a heterogenous population and the effect of each tRNA species in binding to hrPrP remains obscure. In particular, it is known that hrPrP has the ability to interact with tRNA.sup.Lys (Gabus et al., 2001, J Biol Chem June 2001b. 1, 276(22):19301-9; the teachings of which are incorporated herein by reference). MNV:AP1 and RQ 11+12 were chosen because both RNAs are amplifiable by Q-beta replicase, bind specifically to hrPrP with high affinity and were least affected by tRNA.

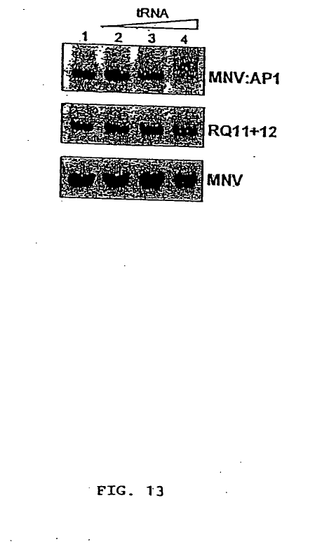

The binding specificity of these RNA species was challenged in competition experiments involving radioactively labeled RNAs and unlabeled competitor RNAs (FIG. 14). The binding of labeled MNV:AP1 (50 pM) to hrPrP (50 μM) was examined in the presence of the competitor RNAs AP1 or MNV over a 5 nM to 120 nM range of concentration. In order to assure that the competitor RNA had a chance to interact with hrPrP, it was always added to the reaction 15 minutes prior to the labeled RNA. After a 30-minute incubation, the extent of binding was measured with the two-filter assay. Under these conditions, AP1 effectively inhibited the binding of MNV:AP1. At a concentration of 120 nM AP1, binding of MNV:AP1 was reduced to below 25%. In contrast, when the competing MNV is present at a concentration of 120 nM, binding of MNV:AP1 to hrPrP is reduced by only 5%. This observation would be consistent with a model where 95% of MNV:AP1 binding to hrPrP occurs via the AP1 sequence. These results also suggest that the AP1 sequence and not the MNV sequence is responsible for specific, high affinity binding to hrPrP. Consistent with this conclusion is the measured Kd's for MNV and AP1 in the presence of tRNA (Table 6).

The binding of RQ11+12 to hrPrP was examined using the same experimental approach. The pattern of inhibition of RQ11+12 binding to hrPrP with the same MNV and AP1 competitors is nearly identical to that of MNV:AP1. Both MNV and AP1 inhibit the binding of RQ11+12 to hrPrP slightly more than they do the binding of MNV:AP1 to hrPrP. This observation is consistent with the slightly lower affinity of RQ11+12 to hrPrP as compared to MNV:AP1. This competition data does not prove, but raises the possibility that RQ11+12 and MNV:AP1 share a common mode of binding to hrPrP.

RNAs Bind to the Amino Terminus of hrPrP:

RQ11+12 was chosen for further characterization because it binds to hrPrP with fairly high affinity and it is resistant to non-specific competition from tRNA or MNV. In addition, RQ11+12 does not interact with tRNA, even at high concentrations (up to 40 μM), unlike MNV:AP1. Finally, RQ11+12 can be rapidly amplified by Q-beta replicase. After testing several buffers, it was determined that the binding of RQ11+12 to hrPrP in the absence of tRNA was improved almost tenfold (Kd=2.2 nM) in 10 mM Tris-OAc, pH 7.5, 100 MM NaCl, 1 mM DTT (Buffer B; data not shown). Binding to hrPrP was also improved for MNV in Buffer B (2.6 nM). Interestingly, in Buffer B, RQ11+12 and MNV have the similar Kd values in the absence of tRNA.

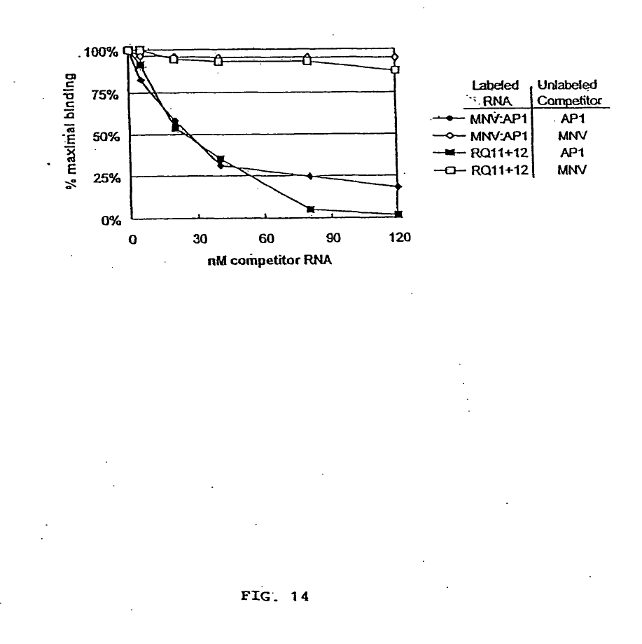

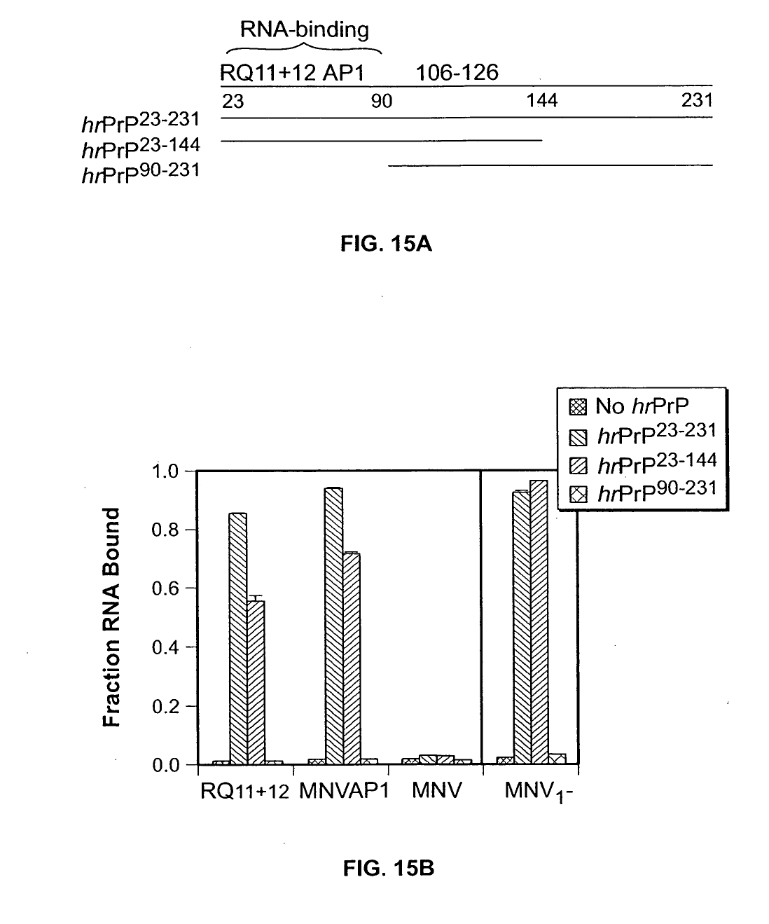

Truncated hrPrP proteins were used to determine the physical location of the RNA binding activity. In addition to the full length recombinant protein, hrPrP²³⁻²³¹, the inventors also examined the N-terminal portion (hrPrP²³⁻¹⁴⁴), and the C-terminal portion (PrP⁹⁰⁻²³¹) using the filter binding assay in Binding Buffer B in the presence of tRNA (FIG. 15). As expected from the results of the competition experiments, both RQ11+12 and MNV:AP1 bind only to full length hrPrP and the N-terminal truncation (FIG. 16b). Because the protein concentration was 20 nM for this set of experiments, MNV was unable to bind to hrPrP in the presence of 10 ng/μL tRNA. However, when tRNA was removed from the binding buffer, MNV binding occurs to either the full length or N-terminal portion of PrP. There was no detectable binding of any of the RNAs to C-terminal truncation, PrP⁹¹⁻²³¹. This data demonstrates that both the discriminatory and non-discriminatory RNA-binding activities of hrPrP reside in the N-terminus of hrPrP, within amino acids 23 to 90.

RQ11+12 and MNV:AP1 Binding is Inhibited by AP1 but not by MNV

To further characterize RNA-hrPrP binding, we used two unlabelled competitor RNAs and examined their effect on the binding of internally labelled RQ11+12 and MNV:AP1 to hrPrP by filter-binding assay. AP1 was chosen as one competitor RNA because it has specific binding activity for hrPrP and its site of binding on the N-terminus of PrP is known. MNV was also chosen as a competitor RNA because it bound non specifically to hrPrP. MNV:AP1 (5 nM) was added to a mixture containing hrPrP (50 nM) and competitor RNAs ranging from 5 to 120 nM. The binding of MNV:AP1 to hrPrP was inhibited by over 80% in the presence of 120 nM AP1. In contrast, MNV:AP1 binding to hrPrP was reduced only 5% in the presence of 120 nM competing MNV. This observation indicates that the high-affinity specific binding of MNV:AP1 to hrPrP is due to the AP1 sequence. The pattern of inhibition by AP1 and

MNV was the same for RQ11+12 binding to hrPrP. At 120 nM AP1, RQ11+12 binding to hrPrP was inhibited by 98%, while inhibition by 120 nM MNV was only 12%. As anticipated, MNV and AP1 inhibit the binding of RQ11+12 to hrPrP slightly more than they do the tighter-binding MNV: AP1. These results imply that RQ11+12 and AP1 bind to the same site.

Protection from RNaseA Degradation Correlates with Binding:

A ribonuclease protection assay was used to examine the nature of the interaction between hrPrP and RNA. If hrPrP interacts minimally with and does not sequester much of the RNA structure, then the RNA should be susceptible to ribonuclease degradation. However, if the RNA/protein complex involves the whole RNA molecule and the interaction with hrPrP sequesters most of the RNA, then the RNA should be stabilized against ribonuclease degradation.

To examine the hrPrP/RNA complexes, RQ11+12 and MNV were incubated with excess hrPrP and allowed to interact for 30 minutes. RQ11+12 was chosen as a representative of discriminatory binding, while MNV was chosen to represent non-discriminatory binding. RNaseA was then added and the incubation continued for an additional 15 minutes (FIG. 16). After this treatment, approximately half of the RNAs remained in samples containing hrPrP, while greater than 95% of the RNA degraded in samples containing RNA alone. If tRNA (10 ng/μ·L) is included in the initial incubation, then the level of protection of MNV imparted by hrPrP is approximately half of that in the absence of tRNA, while the level of protection of RQ11+12 remains unchanged. The pattern of protection of RQ11+12 and MNV directly correlates with their pattern of binding to hrPrP and suggests that complex formation with hrPrP protects bound RNAs.

RQ11+12 Specifically Binds PrP:

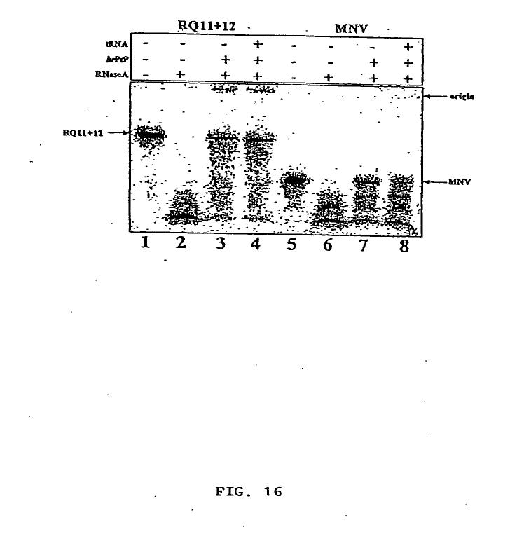

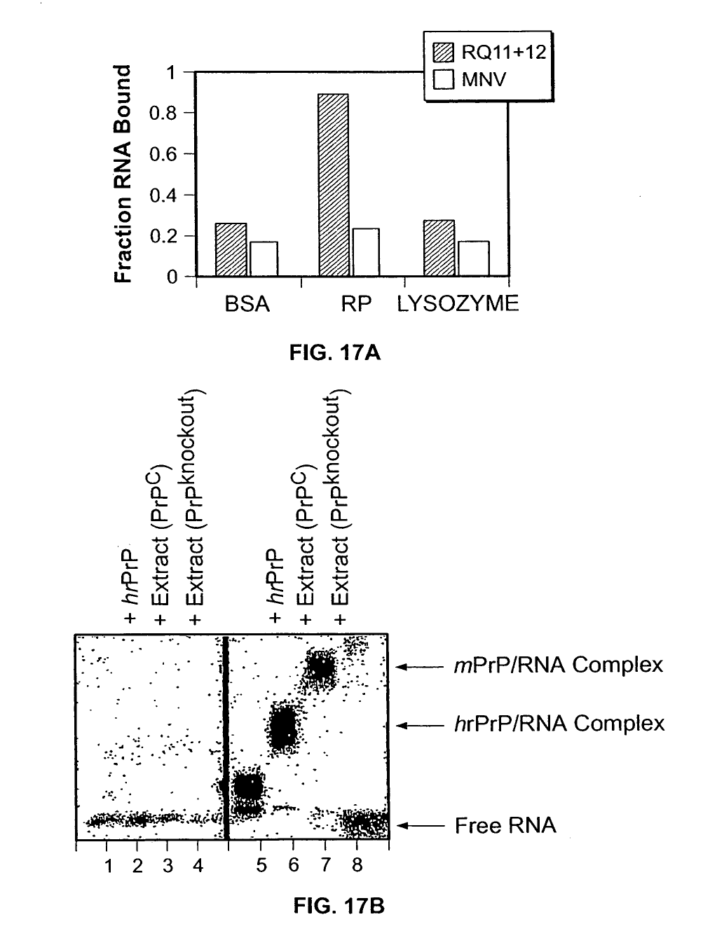

Binding studies in solutions of increasing biological complexity were used to investigate the specificity of RQ11+12 binding to PrP. The theoretical isoelectric point of hrPrP²³⁻²³¹ is 9.39 (ExPASy molecular biology server, Swiss Institute of Bioinformatics) and therefore the protein carries a positive charge in the conditions of the binding assays. As a consequence of its nucleotide composition, the RQ11+12 RNA is negatively charged. Therefore, the positive charge of hrPrP and the negative charge of RQ11+12 raise the possibility that the binding between RQ11+12 and hrPrP is charge-based. To challenge this possibility, the protein lysosyme was used in a binding assay with RQ11+12 because its theoretical p1 is 9.9 (ExPASy) and it should also be positively charged under the conditions of the assay (FIG. 17a). Assay conditions were manipulated (based on the results in Table 6) such that RNA (2 nM and protein (20 nM) concentrations would allow extensive binding for RQ11+12, but not MNV (Binding Buffer A in the presence of 10 ng/·mu·L tRNA). In all cases, MNV binding was at background levels. Binding of RQ11+12 to hrPrP was approximately 90% while binding to BSA and lysozyme was at background levels. These results demonstrate that RQ11+12 does not interact non-specifically with the positively-charged lysosyme protein.

The specificity of RQ11+12 for PrP was further confirmed by examining binding in mouse brain extracts using a gel shift assay. Extracts contain a physiological diversity of proteins, which will provide a stringent test of the specificity of RQ11+12 for PrP. After incubation with 40 ng of hrPrP, migration of labeled RQ11+12 is retarded through a polyacrylamide gel during electrophoresis while MNV migration is affected (FIG. 17b lanes 5 and 6 versus 1 and 2). The specificity of RQ11+12 for PrP is demonstrated after incubation with an equivalent amount (20 μg) of either wildtype mouse brain extract or a mouse PrP-knockout brain extract (lanes 7 and 8). A shift is observed only with the wildtype mouse, implying that the shift observed in lane 7 is due solely to the presence of endogenous PrP present in the wildtype extract. The greater retardation of RQ11+12 in lane 7 than in lane 6 is possibly due to either the post-translational modifications present in native PrP and lacking in hrPrP, the association of PrP.sup.C with host proteins or the presence of PrP dimers that form in vivo and not in purified, recombinant PrP (Yeheily et al., 1997, Neurobiology of Disease 3:339-355; Meyer et. al., 2000, J Biol Chem Dec. 1, 275(48):38081-7; the teachings of which are incorporated herein by reference). The pattern of interaction between the RNAs and native mouse PrP in extracts is reminiscent of the filter binding experiments done in the presence of tRNA where RQ11+12 shows high level specificity and MNV does not.

RQ11+12 Promotes Fibrillation of hrPrP:

In recently published studies, it was reported that DNA can induce the fibrillation of recombinant PrP in vitro. In these experiments, a 1500 nt double stranded DNA, comprised entirely of G-C base pairs (gcDNA), was incubated at low pH (5.0) with purified, recombinant protein. Using the aggregation specific dye Congo Red, the inventors were able to demonstrate DNA-dependent fibrillation. In addition, this PrP/DNA aggregate was resistant to digestion by proteinase K, a hallmark of PrP^{C} structural transformation.

The inventors were interested if RQ11+12 had the same ability. Unlike the gcDNA used in Nandi's studies, RQ11+12 does not self-aggregate and therefore they chose to demonstrate the results of hrPrP/RQ11+12 complex formation by electron microscopy (FIG. 18). Rather than using the acidic conditions of Nandi, they chose a buffer with a physiological pH (50 mM MOPS pH 7.5, 0.1 μg/U.S. Pat. No. L tRNA, 8 μg/μL bovine calf serum, 0.05% NP-40/deoxycholic acid). After a 16 hour incubation at room temperature, 8.9 pmoles of hrPrP did not form a discernible superstructure (FIG. 18a). However, if 0.16 pmoles of RQ11+12 were present in the reaction, an aggregated structure is can be observed (FIG. 18b). Although the superstructure formed between hrPrP appears to form a lattice-like aggregate, as opposed to SAFs recovered from scrapie-infected tissue that form discrete rod-like structures, the effect of RQ11+12 on hrPrP aggregation is clear and apparent. This study confirms and extends the previously reported observations that ribonucleic acids bind to recombinant PrP. The results presented here demonstrate that hrPrP can form very stable complexes with several different RNAs with dissociation constants in the low nanomolar range. Two RNAs, RQ11+12 and MNV:AP1, bound to hrPrP very tightly and showed a resistance to competition from other RNAs and thereby showed a marked difference in binding activity as compared to some of the other RNAs in their test group. In addition, RQ11+12 presumably bound specifically to endogenous PrP^{C} in whole mouse brain extracts. Although there are few apparent similarities between MNV:AP1 and RQ11+12 that would explain their specific binding to hrPrP, both are predicted to have secondary structural elements that contain thermodynamically stable non-canonical base pairs, a feature common to protein-binding RNAs. Non-Watson-Crick base pairs can widen the RNA deep groove, thereby accommodating interactions with extended protein domains and exposing additional hydrogen bonding groups that may play a role in protein ligand-binding specificity (Ye et al., 1999 Nat Struct Biol. 3(12):1026-33; Jiang et al., 1999 Structure Fold Des. 7(12):1461-72; Hermann and Westhof, 1999 Chem Biol 6(12)-R33543; Patel, 1999 Curr Opin Struct Biol 9(1):74-87; Puglisi and Williamson, 1999 in the RNA World, R F Gesteland, T R Cech, J F Atkins, eds., Cold Spring Harbor Laboratory Press, Cold Spring Harbor, N.Y., 2d, 1999, pp 403-25; Hermann and Patel, 2000 Science 287(5454):820-5; the teachings of which are incorporated herein by reference). The previously described AP1 structure in MNV:AP1 has three stacked G-quartets, while RQ11+12 contains two RNA domains known to contain non-Watson-Crick base pairs—the Rev Binding Element (RBE) and the Sarcin/Ricin cleavage domain from 18S rRNA (FIG. 12d)(Iwai et al., 1992; Endo and Wool, 1982 J Biol Chem 257(15):9054-60; the teachings of which are incorporated herein by reference). The thermodynamic stability imparted by these RNA secondary structures may make possible a more extensive interaction with the flexible N-terminus of hrPrP. The unstructured peptide may alter its conformation to the architecture of the stable RNAs, thus forming a stable complex, as is the case in the binding between peptides derived from the HIV-1 Rev protein and its RNA aptamers (Ye et al., 1996; Ye et al., 1999 Chem Biol 6(9):657-69; Tan et al., 1993 Cell 73(5):103140; the teachings of which are incorporated herein by reference).

It was proposed that DNA can lower the energy of activation in the conversion of PrP^{C} into PrP^{Sc} by stabilizing a PrP intermediate through the formation of a DNA/protein complex. In this study, the inventors present data that may suggest a similar role for RNAs like RQ11+12. They demonstrate that RQ11+12 binding to PrP occurs at very low concentrations with high affinity and specificity in the presence of a vast molar excess of competitor RNA. The binding of hrPrP also protected RQ11+12 from degradation by RNaseA. These results present a model that a specific RNA interaction, typified by the interaction of RQ11+12 with hrPrP, could initiate the formation of RNA/PrP complexes, and even catalyze the formation of PrP aggregates. Such aggregates are predicted to be fibrillar and insoluble. Formation of such an aggregate may explain the amount of sample accumulated at the origin in the denaturing gel in FIG. 16, lanes 3 and 4. This may also explain the results of the self-competition binding experiments where RQ11+12 competes for its own binding to hrPrP more efficiently than MNV competes for its own binding (FIG. 16b). The effect becomes most apparent when the ratio of PrP to RNA approaches 1:1, which would be expected if RQ11+12 was binding to more than one molecule of hrPrP. Previously, the in vitro formation of PrP aggregates catalyzed by nucleic acids has been demonstrated with DNA under acidic conditions (Nandi and Leclerc, 1999; Nandi and Sizaret, 2001; Cordeiro et. al., 2001 J Biol Chem 276(52):49400-09; the teachings of which are incorporated herein by reference). It is tempting to suggest that RQ11+12 catalyzes the formation of RNA/PrP aggregates. The effect is kinetic RNAs like RQ11+12 can interact with PrP in a very particular way that can efficiently catalyze structural transformation. Due to the specificity of the interaction, it can occur at low concentrations. This activity may be related to the difference in binding activity between RNAs like RQ11+12 and RNAs like MNV.

Due to the complexities of prion disease transmission, some recent studies have focused on the possibility that a cofactor may exist that assists in the initiation or progression of prion disease (Weissmann, 1991 Nature 352:679-83; the teachings of which are incorporated herein by reference). The search for a “factor X” protein that catalyzes the transformation from PrP^{C} to PrP^{Sc}, the hallmark of prion diseases, has uncovered several possible protein candidates. Recently, it has been demonstrated that DNA can bind to PrP in vitro and induce fibrillation under special conditions (Nandi, 1998; Nandi and Leclerc, 1999; Nandi and Sizaret, 2001; Cordeiro et. al., 2001). This observation led one group to propose that DNA might play a role in the progression of prion diseases. The present data suggests that RNAs like RQ11+12 may be a more likely candidate for involvement prion disease. The K.sub.d of RQ11+12 for hrPrP is three orders of magnitude less than that measured for dsDNA, binding occurs under physiological conditions and the RNA would have the opportunity to interact with PrP^{Ntm}, whose N-terminal domain extends into the endoplasmic reticulum (Hedge et al., 1998 Science 279(5352):827-34; the teachings of which are incorporated herein by reference). A model for the induction of PrP aggregation can be built around the structural change that the N-terminus of PrP undergoes upon the binding of a specific RNA ligand. The new conformation of the N-terminus may activate the fibrillation activity of the conserved amyloidogenic region of PrP (amino acids 106 to 126) by exposing it to the solvent. A similar mechanism is followed: in the fibrillation of the 90 kDa amyloid precursor protein (APP) which aggregates only after the internal 42 amino acid β-amyloid peptide is excised from APP and becomes solvent exposed (Rochet and Lansbury, 2000 Curr Opin Struct Biol 10:60-8; the teachings of which are incorporated herein by reference). If this model were correct, the present results would suggest that an RNA molecule might be capable of inducing or accelerating fibril formation in prion diseases.

Regardless of the mechanism of binding to PrP or the role of specific RNAs in prion diseases, the inventors demonstrate that the RNA RQ11+12 is a capable probe for the PrP protein In addition, RQ11+12 is a potent template for the RNA-dependent RNA polymerase, Q-beta replicase. This new type of bifunctional RNA molecule or amplibody has the ability to specifically bind to a protein target and to be amplified directly by Q-beta replicase. Molecules like RQ11+12 are useful not only in the study of prion biology, but also in the potential development of newer, more sensitive diagnostic technologies.

Small, Highly Structured RNAs (shsRNAs) Bind Human Recombinant Prion Protein (hrPrP) with High Affinity and Specificity

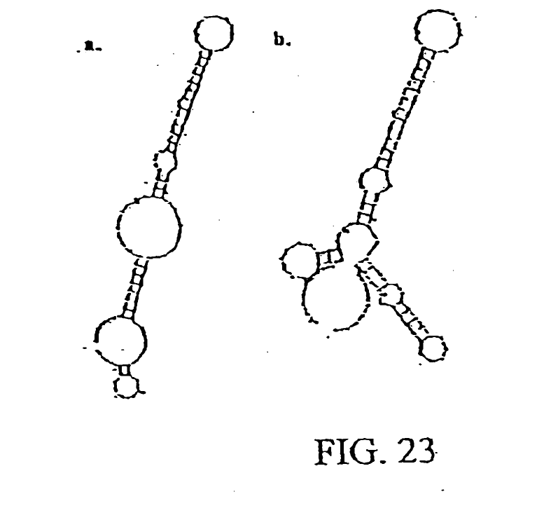

The binding activity of human recombinant PrP (hrPrP) for a number of shsRNAs was characterized by gel-shift assay. The RNAs were chosen on the basis of their ability to bind to hrPrP by filter-binding assay in buffer at physiological pH 7.5 (96). The RNAs SC-2, and a variant containing a point mutation, SC-4, were added to this study because they were related to aptamers reported by Stefan Weiss (CEA) to specifically bind to PrPSC (reported at the PittCon 2001 Symposium, New Orleans, La, Mar. 4-9, 2001). Similar to the other RNAs used in this study, SC-2 and SC-4 are relatively short and contain significant secondary structure as predicted by computer-assisted structural analysis (FIGS. 23a and b) (97).

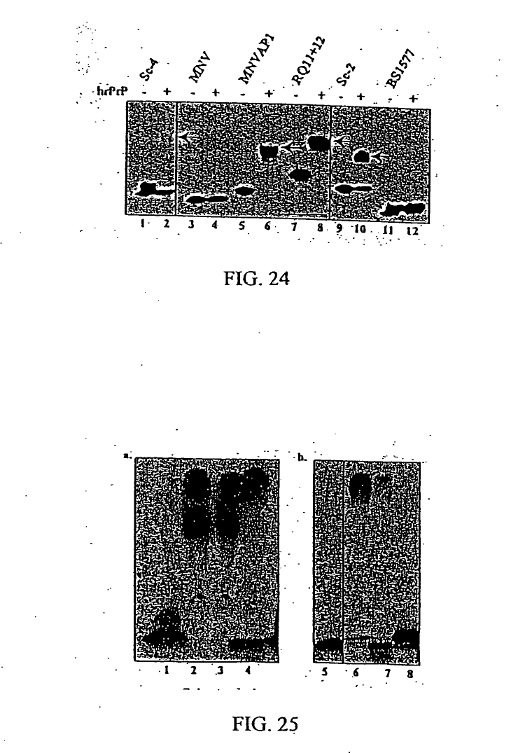

The hrPrP protein carries a positive charge at physiological pH (pI=9.39) and therefore it was necessary to use semi-denaturing PAGE conditions (gels contain urea and are run at room temperature) in order for ribonucleoprotein complexes to enter the gel to visualize the extent of complex formation (see Materials and Methods). In an effort to approximate physiological conditions in vitro, binding reactions were done at pH 7.5, in the presence of serum components (10% BCS; ˜80-100 mg/ml) and tRNA (1 mg) (FIG. 24). Under these conditions, the migration of internally labeled RNAs (1.75 nM) was proportional to the length of the transcripts, indicating that the shsRNAs examined did not form stable complexes with components present in BCS (FIG. 24, lanes 1, 3, 5, 7, and 9). However, the presence of hrPrP (55 nM) during incubation resulted in the retarded migration of RQ11+12, presumably due to specific nucleoprotein complex formation (lane 8). Similar results were observed with MNVAP1 RNA (lane 6), consistent with an earlier report that RQ11+12 and MNVAP1 form stable complexes in vitro with hrPrP in the presence of excess tRNA (96). The RNAs MNV and BS 1577, as expected from the previous study, did not form complexes with hrPrP under the conditions of this assay. SC-2 and SC-4 demonstrated intermediate affinity for hrPrP; the majority of the signal relating to SC-2 was shifted in complex with hrPrP (lane 10), while only a small part of the signal relating to SC-4 shifted (lane 2). These results indicate that these four shsRNAs are capable of forming stable complexes with hrPrP in the presence of serum proteins. Based on the fraction of band shifts, RQ11+12 and MNV-AP1 had highest-binding activity to hrPrP, while Sc-2 showed intermediate binding activity and Sc-4 showed low-binding activity. A probable explanation for the variation in intensity of the radioactive bands representing the nucleoprotein complexes is that the RNAs vary in affinity to hrPrP under these conditions. In addition, because nucleoprotein complex formation occurred only in the presence of hrPrP (in a background of BCS proteins), these RNAs demonstrated specificity for hrPrP.

The monoclonal antibody (mAb) 7D9, directed against recombinant mouse PrP, was used to confirm that PrPC is present in the ribonucleoprotein complexes (FIG. 25a). If RQ11+12 binding to PrP induces a gel shift, then the addition of a monoclonal antibody specific to PrP should induce a supershift of the complex if the binding of the antibody to PrP does not compete with RQ11+12. Internally labeled RQ11+12 RNA was incubated in 10% BCS and separated by semi-denaturing electrophoresis, as above. In the absence of PrP, RQ11+12 runs towards the bottom of the gel (lane 1). The mobility of RQ11+12 is unchanged by the addition of 7D9. In the presence of mouse brain extract (MBE), the signal representing RQ11+12 is significantly retarded, indicating an interaction between RQ11+12 and a component in the MBE (lane 2). This signal is not changed when a mAb to an unrelated protein is added to the reaction mix (lane 3). However, the addition of PrP-specific mAb 7D9 to the reaction mix results in a supershift of the RQ11+12 signal (lane 4), confirming that the shifts in RQ11+12 migration is due to complexes containing endogenous mouse PrP.

A gel shift experiment was used to further demonstrate that the component in the MBE extract that interacts with RQ11+12 is PrP (FIG. 25b). The electrophoretic migration of internally labeled RQ11+12 incubated in the presence of MBE is severely retarded (compare lanes 5 and 6). A similar migration was observed between hrPrP and RQ11+12 (lane 7). However, if the MBE was prepared from a PrP knockout mouse (PrP0/0), there is no shift in RQ11+12 migration (compare lanes 8 and 5), demonstrating the requirement of PrP for complex formation.

shsRNA Interacts with PrPC in Brain Homogenates From Mouse Rat and Hamster

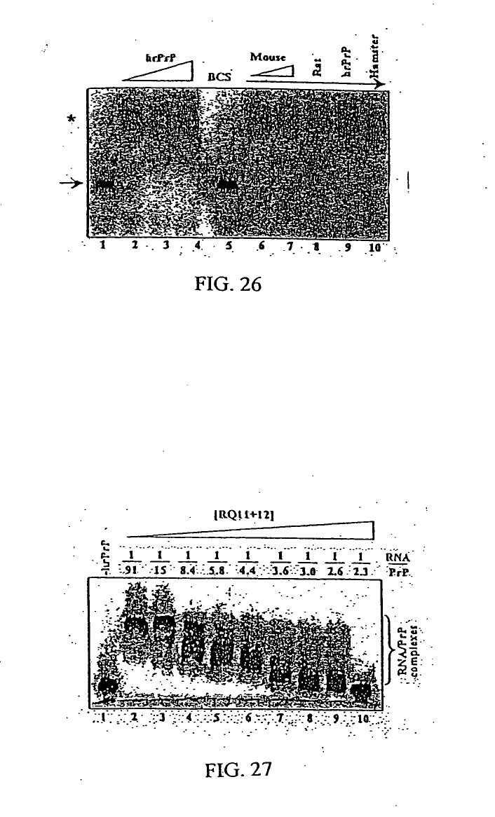

Additional gel-retardation assays were carried out using brain homogenates from mouse, rat and hamster to determine if RQ11+12 can interact with endogenous PrP^{C} from organisms other than mouse (FIG. 26). During electrophoresis, internally labeled RQ11+12 migrated as a single band whether incubated in TE buffer (lanes 1-4) or in the presence of 10% BCS (lanes 5-10), once again, indicating that the RNA does not form stable complexes with bovine serum proteins (compare lanes 1 and 5). The addition of increasing amounts of hrPrP to the reaction mix caused a shift in the band representing RQ11+12 (lanes 2-4). The shifted bands represent stable nucleoprotein complexes that are resistant to denaturation during semi-denaturing electrophoresis in the presence of urea and SDS, as seen in FIG. 24, lane 8. Bands of similar intensity and migration are also apparent in lanes containing brain extracts prepared from mouse (FIG. 26, lanes 7 and 8), rat (lane 8) and hamster (lane 10). These results demonstrate RQ11+12 is not species-specific and has the ability to bind to recombinant human PrP and PrPC from different origins with high specificity.

The Apparent Mass of Nucleoprotein Complexes is Affected by the RNA:hrPrP Ratio

The migration of RQ11+12/hrPrP complexes in semi-denaturing electrophoresis conditions appears to be subject to the ratio of PrP to RNA. Increasing the ratio of PrP to RNA appeared to slightly retard the migration of the nucleoprotein complexes whether it was a recombinant form of the protein (FIG. 26, lanes 24) or endogenous mouse PrP^{C} (lanes 6 and 7). To further examine the effects of PrP:RQ11+12 ratios on the migration of stable complexes, a constant amount of hrPrP was incubated with varying amounts of RQ11+12 and complex formation was visualized by gel shift. In order to maintain a constant radioactive signal while increasing RNA concentration, unlabeled RQ11+12 was added to a constant amount of internally labeled RQ11+12. Under these conditions, retardation of the migration of the radioactive signal is presumably due to increased mass of nucleoprotein complexes.

The apparent mass of the nucleoprotein complexes varied depending on the RNA:hrPrP ratio in the reaction mix where these complexes were formed (FIG. 27). In the absence of hrPrP, RQ11+12 has the highest electrophoretic mobility and runs towards the bottom of the gel (lane 1). At the lowest concentration of RNA, where the highest RNA:protein ratio was achieved (1:91), the RQ11+12/hrPrP complex runs with the lowest mobility as a relatively tight, discrete band (lane 2). As the concentration of RNA is increased, thereby decreasing the ratio of RNA to protein, the migration of the complexes increase and they run as smeared bands provided that the ratio is above 1:3 (lanes 3-7). When the RNA:protein ratio falls below a 1:3 ratio, the bands representing the complex become tighter and shift less, until, at a ratio of 1:2.3, there is no apparent shift at all (lane 10). These results, and the results presented in FIG. 26, suggest that at high PrP:RNA ratios, several PrPC molecules bind to a single RQ11+12 molecule. At a ratio of 1:15, the maximum observed shift occurs (lane 3). At a higher ratio, the signal is less smeared but the amount of maximal shift did not increase, suggesting that RQ11+12 is saturated with hrPrP when the ratio is 1:91 (compare lanes 2 and 3). The absence of a noticeable shift at lower RNA:protein ratios reflects either the formation of nucleoprotein complexes with no significant increase in mass or that complexes formed between one RNA and one hrPrP were weak and could not withstand the conditions of electrophoresis.

Nucleoprotein Complex-Associated PrP is Partially Resistant to Proteinase K Degradation

It has previously been reported that endogenous PrP^{C} purified from extracts can be converted into isoforms that are insoluble and resistant to PK degradation (PrPRes) by either seeding the reaction with PrP^{Sc} or adding 200 mM copper. Several observations lead us to think that nucleoprotein complexes consisting of high hrPrP:RQ11+12 ratios might also be resistant to PK digestion. Firstly, nucleoprotein complexes formed between RQ11+12 and hrPrP are extraordinarily stable and do not dissociate during electrophoresis in the presence of 7 M urea and detergents (see Materials and Methods; gel-retardation assay). Secondly, nucleoprotein complexes lost their solubility and could be recovered by centrifugation for five minutes at 10,000 g. Finally, preliminary electron microscopy studies demonstrate that the addition of RQ11+12 RNA induces hrPrP to form amyloid-like aggregates.

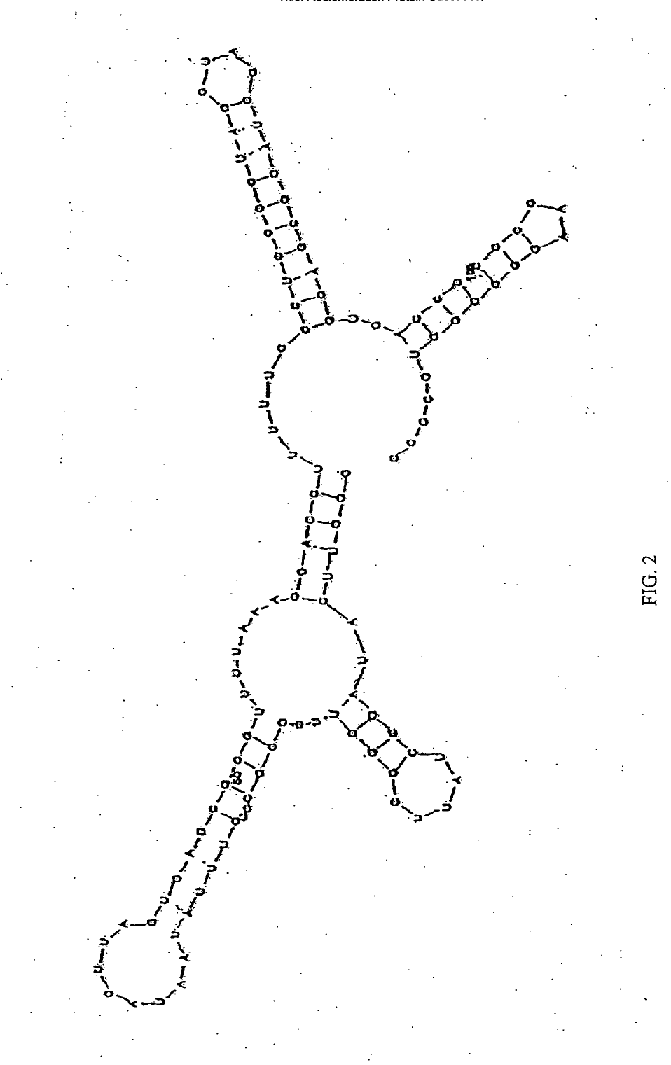

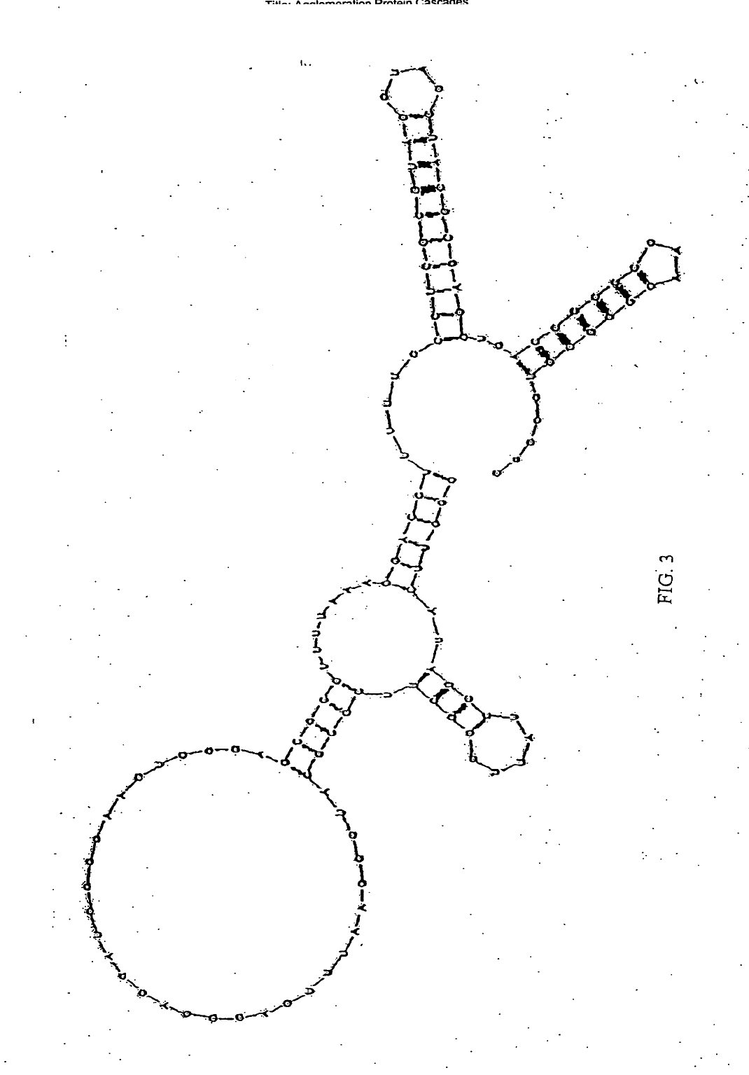

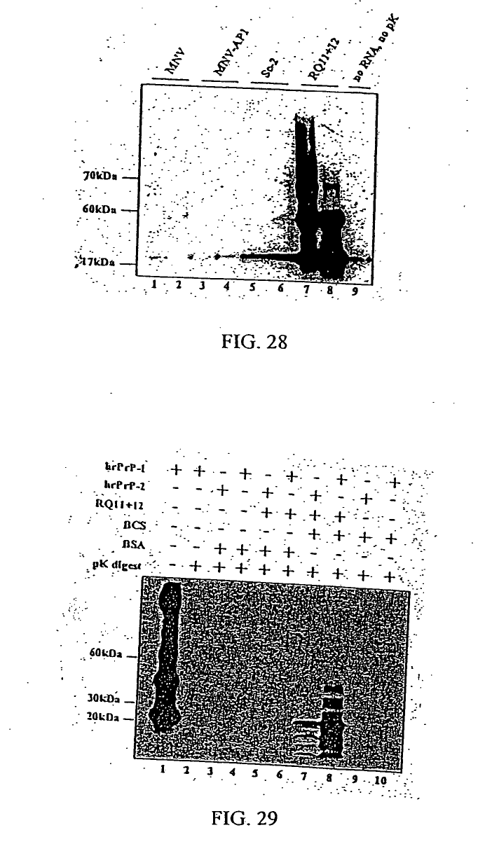

A PK-protection assay was used to characterize the ability of RNAs to convert hrPrP into PrPRes (FIG. 28). hrPrP (83 pmol) was incubated with two different concentrations of RNA (0.2 pmol and 1.2 pmol), digested with PK (100 mg/ml), and the remaining PrP (PrPRes) was visualized by Western analysis. Under these conditions, RQ11+12 effectively induced hrPrP to become resistant to PK proteolysis (FIG. 28, lanes 7 and 8). The effect appears even greater when the RNA is at a lower concentration. In contrast, MNV did not induce hrPrP resistance to PK (lanes 1 and 2), consistent with its inability to bind PrP under similar conditions (FIG. 2, lane 4). The SC-2 RNA generated a low level of protection from PK digestion relative to RQ11+12 (FIG. 28, lanes 5 and 6), which also correlates with its lower-binding affinity (FIG. 24, lane 10). Curiously, MNV-AP1, which demonstrated high affinity for hrPrP in the gel-shift assay (FIG. 24, lane 6), provided almost no protection against PK digestion (FIG. 28, lanes 3 and 4). These results suggest that although binding to hrPrP is prerequisite for the generation of PrPRes, it is not the only criterion that dictates protection from PK digestion, suggesting that complex interactions between RQ11+12 and PrP occur following initial binding. Significantly, this is the first description ever of the generation of PrPRes from recombinant PrP and RNA at physiological pH in the absence of PrP^{Sc}.

The PK-protection assay was again used to further characterize the components required for the in vitro conversion of PrPSen to PrPRes in the presence of RQ11+12. Test mixtures containing PrPSen and various components were incubated for 30 minutes to allow ribonucleoprotein complexes to form, then digested with 50 mg/ml PK, and examined by Western blots to visualize PrPRes (FIG. 29). In this experiment, PrPSen was provided by two separate preparations of hrPrP (hrPrP-1, hrPrP-2). The PrP from each preparation was completely digested by PK (lanes 2 and 3), even in the presence of 100 mg BSA (lane 4). The addition of RQ11+12 did not induce PrPRes formation in the presence of BSA (lanes 5 and 6). Only when both RQ11+12 and BCS were present was a significant amount of PrP detectable (lanes 7 and 8), suggesting formation of PrPRes. The difference in detectable PrPRes correlates with the difference in the initial amount of PrP in hrPrP-1 and hrPrP-2 samples. If RQ11+12 was removed from this mixture, and only BCS was present, the conversion to PrPRes is barely detectable (lanes 9 and 10). Taken together, these data demonstrate that a component present in BCS is necessary for the in vitro conversion of PrPSen to PrPRes under these conditions. Variability in sustaining PrPSen→PrPRes transformation was observed between BCS preparations from different vendors and different lots from the same vendors. Significantly, these results suggest that an unknown component(s) present in BCS, together with RQ11+12, participates in PrPSen→PrPRes transformation.

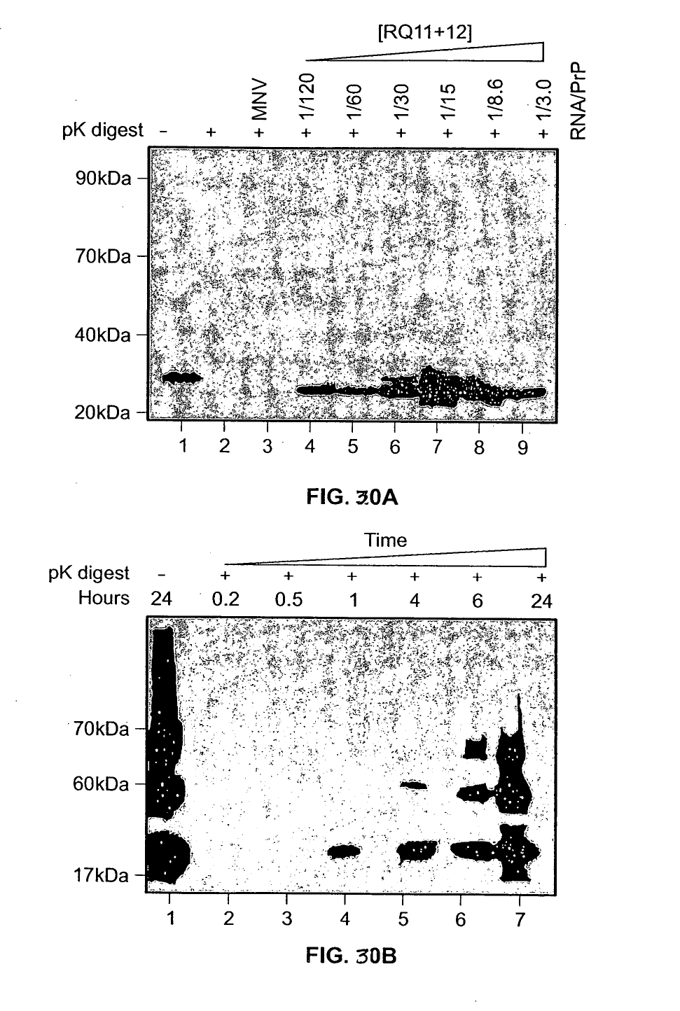

The ratios of RQ11+12 and hrPrP were varied and subjected to the same PK digestion assay to further characterize the ability of RQ11+12 to convert hrPrP to PrPRes in vitro at physiological pH (FIG. 30a). The ratios were achieved by incubating a constant amount of hrPrP (1.2 pmol) with increasing amounts of RQ11+12 RNA (0.01-0.4 pmol). The PK treatment was sufficient to digest all 1.2 pmol of hrPrP to below levels of detection (FIG. 30a, lane 2). MNV RNA (0.1 pmol) was unable to induce the formation of PrPRes (lane 3). In contrast, all of the concentrations of RQ11+12 were able to induce the formation of PrPRes (lanes 4-9). The effect appears to be dependent on the ratio of the two components, with the most PrPRes generated at a RNA:protein ratio of 1:15 (lane 7). Interestingly, adjusting the ratio so that it approached 1:1 by increasing the amount of RNA appeared to diminish the protective effect against PK. These results are in agreement with the results of the gel retardation experiments, where the maximum mass of nucleoprotein complex is first achieved when the RQ11+12/hrPrP molar ratio is 1:15 (FIG. 27, lane 3).

The formation of the PK resistant nucleoprotein complex in the presence of serum was timedependent (FIG. 30b). 200 ng of hrPrP (8.7 pmol) were incubated with 38 ng of RQ11+12 RNA (0.6 pmol) in binding buffer at 37° C., as above. At various time points, the incubation mixtures were treated with PK. After one hour, residual PrP can be detected by Western analysis with mAb 3F4. The amount of remaining PrPRes increases as a function of time. As the amount of PrP increases, three major hrPrP bands are routinely observed, presumably relating to monomer, dimer and trimer forms of the protein. After 24 hours, we estimate that approximately 60% of the initial hrPrP has been converted into a PK resistant form. These results demonstrate that the conversion of PrPSen to PrPRes in our system is very rapid and requires no special manipulations.

RNA in Nucleoprotein Complex is Resistant to RNase A

A number of observations lead us to investigate if the RNA in complex with hrPrP was resistant to ribonuclease digestion. The flexible N-terminal region of PrP, implicated in nucleic acid binding, has the capacity to wrap around a bound ligand, thereby shielding it from the solvent. EM studies have demonstrated that the conformation of linear DNA is altered upon PrP binding forming a condensed, globular aggregate structure. In addition, our own observations suggest that up to 15 molecules of PrP bind to one molecule of RQ1+12, enough to potentially block access to the RNA by nucleases in solution.

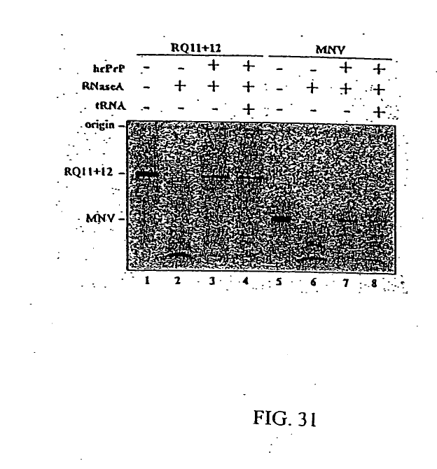

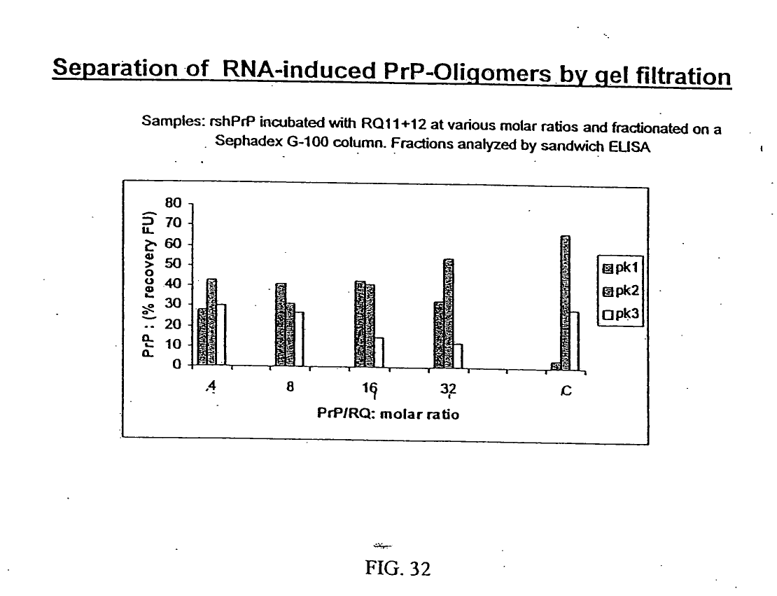

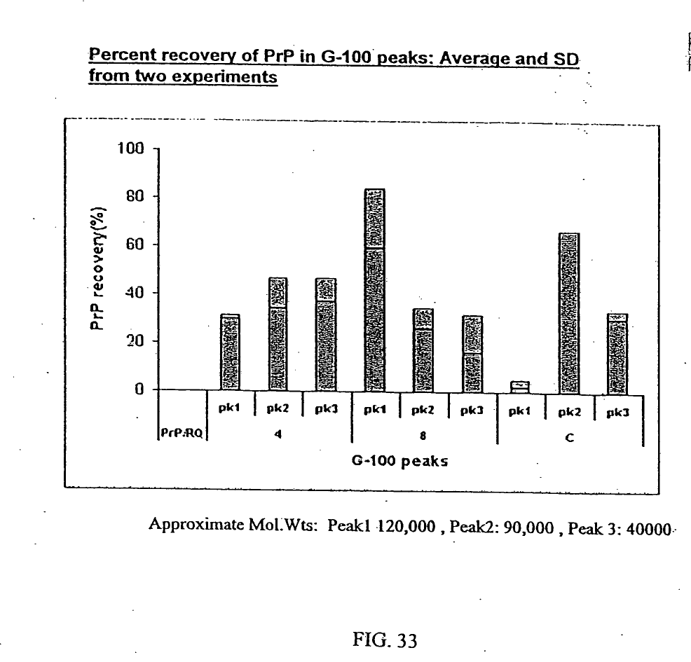

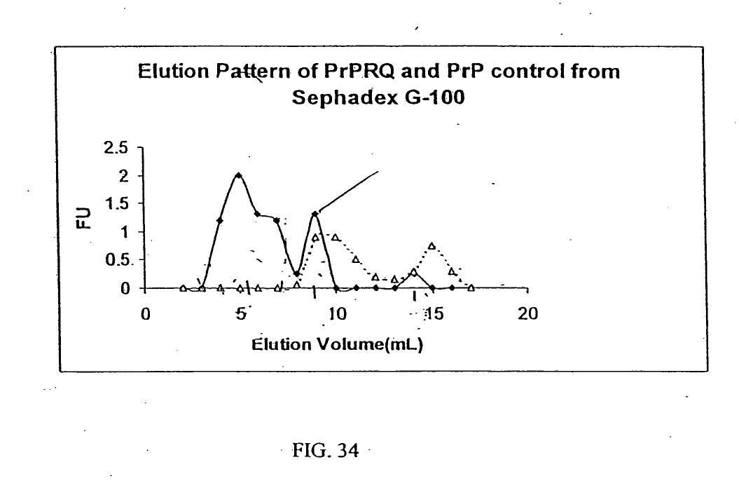

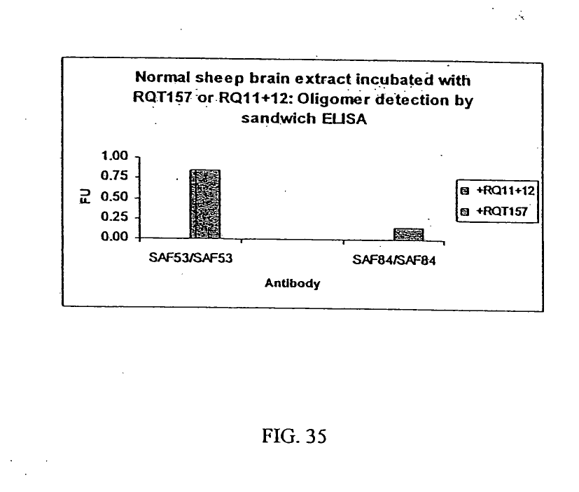

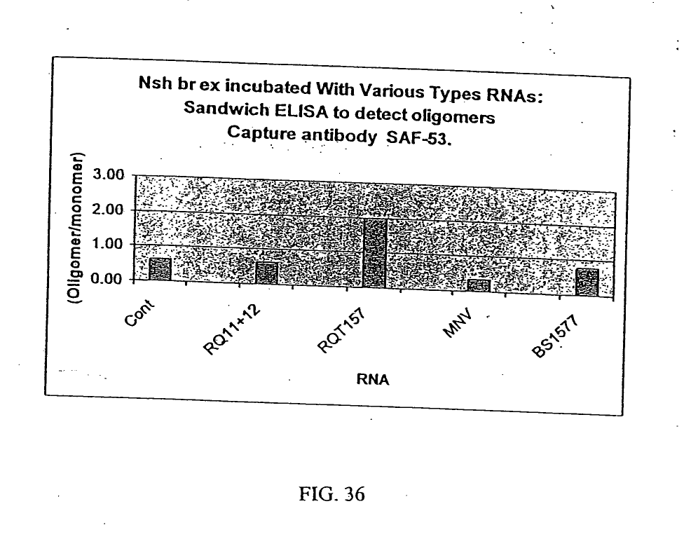

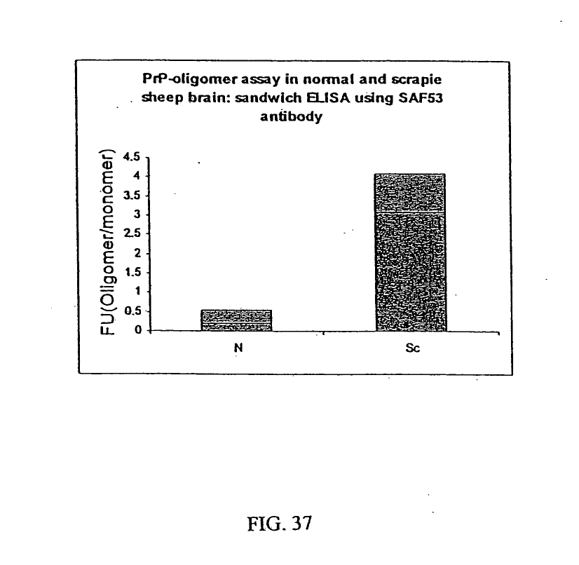

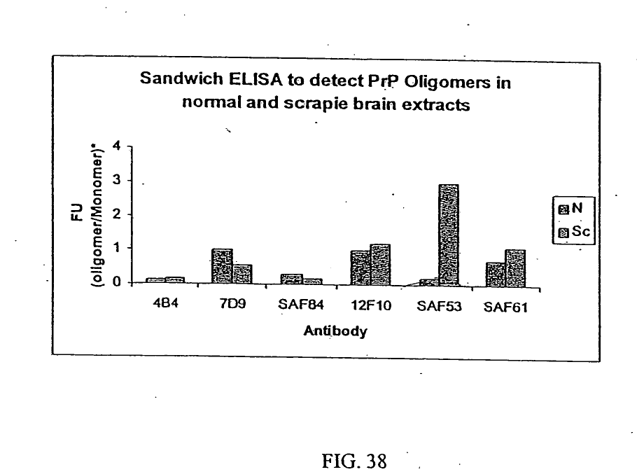

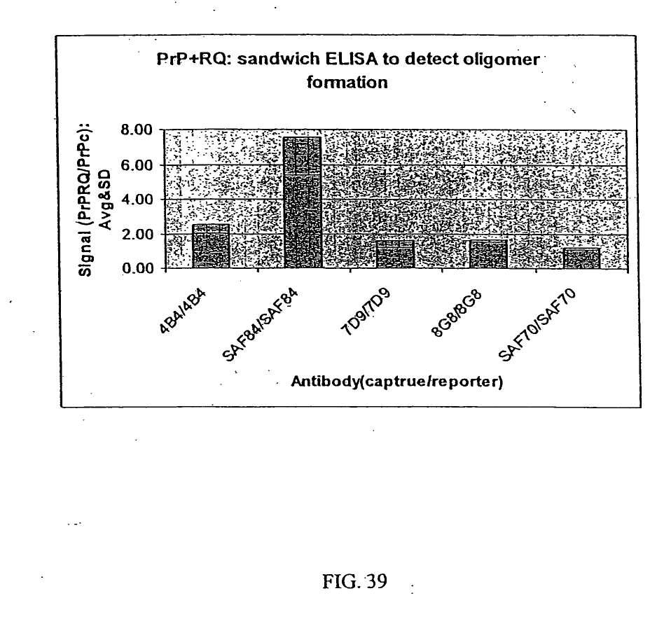

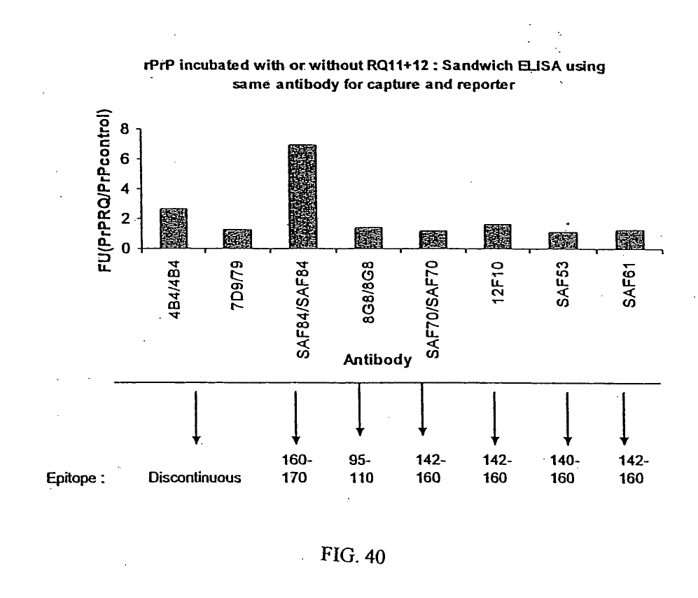

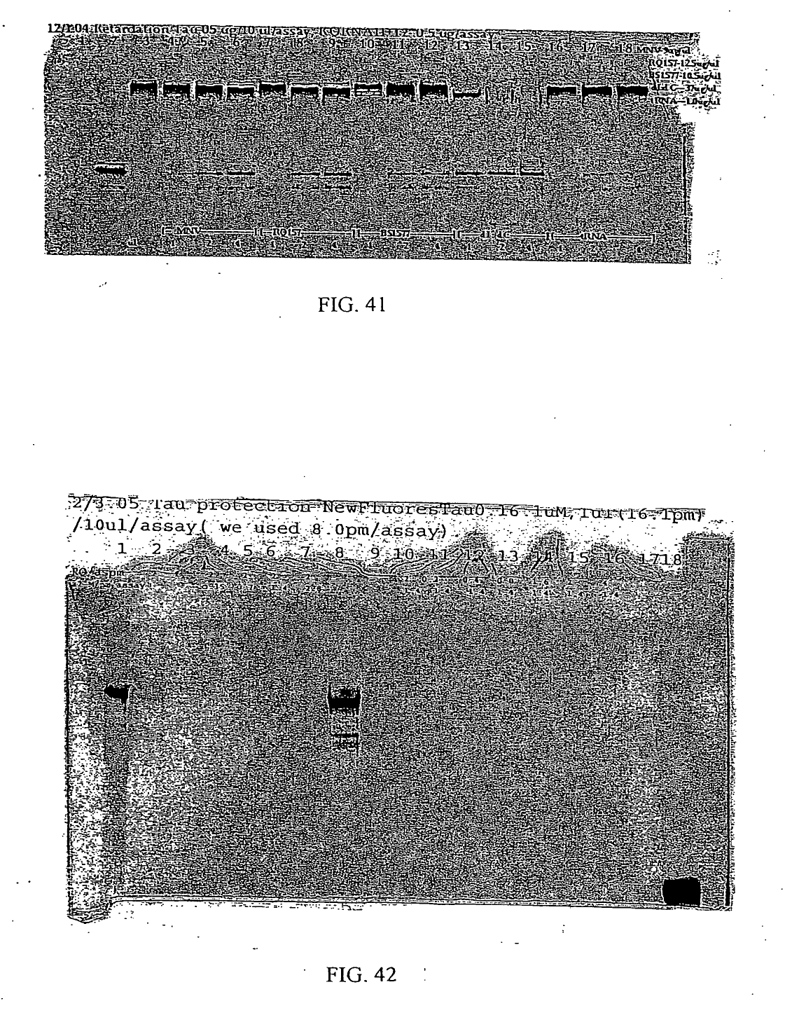

Nucleoprotein complexes were subjected to digestion by ribonuclease A (RNase A) to examine if RNA in complex with hrPrP was resistant to ribonuclease digestions. Internally labeled RNAs and hrPrP were incubated in a buffer lacking serum and tRNA (see Materials and Methods) prior to the addition of RNase A (FIG. 31). In the absence of hrPrP, RQ1+12 was degraded into small fragments with less than 2% full-length material remaining after RNase A digestion, as determined by phosphorimage analysis (compare lanes 1 and 2). However, if hrPrP was present prior to the addition of ribonuclease, greater than 50% of full-length RQ11+12 remained intact (lane 3). A similar effect was observed for MNV RNA (lanes 4-6), consistent with results obtained in an earlier study demonstrating that hrPrP has a high affinity for MNV in the absence of tRNA. The same study demonstrated that the presence of tRNA significantly decreases the affinity between MNV and hrPrP, but does not have the same effect on RQ11+12/hrPrP binding. As expected, the addition of tRNA during nucleoprotein complex formation decreased the amount MNV remaining after nuclease digestion to approximately 25% (lane 8). The addition of tRNA to the reaction mixture did not alter the protective effect of hrPrP on RQ11+12 against RNase A (lane 4). These results demonstrate that hrPrP binding directly correlates with protection of RNAs from ribonuclease degradation.

Materials and Methods

RNA preparation

The shsRNAs were synthesized by in vitro transcription with T7 RNA polymerase (Ambion, Epicentre) from DNA templates derived from sequenced plasmids. The RNAs were purified by organic extraction and precipitation, and then further purified by excision from denaturing polyacrylamide gels.

All of the RNAs used in this study (Table 7) have previously been shown to bind to hrPrP in simple buffer in the absence of BCS components. The origins and predicted secondary structures for MNV, MNVAP1, RQ11+12 and BS1577 have been described elsewhere.

Proteins and Monoclonal Antibodies (MAbs)

Monoclonal antibodies (mAbs) were acquired from commercial sources. The mAbs for PrP protein 3F4 (31), directed against amino acid residues 109-112 of hamster and human PrP, and 7D9, directed against a non-linear epitope in amino acid residues 23-237 of recombinant PrP, were purchased from Signet Laboratories. The monoclonal antibody against JNK was from New England Biolabs/Cell Signaling Technology.

A 646 bp DNA fragment-containing nucleotides coding for amino acid residues 23-231 of human PrPC followed by TAG and TAA stop codons were amplified by PCR from commercially available cDNA of human brain (ATCC, clone pmPrP3, number 65847, Acc.:X02514). An Nde I restriction site, containing the initiating ATG codon, was introduced into the 50-primer: (50-TATCATATGGAAGAAGCGCCCGAAGCCTGGA-30) and a Xho I restriction site and stop codons were introduced into the 30-primer: (50-GAACTCGAGTTACTAGC TCGATTCTCTCTGGTAATAGGC-30). The amplified products and the pET21 d( [symbol] ) vector (Novagen) were digested with Nco I and Xho I, and then ligated using phage T4 DNA ligase to create a recombinant expression plasmid (pEHPrP23-231). The structure of cloned fragment was confirmed by DNA sequencing.

A saturated overnight culture of E. coli BL21 (DE3) pLys S containing pEHPrP23-231 was diluted 1:20 with 2YT medium supplemented with 100 mg of ampicillin and 34 mg of chloramphenicol per ml. Induction was performed at an A600 of 0.6 with 1 mM IPTG for four hours. After centrifugation at 5000 g for ten minutes at 4° C., the bacteria pellet was washed in a 1/20 culture volume of STE (50 mM Tris-HCl (pH 8.0), 150 mM NaCl) and stored at 270° C. Cells were resuspended in B-Per (bacterial protein extraction reagent; PIERCE) at 10 ml/g of wet weight of cell pellet and Benzonase (Novagen) was added at 125 i.u. per gram of cells. After incubation at room temperature for 30 minutes, inclusion bodies were separated from the soluble proteins by centrifugation at 20,000 g for 30 minutes, resuspended in 100 ml 1:10 diluted B-PER reagent and precipitated. This washing step was repeated 5 times.

hrPrP was extracted from inclusion bodies with 8 M urea. The extract was centrifuged and the soluble fraction was dialyzed against 0.01 M Mops buffer (pH 7.5). Approximately 85% of hrPrP precipitated during dialysis. The pellet was solubilized in 30 ml buffer (5% SDS, 0.05 M Mops (pH 7.5), 5 mM DTT) and centrifuged using a Centripore-membrane (MW 30000, PALL). SDS was removed by dialysis against 0.1 M Mops buffer (pH 7.5), 5 mM DTT. Purified protein was analyzed by SDS-PAGE in the presence of 20 mM DTT and demonstrated a molecular mass of 23 kDa with 0.85% purity.

Preparation of Brain Homogenates

One gram of brain tissue from wild-type mice (CD1BL), PrP knockout mice (PrP0/0), hamsters (Syrian golden), rats (Wistar), and scrapie-infected mice (Strain ME7) were homogenized in nine volumes of 25 mM Tris-HCl (pH 7.6), 10% (v/v) glycerol, 1 mM EGTA, 1 mM DTT, 0.32 M (NH4)2SO4 supplemented by 0.5% Nonidet P-40, 0.5% deoxycholic acid, and a cocktail of protease inhibitors (Sigma). After centrifugation (100,000 g for 30 minutes), the supernatants were aliquoted and stored at 280° C. The concentration of proteins was measured by a Bradford assay (Pierce).

RNA/Protein Gel-Retardation Assays

RNA/protein binding assays were performed in 20 ml reactions in RNA Binding Buffer (50 mM Mops (pH 7.4), 5 mM MgCl₂; 50 mM LiCl; 1 mM EGTA; 1 mM DTT; 50 ng/ml tRNA; 10% BCS; 0.05% DOX/NP-40). The concentrations of RNA, purified PrP and proteins from the cell extract vary by assay and are indicated where appropriate. Binding reactions were incubated for 20 minutes at room temperature and then separated on 7 M urea/6% polyacrylamide gels in supplemented TBE buffer (50 mM Tris-borate (pH 8.3), 1 mM EDTA, 0.05% DOX/NP-40). Gels were dried and exposed to X-ray film or analyzed by phosphorimaging (Storm 820 Phosphor Imager, Molecular Dynamics).

Supershift Assay

³²P-labeled RQ11+12 RNA of 16 fmol and 1 mg of mouse brain homogenate were incubated in RNA Binding Buffer for 20 minutes at room temperature. mAb was added to the reaction mixture and the samples were rotated for 30 minutes at room temperature. The reaction was terminated by addition of loading buffer and then analyzed on a 4 M urea/4% PA gel. Gels were dried and analyzed by phosphorimaging.

Proteinase K Resistance Assay

Resistance of PrP was analyzed by treatment of the RNA/PrP complex with Proteinase K (Sigma). The amounts of RNA and hrPrP vary by assay and are as indicated in the text. Complex formation was achieved as above, in the RNA/protein gel-retardation assays, with the length of incubation time as indicated in the text. Reactions were then adjusted to 100 mg/ml Proteinase K and incubated for an additional 30 minutes at 37° C., followed by termination with 20 ml of 2× sample buffer (Novex) containing 10 mM of PMSF and treated for ten minutes at 95° C. Protein electrophoresis on SDS/4-20% (w/v) gradient polyacrylamide gels was performed according to the protocol provided by the vendor (Novex). The gel was electroblotted onto a nitrocellulose membrane (Schliecher & Schuell) and developed in the presence of the mAb 3F-4 with the enhanced ECL kit (PIERCE).

RNase A Protection Assay

Twenty microlitre reactions of 500 pM RNA and 20 nM hrPrP (when present) were incubated for 30 minutes at 37° C. in 10 mM Tris-HCl (pH 7.4), 100 mM NaCl, 1 mM EDTA. The reactions were then adjusted to 1 pg/ml RNase A where indicated. After an additional 15 minutes at 37° C., an equal volume of loading buffer was added (94% formamide, 40 mM EDTA, 0.001% bromophenol blue/xylene cyanol), the samples heated to 95° C. for three minutes, and then immediately separated on a 6% PA/7 M urea/TBE denaturing gel.

TABLE 7

RNAs used in this study

| | |
|---|---|
| RNA | Sequence |

| | |
|---|---|
| MNV (86 nt) | GGGUUCAUAGCCUAUUCGGCUUUUAAAGGACCUUU |
| | UUCCCUCGCGUAGCUAGCUACGCGAGGUGACCCCCC |
| | GAAGGGGGGUGCCCC |
| MNVAP-1 (130 | GGGUUCAUAGCCUAUUCGGCUUCGCGCAUGGGAAU |
| nt) | UUGAGGGACGAUGGGGAAGUGGGAGCGCGUUUUAA |
| | AGGACCUUUUUCCCUCGCGUAGCUAGCUACGCGAG |
| | GUGACCCCCCGA AGGGGGGUGCCCC |
| RQ11 + 12 (197 | GGGGUUUCCAACCGGAAUUUGAGGGAUGCCUAGGC |
| nt) | AUCCCCCGUGCGUCCCUUUACGAGGGAUUGUCGAC |
| | UCUAGUCGACGUCUGGGCGAAAAAUGUACGAGAGG |
| | ACCUUUUCGGUACAGACGGUACCUGAGGGAUGCCU |
| | AGGCAUCCCCGCGCGCCGGUUUCGGACCUCCAGUG |
| | CGUGUUACCGCACUGUCGACCC |
| BS1577 (56 nt) | GGGCCCCGUAACUUCGGGAGAAGGGGUGCUCUGUU |
| | AGGGUGCAAGCCCGAGAGAGC |
| Sc-2 (112 nt) | GGAGCUCAGCCUUCACUGCGGCAAAGGCGGGAAAG |
| | CGUGCUAACGUGGAAAGCUACUCCCACGUUGUACG |
| | CGUCGCAGAUCAUUGAGUGAGGGGCACCACGGUCG |
| | GAUCCUC |
| Sc-4 (111 nt) | GGAGCUCAGCCUUCACUGCGGCAAAGGCGGGAAGC |
| | GUGCUAACGUGGAAAGCUACUCCCACGUUGUACGC |
| | GUCGCAGAUCAUUGAGUGAGGGGCACCACGGUCGG |
| | AUCCUC |

Nucleotide sequences for all RNAs are displayed in the 5′-3′ orientation. Sc-2 and Sc-4 are identical except for the deletion of adenosine 34 of Sc-2 (underlined above).

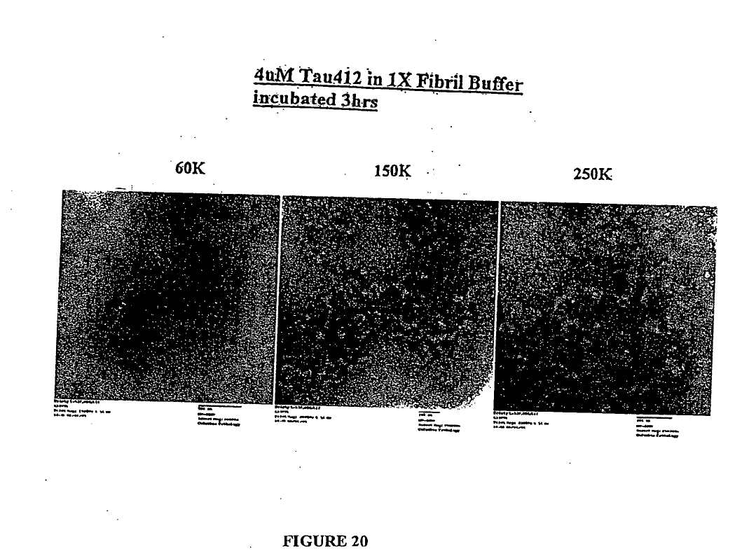

This work characterizes the interactions between different forms of PrP from various sources and several RNA constructs. The examples below reveal a number of RNA-binding characteristics for hrPrP that suggest possible functions for RNA in prion biochemistry. hrPrP demonstrated the ability to bind to individual RNA species with high affinity (Kd<100 nM), regardless of sequence or predicted secondary structure, provided that no additional RNA species were present. We term this non-specific binding. In contrast, the term specific binding describes the binding activity of hrPrP to the few RNAs (RQ11+12, AP1 and MNV:AP1), whose affinity was relatively unchanged in the presence of excess competitor RNAs. The data suggest that PrP possesses two discrete RNA-binding activities: a non-specific activity in the N-terminus (between amino acids 23 and 90) and a specific activity in the core of the protein. At least in the case of hrPrP, non-specific binding appears to be a prerequisite to specific binding because RQ11+12 does not bind to truncated hrPrP lacking the N-terminal amino acids 23-90 (PrPCterm; FIG. 20B). The effect of the N-terminal sequence may be kinetic. Just as DNA-binding regulatory proteins first bind DNA non-specifically and then rapidly translocate across the DNA to a specific binding site, the Nterminus of hrPrP binds RNA non-specifically, rapidly bringing the bound RNA into close proximity to the proposed binding site in the core of the protein. The Examples below show the conditions under which RQ11+12 can induce proteinase K-resistance in hrPrP at physiological pH—showing that after RQ11+12 binds to the N-terminus, a structural change occurs in hrPrP. This model is supported by the observation that N-terminal amino acids can influence the conformation of the C-terminal domain of PrP and that RQAAA can bind to PrPSc, which has had the N-terminus removed by proteinase K digestion, and which has a different conformation (β-sheet-rich) from hrPrP. Presumably, the binding site in the core of the protein is available in the PrPSc conformation.

It is likely that elements possessed by RQ11+12 and AP1 allow the formation of additional contacts with hrPrP that lead to specific binding. While RQ11+12 and AP1 have no obvious similarities, both RNAs are predicted to contain non-Watson-Crick base pairs. These base pairs in RQ11+12 are in the stem that differentiates it from the weaker-binding RNA, RQT157 (FIGS. 19F and 19G, Table 7). In addition to providing increased thermodynamic stability, non-canonical base pairs can alter the three-dimensional structure of an RNA by widening the major groove, enabling interactions with extended protein domains that may play a role in the specificity of protein-RNA binding. The double-stranded stem of the RNA might act as a scaffold for the binding of multiple PrP molecules, as has been proposed for double-stranded-DNA-PrP complexes formed in vitro. The ability of RQ11+12 to bind more than a single PrP peptide is demonstrated by the column cartridge in FIG. 21(C), where the stoichiometry of binding is greater than 2:1.

Early detection of prion diseases is the key issue in controlling the disease, and new assays like the luminescence immunoassay, which can detect PrP in solution at a 1 pg/ml, are very promising. Another technique, used to monitor plasma processing, uses a special protocol for the preparation of sample material followed by Western blot analysis and can reportedly detect as little as ˜2500 infectious units/ml. If these techniques were combined with the RNA-based PrP-column concentrator, which can increase the level of detection by 1000-fold, it is conceivable that as little as 3 infectious units/ml could be detected. A potential limitation of this approach is that it requires a large volume of sample (=10 ml). However, recent studies have shown that PrPRes, an isoform that is resistant to proteinase K digestion, is present in the urine of prion-infected animals. Although it is difficult to obtain, urine collected from captive animals may represent a good source of sample material because it is non-invasive, can be collected in large volumes and is suitable for the RQ11+12 RNA column described hereinbelow.

Saliva is another biological fluid that may be suitable for detection of transmissible spongiform encephalopathies because prion has been detected in the tonsils of sheep and deer. The application of RNA based affinity concentration technology during sample preparation may prove to be very useful in the development of pre-mortem prion diagnostics.

The disclosed invention is far reaching and can be applied to situations where RNA is known to specifically bind a protein. In addition, RNA constructs that can recognize specific protein epitopes and be amplified by Q-β replicase, such as RQ11+12, represent a major advance in diagnostics, combining the specificity of traditional immunodiagnostics with the sensitivity of nucleic acid-amplification-based diagnostics.

Identification of Alpha-2-Macroglobulin as a Non-NA Chaperone

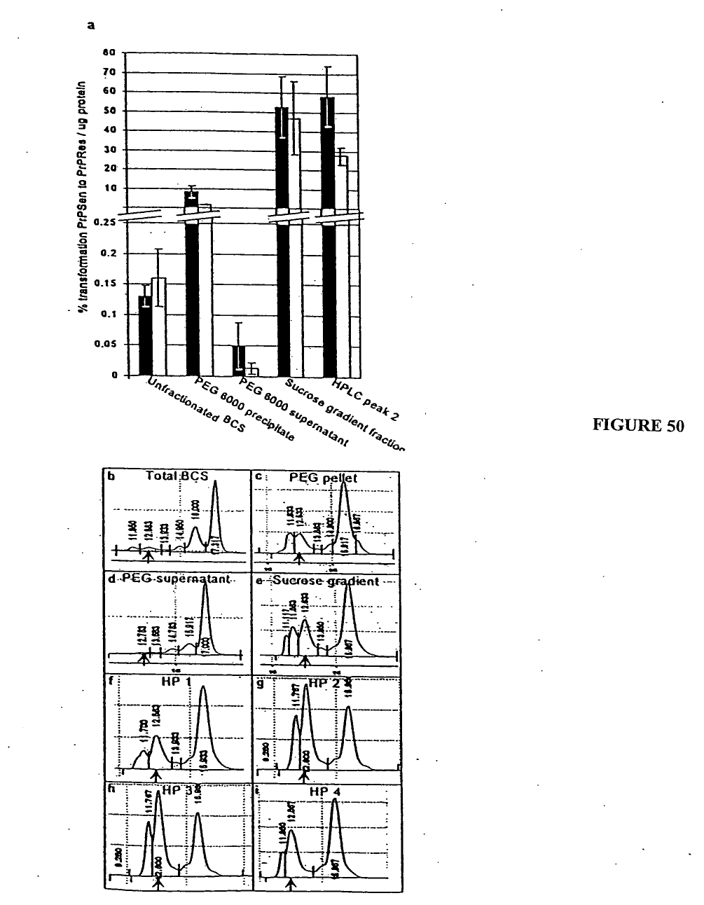

To rapidly and quantitatively assess Q-Factor activity (ability to facilitate transformation of PrPSen to PrPRes in the absence of PrPSc at physiological pH), the in vitro transformation assay was modified by labeling recombinant human PrPSen (rhuPrPSen) with fluorescent dye Cy5 for quantitative assessment of transformation in a 96-well plate format and for in-gel visualization of rhuPrPRes. Using this assay, Q-Factor from bovine calf serum (BCS) or human plasma (HP) facilitating transformation was tracked during steps of purification (FIG. 50a). Briefly, Q-Factor was concentrated in polyethylene glycol 6000 (PEG 6000) precipitate which in turn was subjected to sucrose gradient centrifugation. HPLC was applied to the sucrose gradient fraction containing the most Q-Factor. Three peaks were resolved by HPLC; the second peak, eluted at about 12 minutes, contained the majority of Q-Factor. HPLC was used to follow the enrichment of proteins in peak 2 during the steps of Q-Factor isolation (FIG. 50b-e). The absence of QFactor activity in the PEG 6000 supernatant (FIG. 50a) parallels the absence of the second peak of the HPLC trace of this fraction (FIG. 50d). The purification procedure was capable of concentrating Q-Factor approximately 500-fold.

There were large differences in the proportion of peak 2 among human plasma samples (FIG. 50f-i). However, in these samples the size of the peak did not correlate with activity suggesting that additional proteins other than Q-Factor were in this peak. For example, HP specimen 1 had high activity (42.0+0.63%) but a small amount of protein in peak 2 (FIG. 50f), whereas specimen 2 had two-fold lower activity (15.5+0.63%) but a large amount of protein in peak 2 (FIG. 50g).

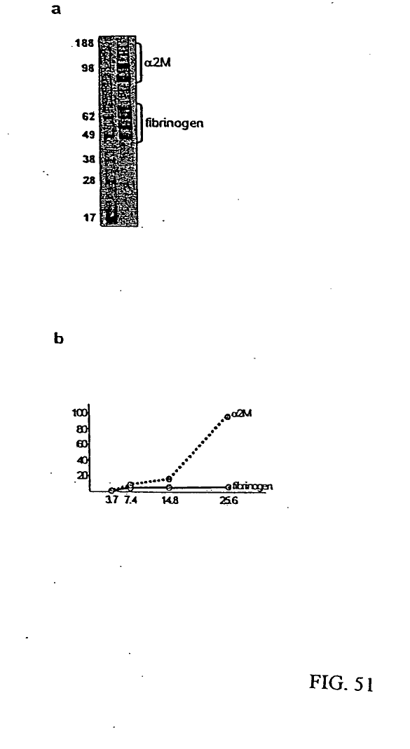

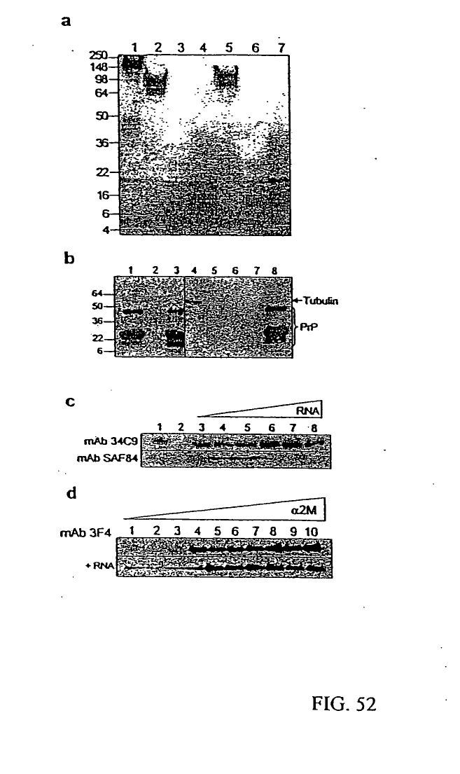

To identify Q-Factor, proteins from peak 2 were resolved by SDS PAGE for MALDITOF spectrometry. The major upper bands (˜80-180 kDa) were identified as α2M, and the lower major bands (40-60 kDa) were identified as fibrinogen (FIG. 51a). Purified preparations of α2M and fibrinogen were tested in the transformation assay. α2M, not fibrinogen, showed dose dependent facilitation of PrPsen to PrPRes transformation, thus, identifying Q-factor as α2M (FIG. 51b).

Although α2M can inhibit most endoproteinases, there is no published data regarding inhibition of PK by α2M. Therefore, the effect of α2M on PK activity was tested to determine whether the results of PrP transformation assays were simply due to inhibition of PK activity by α2M (FIG. 52a). α2M (lane 1), apo-transferrin (lane 2) and HSP90 (lane 5) were visualized before and after treatment with PK. PK treatment caused full degradation of apo-transferrin (lane 3) and HSP90 (lane 6) even in the presence of 1 mg/ml α2M (lanes 4 and 7); α2M itself was degraded, indicating that PK activity was not inhibited by α2M.

Specificity of α2M in protecting PrP from PK activity was demonstrated with rhuPrP and tubulin (FIG. 52b). PrP (lane 1) was degraded by PK (lane 2), but was protected by 4 ug α2M (lane 3), whereas tubulin (lane 4) was degraded by PK (lane 5) even in the presence of 4 or 16 ug of α2M (lanes 5, 6, respectively). Furthermore, PK treatment of a mixture of PrP, tubulin and 16 ug α2M led to the selective protection of PrP (lane 8).

rhuPrP was detected in two major bands at about 24 and 48 kDa which represent the monomer and dimer proteins. A small amount of degradation product was also present just below the 24 kDa band (lane 1). Following PK treatment PrP accumulated in a band about 18 kDa (lanes 3, 8), indicative of the PrPRes isoform, in which about 90 N-terminal residues are PK-sensitive, suggesting that α2M not only protects PrPSen from degradation but also chaperones PrPSen to the PrPRes isoform.

These experiments were performed in the absence of RQ11+12 RNA indicating that, unlike un-fractionated BCS, purified α2M was able to protect PrP from PK. To determine whether RNA modulates α2M-mediated protection increasing amounts of RNA were added to PrP transformation assays containing a constant level of α2M (FIG. 52c). An immunoblot performed with mAb 34C9 demonstrated that hurPrP (lane 1) was degraded by PK (lane 2) but the addition of 5 ug α2M, by itself, protected PrP from proteolysis (lane 3). Interestingly, there was a dose-dependent increase in signal with increasing RNA (lanes 4-8). A duplicate immunoblot performed with mAb SAF84, which normally has no specificity for human PrP (lane 1), was able to interact with rhuPrP in the presence of 2 or 4 pmol RQ11+12 RNA and 5 ug α2M (lanes 4 and 5). The RNA-dependent changes in the qualitative and quantitative recognition of PrP by these antibodies suggested that these molecules chaperoned conformational alterations in PrP effecting antibody recognition.

Dose dependence of α2M for protection of rhuPrP was determined in the absence or presence of 8 pmol RQ11+12 RNA (FIG. 52d). A minimum of 6 ug of α2M was necessary to protect PrP from degradation in this assay (lane 4). The addition of RNA afforded a small amount of protection at minimal α2M levels (lanes 1-3) demonstrating that these molecules can function in concert to facilitate PrP transformation to PrPRes. Interestingly, the combination of RNA and 6 ug α2M facilitated less protection than 6 ug α2M alone (lane 4). Taken together these results suggest that RNA and α2M appear to chaperone conformational changes in PrP that are dependent on the stochiometry of the components.

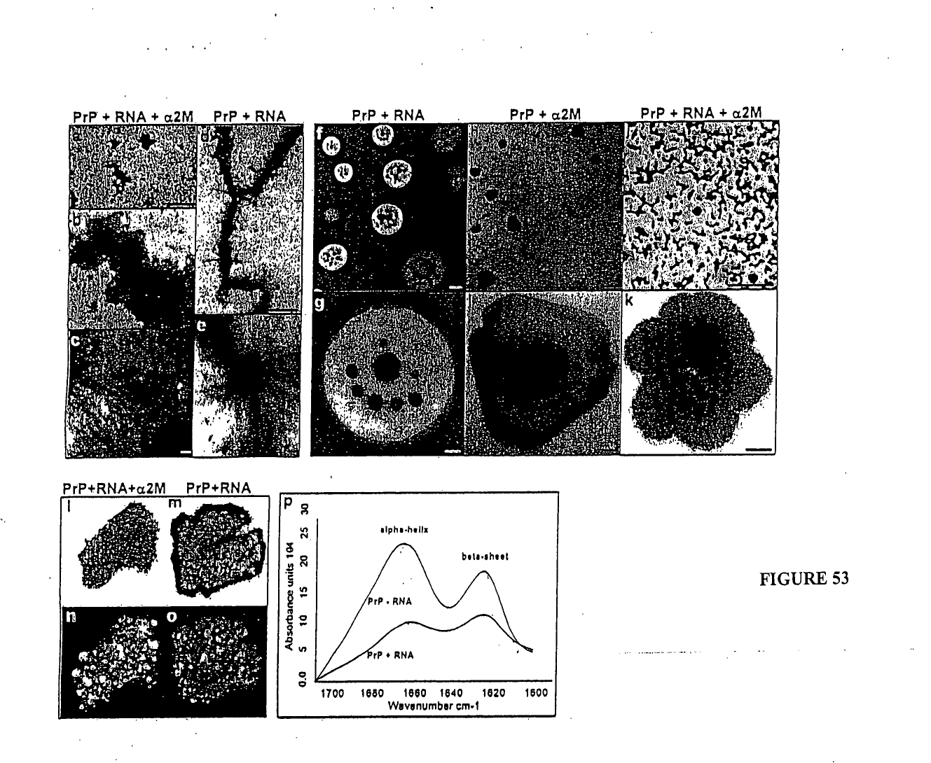

Negative stain electron microscopy was used to characterize the structure of complexes formed by rhuPrP-Cy5, RQ11+12 RNA and α2M (FIG. 53a-c). Incubation of rhuPrP-Cy5, RQ11+12 RNA and α2-macroglobulin (1 mg/ml) led to the formation of aggregates as surrounding material was incorporated into complexes over the course of one hour. Fibril-like structures were visible within these compact aggregates (FIG. 53c). Co-incubation of rhuPrP-Cy5 and RQ11+12 RNA for 10 minutes led to the rapid formation of large, strand-like structures exhibiting a filamentous substructure (FIGS. 53d, e).

In contrast, longer incubation (12 hrs at 37° C.) of unlableled rhuPrP led to the formation of structures with different organizations depending on the chaperone coincubated with rhuPrP (FIG. 53f-k). Incubation of rhuPrP with RNA, in the absence of α2M, led to the formation of spherical structures (spherules). The diameters of the spherules ranged from about 500 nm to 5 microns, and their surfaces appeared to be at the interface of two separate liquid phases (FIGS. 53f, g). Multiple solid particles of varying size, number and electron density regularly formed inside the spheres. In the absence of RNA, rhuPrP and higher levels of α2M (1.5 mg/ml) led to formation of triangular crystals about 1 micron in diameter (FIGS. 53h, i). Co-incubation of rhuPrP with RNA and α2M (1.5 mg/ml) led to the formation of microspherulites (FIG. 53 j, k). The diameters of the individual microspherulites were about 100 nm. Individual microspherulites also clustered to form aggregates of a range of sizes.

Congo red was used to determine whether PrP complexes formed had amyloid properties. PrP transformation assays were performed with 2 pmol RQ11+12 RNA with or without 10 ug α2M and incubated for 30 min r.t. followed by 30 min at 37° C. Aggregated material appeared salmon-red using bright field microscopy (FIGS. 53l, m) and displayed apple-green birefringence using crossed polarizing filters indicative of amyloid structure (FIGS. 53n, o). PrP-RNA material had birefringence that was mostly punctate (FIG. 53n), whereas PrP-RNA-α2M material had brighter overall birefringence (FIG. 53o) suggesting that α2M facilitated an additional conformational shift toward anti-parallel β-sheet structure. When the stochiometry of the components and the incubation time and temperature were changed (15 ug α2M, 12.5 hours at 37° C.) the aggregated material was stained by Congo red but did not show birefringence.

Fourier-Transform Infrared Spectroscopy (FTIR) was performed on rhuPrP and on rhuPrP in complex with RQ11+12 RNA to observe structural changes in PrP. The proportion of α-helical to β-sheet structure was altered by interaction of PrP with RNA (FIG. 53p). Free rhuPrP had mostly α-helical structure (red curve), whereas rhuPrP in complex with RNA had mostly β-sheet structure (blue curve) indicating that RNA chaperoned a shift in PrP structure toward a PrPRes isoform, characteristically rich in β-sheet structure.

Herein we describe the identification of “Protein X” as α2M, an abundant plasma protein. Formation of PrPRes was not caused by inhibition of PK by α2M, rather α2M selectively protected PrP from PK. Formation of aggregates such as crystals may help explain the acquisition of PK-resistance, although the formation of aggregates of PrP with RNA does not confer PK-resistance to PrP, suggesting a more subtle mechanism such as conformational change to PrPRes. Both RNA and unpurified Q-Factor were necessary for PrP transformation, whereas purified α2M was necessary and sufficient for PrP transformation suggesting the interplay of multiple factors in biologically complex serum. Nevertheless, RNA plays a significant chaperone role in changing PrP-α2M structures from crystals to spherulites and in altering antibody recognition of PrP. RNA by itself chaperones PrP to a conformation with increased β-sheet structure and facilitates the formation of spherules reminiscent of viral particles in their organization. DNA and PrP at acidic pH were also reported to form spherical amyloids but were much smaller than our spherules and did not have inclusions. Together, these observations suggest that PrP may encapsulate NA adding to PrP's biochemical characteristics of a retroviral nucleocapsid protein. However, there is no evidence that PrP-NA complexes form such structures in vivo. The stochiometry of PrP, RNA and α2M was critical for the determination of the structures of the complexes formed and may have biological significance as plasma levels of α2M vary with age, sex and disease. Co-incubation of PrP, RNA and α2M led to the formation of aggregates containing fibril-like structures and behaved like amyloid when stained with Congo red. However, an increase from 1.0 to 1.5 mg/ml of α2M led to the formation of microspherulites which stained with Congo red but did not exhibit birefringence indicative of amyloid.

Exceptional aspects of this in vitro PrPSen to PrPRes transformation system are its physiological pH and components, full length PrP, an RNA chaperone, an extracellular protein chaperone and no added PrPSc. This system may uniquely serve as a model for spontaneous generation of PrPSc in blood. Biological analysis of the association of PrP with α2M may provide insight into the mechanisms of distribution of PrPSc during disease progression and may provide a pathway for transport of PrP-α2M via the LDL receptor-like protein into acidic endosomes that may further contribute to PrP transformation. The association of PrP with α2M may also have implications for developing methods to remove PrP from blood products. Biological involvement of α2M has already been found in Alzheimer disease, a common amyloid disorder. α2M is genetically associated with Alzheimer's disease and interacts with β-amyloid to influence fibril formation and its metabolism.

Methods

Preparation of RNA and rhuPrP and Cy5 Labeling

RQ11+12 RNA and hrPrP were prepared as previously described. The refolding of hurPrP and removal of SDS were achieved by gradual dialysis of the sample against 1%, 0.5%, 0.1% and 0.01% and 0% SDS in 50 mM MOPS, pH 7.5, 1 mM DTT buffer. Fluorescent molecule Cy5 (Amersham) was used to label refolded and purified hurPrP.

In vitro Transformation Assay with rhuPrP-Cy5

1 ug rhuPrP was incubated with 2 pmol RQ11+12RNA and 4 ul unfractionated BCS or HP or fractions from the Q-Factor purification process (see below) in 10 ul buffer (50 mM MOPS, pH 7.4, 5 mM MgCl₂, 50 mM LiCl, 1 mM DTT, 0.05% sodium deoxycholate, 0.05% NP-40). The reaction mixture was incubated 30 minutes at room temperature and 30 min at 37° C., treated with 25 ug/ml proteinase K (PK) for 30 min at 37° C., and the PK was inactivated by the addition of 10 mM PMSF. The reaction mixture was boiled for 5 minutes in Laemmli sample buffer (Sigma) and run on 4-20% polyacrylamide gradient gels (Gradipore). PrPRes was visualized directly in gels using a FluorImager (Molecular Dynamics) at an excitation wavelength of 544 nm and an emission wavelength of 595 nm; alternatively PrPRes aggregates were detected directly after brief centrifugation. Pellets were washed with 100 ul water, suspended in 10 ul 1M Tris-HCl pH 10 and diluted with 50 ul 1% SDS/50 mM Tris-HCl pH 7.5. Suspended pellets were transferred to a 96-well micro-titer plate and fluorescence was determined using a FluoroskanII (Labsystems) with an excitation wavelength of 544 nm and an emission wavelength of 590 nm.

Purification of O-Factor from BCS and HP

To concentrate Q-factor by precipitation 5 ml of BCS (Sigma) or HP (New York Blood Bank) were mixed with 1 ml of 45% PEG 6000 (Sigma), incubated for 15 minutes at room temperature, and pelleted by centrifugation at 350×g for 15 minutes. The high molecular weight debris was washed from the top of the pellet without disturbing the pellet with 2 ml of saline and suspended in 5 ml 0.1% EDTA in 0.9% saline. Protein was precipitated with 1 ml 45% PEG 6000, centrifuged and washed again. The second precipitate was dissolved in 1 ml saline, and PEG 6000 was removed by dialysis against EDTA/saline solution. Q-factor was further purified by centrifugation through a linear sucrose gradient (26-60% (w/v) in a total volume of 36 ml at 360,000 g for 18 hrs at 4° C. using a VTi-50 rotor (Beckman). The gradient was divided into 20, 1.8 ml fractions and 4 ul of each fraction was assayed for PrP transformation activity. Four fractions with the highest activity were combined and dialyzed overnight against buffer. High pressure size exclusion chromatography (HPLC) was performed at 23° C. using a rate of 1 ml/min on a 600 mm×7.80 mm BioSep-SEC-S 3000 HPLC gel filtration column equilibrated in running buffer (50 mM Tris-HCl, pH 7.5; 0.1M LiCl). Fractions were concentrated individually using Microcon YM-10 Centrifugal Filter Devices (Millipore), and immediately assayed for transformation activity using 4 ul of each fraction.

Protein Identification

Proteins isolated by HPLC at Q-RNA, Inc. were resolved in a 4-12% gradient gel (Invitrogen). Six major individual protein species were analyzed from peak 2. Protein identification was performed by the Roche Protein Expression Group using standard MALDI-MS technology. MALDI-TOF data for each band was searched against a protein sequence database using program “Knexus”.

Immunoblots

mAbs 34C9 (Prionics), 3F4 (Signet) and SAF84 (Cayman Biochemicals) were used for detection of hurPrP in standard immunoblot assays.

Negative Stain Electron Microscopy

A 10 ul drop of sample was transferred onto 400-mesh formvar-carbon-coated copper grids, stained with a drop of 2% uranyl acetate and rinsed with water. A JEOL 1200 EX II Electron Microscope was used at 80 kV and images were captured with a Hamamatsu XR60 digital camera.

Congo Red Staining and Polarized Light Microscopy

One ug rhuPrP was incubated with 2 pmol RQ11+12 RNA+/−1 mg/ml α2M for 30 min r.t. and 30 min at 37° C. in buffer. Congo red was added to a final concentration of 10 uM and incubated for 30 min r.t. Aggregated material was pelleted, washed with buffer, and observed at 640× magnification.

FTIR

Interferograms were recorded between 1700 and 1600 cm-1 and X spectra were averaged. They were acquired in transition mode using CaF2 cell separated by spacer of 25 um. After a reference spectrum of the instrument and fresh D2O lot that was used was recorded, the complex or its individual component solutions were applied, and the absorbance spectrum of the sample were measured. To facilitate comparison, the spectra were normalized with respect to their maxima. Typical concentrations for rhuPrP and its complexes were 70-600 uM depending on specimen concentration and its nature.

### Transgenic Animals

Current transgenic mouse models of Alzheimer's, Parkinson's and other related disorders are genetically programmed to express corresponding human proteins and to simulate motor deficits and brain alterations found in these diseases. They were used in studies of human neurological disorders as well as for drug discoveries and validation. However, an important characteristic of traditional transgenic animal models is that the disease is a result of over-expression of the human protein. The transgenic animal model described herein develops a neurological condition in a more natural way, without introduction of the foreigner protein and without over-expression of this protein. Expression of RQ11+12 RNA is induced and the effect of the synthesized RNA on aggregate formation in the brain and other mouse tissues can be validated using standard cytological, molecular biology and biochemical methods.

### “Q-Cartridges”

A Q-Cartridge is a disposable cartridge with a cellulose matrix that is coated with a prion-specific RNA ligand. This RNA ligand has high affinity for various configurations of protein prions (PrP) including the cellular (PrPc) and pathogenic (PrPSc) forms. Q-Cartridge has the ability to concentrate prion protein from biological specimens including fluids (e.g., blood). Together with I-Q-Amp, the present invention also, therefore, includes diagnostics technology for diseases associated with misfolding of proteins.

### Nucleic Acids Bind Protein Targets

The NA ligands, also referred to herein as “amplibodies” or “NA antibodies” (mentioned above), of the present invention can be either DNA or RNA molecules that comprises a nucleotide sequence required to interact with a particular target molecule. The target molecule can include a protein or protein fragment. In preferred embodiments, the NA compositions of the present invention perform like antibodies whose affinity is for one or more agglomeration proteins. These NA antibodies can be directed at any number of targets. In one embodiment of the invention these NA antibodies are directed to amyloid or agglomeration proteins. More specifically, an embodiment encompasses NA antibodies directed towards prion proteins involved in neurological diseases, such as spongiform enchephalitis. (ee for example, PCT/US02/16922, filed May 30, 2002, the entire teachings of which is incorporated herein by reference.) Nucleic acid antibodies can comprise artificial, synthetic nucleotide sequences that a skilled practitioner can insert by methods well known in the art. Therefore, amplibodies can also be chimeric NA molecules that are designed specifically for a particular target.

Nucleic acid antibodies can be seen as universal detector molecules. Nucleic acids are easily modified for signal detection, whereas monoclonal antibodies require an additional enzyme-based component to produce a signal. The universal nature of NA antibodies allow for detection of both non-nucleic acid molecules as well as for hybridization with nucleic acid targets. The universe of non-NA targets includes proteins, toxins, and small bioregulators against which antibodies are practically impossible to develop.

The degree of molecular discrimination achieved by a NA antibody in recognition of the corresponding target matches and, in some instances, surpasses that of traditional monoclonal antibodies. An important advantage of amplibodies and in contrast to traditional monoclonal antibodies, NA antibodies can be easily modified to increase their avidity to targets and enhance their specificity. Thus, the NA antibodies disclosed herein represent new and powerful molecular biological tools with potentially wide applications in medicinal diagnosis, biotechnology and therapeutics. Nucleic acid antibodies can be used for detection of designated targets in formats that employ existing immunodiagnostic or nucleic acid assay hardware. Importantly, they can be used together with traditional antibodies to dramatically increase the total affinity of extant immunodiagnostic assays.

The role of NA antibodies in the discovery of new drugs, the design of diagnostic devices and therapeutics, as well as in basic and applied biotechnology research, is enormous. The unique properties of the presently described NA antibodies, with their specific and tight binding activities, create new opportunities to construct extremely powerful molecular biological tools that can be applied to various practical purposes, as well as further our understanding of biological processes.

In one aspect of the present invention, the NA antibody is an RNA molecule. RNAs are unique molecules in the universe of macromolecules; they combine features possessed by both DNA and proteins. RNA, like DNA, is a nucleic acid, but like proteins, RNA can fold into a variety of stable secondary structures that in a “lock and key” fashion can interact and form complexes with complimentary structures from other molecules. It has been shown, without any known exception, that the specific interaction of RNAs with non-NA targets is determined by their nucleotide composition and by the shape of the binding regions of both the RNA and target molecules. Typically, target proteins have RNA-binding motifs that will receive the proper RNA secondary structure.

Nucleic acid antibodies of the present invention comprise one or more RNA or DNA molecules having affinity for at least one protein involved in protein agglomeration. The NA component is a naturally or non-naturally occurring molecule with twenty or more nucleotide bases. In one embodiment, at least one nucleotide sequence portion of this RNA molecule has affinity to at least one consensus sequence present in the agglomeration RNA-binding protein. A “consensus sequence” of the present invention refers to an RNA-binding motif present in a protein that recognizes single-stranded RNA secondary structural elements such as hairpin loops, bulge loops, internal loops, or single-stranded regions. In one embodiment of the present invention, the portion of RNA polynucleotide having affinity for agglomeration proteins is a sequence that is derived from either an RNA virus, an RNA phage, a messenger RNA (“mRNA”), a ribosomal RNA (“rRNA”), a transfer RNA (“tRNA”), a sequence that is received as a template by one or more RNA dependent RNA polymerases, or a combination thereof.

In some embodiments of the present invention, the RNA has one or more loops or bulges characterized by non-Watson-Crick pairing. The non-Watson-Crick pairing refers to regions of an otherwise normal Watson-Crick base pairing, there is a region in which the nucleotides are not paired or the pair is not the conventional Watson-Crick base pairing. For example, the nucleotides are held in close proximity with a like nucleotide (e.g., purine-purine or pyrimidine-pyrimidine). A preferred bulge or loop has a grouping of guanine nucleotides in series, for example, in a quartet. This guanine feature often corresponds to a recognition sequence in an RNA pol. For example RQ11+12 comprises a sequence that is derived from the Rev protein binding site and a sarcin recognition site that presents a series of guanine nucleotides. See Iwai, S, et al., NAR 20(24): 6465-6472, 1992; the teachings of which are incorporated herein by reference.

There is no absolute length requirement for participating polynucleotide sequences. However, a preferred range is from about 20 to about 10,000 nucleotides. One of ordinary skill in the art will be able to determine the appropriate length of nucleotide sequence to employ for the RNA amplibodies of the present invention. It should also be understood that the polynucleotide sequences of the instant invention can be embedded within longer strands of nucleic acids or associated with other molecules.

It is understood that complementary base-pairing of individual base pairs generally follows Chargaff's Rule wherein an adenine pairs with an uracil (or thymine if DNA) and guanine pairs with cytosine. However, there are modified bases that account for unconventional base-pairing. A modified nucleic acid is understood to mean herein a DNA or RNA nucleic acid molecule that contains chemically modified nucleotides. The term “nucleic acid analogue” is understood herein to denote non-nucleic acid molecules such as “RNA” and morpholino that can engage in base-pairing interactions with conventional nucleic acids. These modified bases and nucleic acid analogues are considered to be within the scope of the instant invention. For example, nucleotides containing deazaguaine and uracil bases can be used in place of guanine and thymine, respectively, to decrease the thermal stability of probes. Similarly, 5-methyl-cytosine can be substituted for cytosine in complexes if increased thermal stability is desired. Modification to the sugar moiety can also occur and is embraced by the present invention. For example, modification to the ribose sugar moiety through the addition of 2′-O-methyl groups which can be used to reduce the nuclease susceptibility of RNA molecules. Modifications occurring with different moieties of the nucleic acid backbone are also within the scope of this invention. For example, the use of methyl phosphate, methyl phosphonate or phosphorothioate linkages to remove negative charges from the phosphodiesters backbone can be used.

The inventors have conducted experiments showing multiple binding sites on the same agglomeration protein. These sites where shown to have affinity for different RNA polynucleotide compositions. The agglomeration protein studied was a prion protein (PrP), see Weiss, S. et al., J. Virol. November 1997: 71(11): 8790-7; the entire teachings of which are incorporated herein by reference. Two RNA polynucleotides were examined, one is the MNV RNA and the other is the MNV RNA with an AP1 RNA sequence (see Weiss et al.) cloned into it. The cloned MNV RNA is referred to as the PrP amplibody (“PrP-Amp”).

Under suitable conditions, RNPs can non-specifically bind to RNA having gross features, or secondary structures, that are recognized by the protein without regard to particular nucleotides or nucleotide sequences. It is generally believed that a nucleic acid's higher ordered structure is what provides binding recognition to the protein. For example, RNA is notorious for possessing secondary structures like loops that may in turn serve as structural motifs used for binding with RNPs. (This notion is amenable to analysis simply by taking a primary nucleotide sequence of an RNA molecule that binds to a particular protein and changing the nucleotide sequence of a particular secondary structure, like a loop, in order to knock-out the structure and determine the binding avidity between the mutated RNA and protein.) In certain embodiments of this invention, protein agglomeration is facilitated by such nucleic acid binding. The isolation and characterization of nucleic acids having affinity for agglomeration proteins aids provides valuable understanding and treatment of diseases involving protein agglomeration. This invention is directed toward this goal.

### Nucleic Acid Ligands also Bind Protein Targets with Specificity

Stringent in vitro assays have demonstrated that heterogeneous nuclear RNA-binding proteins (hnRNP) have different preferences for specific RNA sequences. One study examined the binding of various hnRNPs to various RNAs under 2M NaCl conditions resulting in a finding of striking avidity of hnRNPs for their preferred RNAs. These studies indicate that different hnRNP, and apparently other RNPs, discriminate among and between different RNAs. This property of discrimination can be exploited in the isolation, purification and classification of various groups of RNPs.

The molecular architecture of RNPs was studied in detailed using hnRNPs. However, the general principles gained through studying the hnRNP systems are applicable to other RNPs. Most RNPs have a modular structure with one or more RNA-binding domains (RBD) and special domains that mediate interaction with another protein. The hallmarks of RBDs in RNPs are distinct consensus sequences separated from each other by stretches of approximately thirty amino acids. Most of the amino acids that are involved in binding RNA are located in β-pleated sheets. These particular structural elements of RBDs appear to provide an exposed surface that can serve as a platform to which an RNA molecule can bind. The RNA, when bound, remains exposed (as opposed to being buried within a pocket of the protein) and thus accessible to other proteins. Many RNPs contain more than one RBD and can therefore bind to multiple RNA sequences or interact with multiple RNA molecules simultaneously.

High affinity RNAs (with Kd's ranging from 0.1-1.0 nM) have been successfully identified for a large number of targets, ranging in chemical composition and size from small organic molecules to highly complex multimeric structures, such as viruses.

### Specific Embodiments of Nucleic Acids of Present Invention

A nucleic acid molecule of the present invention, e.g., a nucleic acid molecule having the nucleotide sequence of SEQ ID NO. 1-10, or a portion thereof, can be isolated using standard molecular biology techniques and the sequence information provided herein. The nucleic acid molecule of the invention, moreover, can comprise only a portion of the nucleic acid sequence of SEQ ID NO 1-10 of the invention, or a fragment which can be used as a probe or primer. The probe/primer typically comprises substantially purified oligonucleotide. Using all or portion of the nucleic acid sequence of SEQ ID NOS. 1-10 as a hybridization probe, a molecule comprising SEQ ID NOS. 1-10 can be isolated using standard hybridization and cloning techniques as described in Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989.

In another embodiment, an isolated nucleic acid molecule of the invention comprises a nucleic acid molecule which is a complement of the nucleotide sequence of SEQ ID NOS. 1-10, or a portion thereof A nucleic acid molecule that is complementary to such a nucleotide sequence is one which is sufficiently complementary to the nucleotide sequence such that it can hybridize to the nucleotide sequence, thereby forming a stable duplex.

The nucleic acids of the present invention can be amplified using cDNA, mRNA or alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to marker nucleotide sequences, or nucleotide sequences encoding a marker of the invention can be prepared by standard synthetic techniques, e.g., using an automated DNA synthesizer.

Probes based on the nucleotide sequence of a nucleic acid molecule encoding SEQ ID NOS. 1-10 can be used to detect agglomeration proteins. In other embodiments, the probe comprises a labeling group attached thereto, e.g., the labeling group can be a radioisotope, a fluorescent compound, an enzyme, or an enzyme co-factor. Such probes can be used as a part of a diagnostic test kit for identifying cells or tissue which misexpresses, e.g., over- or under-express, a polypeptide of the invention, or which have greater or fewer copies of a gene of the invention.

The amplibodies of the present invention can be amplified using an RNA-dependent RNA polymerase. In one embodiment of the present invention, the polymerase is Q-Amp. Q-Amp is derived from Q-beta replicase. Q-beta replicase can be isolated and purified from Q-beta bacteriophage. The bacteriophage contains a plus-strand RNA. This plus-strand serves both as mRNA for viral protein synthesis and as a template for the RNA-depednent RNA polymerase, Q-beta replicase. In the presence of the plus-strand, and other cofactors, the enzyme Q-beta replicase synthesizes the complementary minus-strand RNA. The minus-strand, in turn, serves as a template for plus-strand synthesis. Q-Amp is derived from the Q-beta replicase and comprises eukaryotic elongation factor Ts (Ef-Ts), eukaryotic elongation factor Tu (Ef-Tu), S1 nuclease, and a Replicase component. (Q-Amp is available from Q-RNA, Inc, New York, N.Y.) Q-Amp recognizes specific RNA templates and can amplify them exponentially, for example up to one billion-fold, in fifteen minutes under isothermal conditions. Templates for Q-Amp can contain sequence insertions that may have specific functional applications, for example, sequences that increases the avidity between an amplibody and an agglomeration protein. It should be appreciated by those skilled in the art, that replicases other than Q-Amp and Q-beta replicase may be used and are considered to be within the scope of this invention.

### Examples of Some Functions of Certain Embodiments of the RNAs

The RNA templates of the present invention are RNAs that can be received and amplified by Q-beta replicase, Q-Amp, and nucleic acid replicases for DNA or RNA. (See PCT/US02/16922.) The skilled artisan will appreciate that any RNA dependent RNA polymerase (“RNA pol”) that will amplify the NA antibodies of the present invention are within the scope of this invention. In one aspect of this invention, these templates for the RNA pol (replicase) have at least one oligonucleotide sequence having affinity for an agglomeration protein. For example, MDV, MNV, and RQ RNA templates can have incorporated into their respective sequences a series of guanine nucleotides.

In one embodiment of the instant invention, the RNA template is selected from the group consisting of midi-varient RNA (MDV RNA), mini-varient RNA (MNV RNA), MNV-AP1 RNA, MNVUP RNA, MNVLO RNA, RQ RNA, and combinations thereof. The sequences of these RNA templates are as follows:

The RNA template is RQ11+12, the sequence of which is (SEQ ID NO 1):

5′GGGGUUUCCAACCGGAAUUUGAGGGAUGCCUAGGCAUCCCCCGUGCG
UCCCUUUACGAGGGAUUGUCGACUCUAGUCGACGUCUGGGCGAAAAAUGU
ACGAGAGGACCUUUUCGGUACAGACGGUACCUGAGGGUAUGCCUAGGCA
UCCCCCGCGCCGGUUUCGGACCUCCAGUGCGUGUUACCGCACUGUCGACCC
3′

The DNA sequence encoding MDV RNA (SEQ ID NO: 2) is:

5′GGGGACCCCCCGGAAGGGGGQACGAGGTGCGGGCACCTCGTACGGGAG
TTCGACCGTGACGAGTCACGGGCTAGCGCTTTCGCGCTCTCCCAGGTGAC
GCCTCGTGAAGAGGCGCGACCTTCGTGCGTTTCGGCGACGCACGAGAACC
GCCACGCTGCTTCGCAGCGTGGCCCCTTCGCGCAGCCCGCTGCGCGAGGTGA
CCCCCGAAGGGGGGTTCCCCA3′

The DNA sequence encoding MNV RNA (SEQ ID NO: 3) is:

5′
GGGTTCATAGCCTATTCGGCTTTTAAAGGACCTTTTTCCCTCGCGTAGCTA
GCTACGCGAGGTGACCCCCCGAAGGGGGGTGCCCC3′

The DNA sequence encoding MNV-AP1 RNA (SEQ ID NO: 4) is:

5′GGGTTCATAGCCTATTCGGCTTCGCGCATGGGAATTTGAGGGACGATGGG
GAAGTGGGAGCGCGTTTTAAAGGACCTTTTTCCCTCGCGTAGCTAGCTACGC
GAGGTGACCCCCCGAAGGGGGGTGCCCC3′

The DNA sequence encoding MNVUP RNA (SEQ ID NO: 5) is:

5′
GGGTTCATAGCCTATTCGGCTTCGCGCCCGTTTATAATACTTAGTGAGCGC
GTTTTAAAGGACCTTTTTCCCTCGCGTAGCTAGCTACGCGAGGTGACCCCC
CGAAGGGGGGTGCCCC3′

The DNA sequence encoding MNVLO RNA (SEQ ID NO: 6) is:

5′
GGGTTCATAGCCTATTCGGCTTCGCGCCCCTGGGGTTTGCCTCAGGAGCGC
GTTTTAAAGGACCTTTTTCCCTTGCGTAGCTAGCTACGCGAGGTGACCCCC
CGAAGGGGGGTGCCCC 3′

The RNA sequence can be obtained using DNA that encodes for the RNA sequence. The DNA can be inserted within a suitable vector followed by transfecting a suitable host with the vector using methods well known to those skilled in the art. The transcripts can then be isolated by methods commonly known in the art.

### Binding Affinity and Binding Sites

Table 2 below, shows that the binding of Prp-Amp is very stable, whereas the binding stability of MNV is dependent upon the presence or absence of tRNA.

TABLE 2

| | | Apparent | Apparent |
|---|---|---|---|
| Description | RNA | Kd (nM) + tRNA | Kd (nM) |
| Vector + AP1 | PrP-Amp | 12 | 4 |
| (127 nt) | | | |
| Vector (87 nt) | MNV | 1000 | 38 |

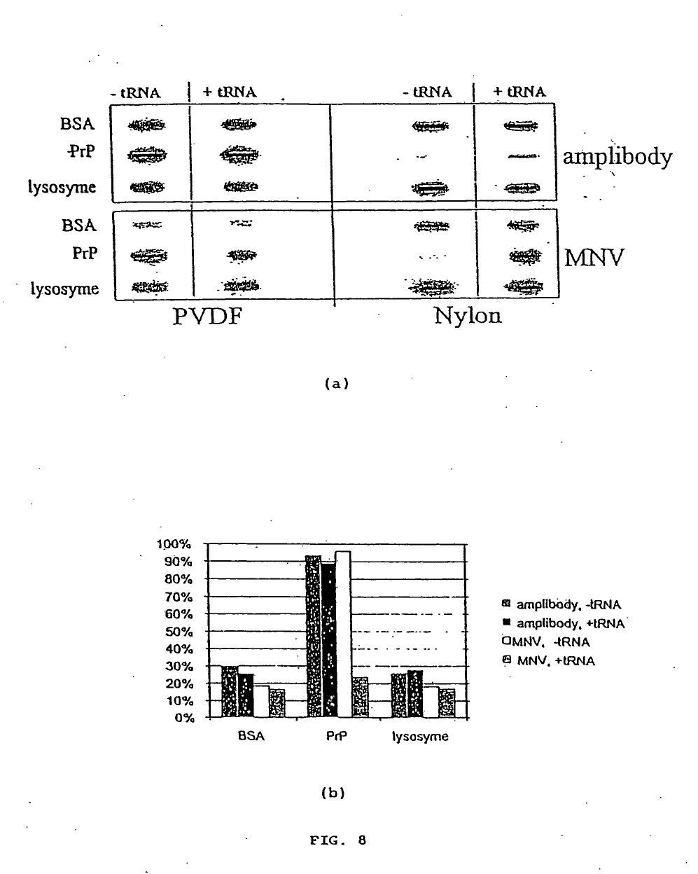

The binding data presented above suggests that there are at least two types of binding sites on the prion protein. See FIG. 9. First, there is a low affinity site where MNV binds in the absence of tRNA. Then there is the high affinity site where the PrP-Amp binds.

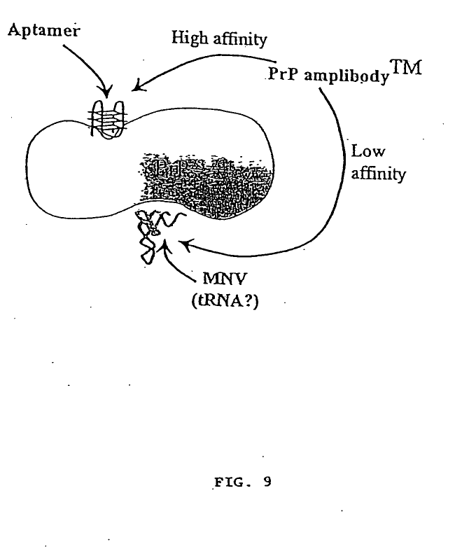

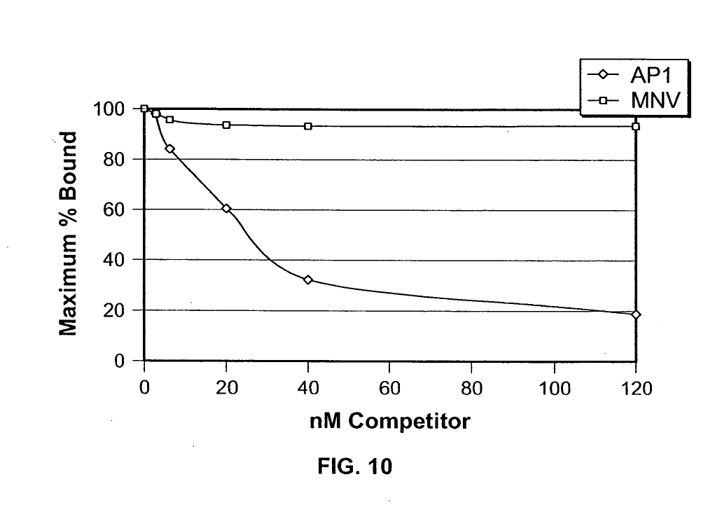

This multiple binding thesis was further examined in a competition study. The binding of PrP-Amp to the prion protein was challenged using either AP1 or MNV. FIG. 10 shows that AP1 effectively inhibits PrP-Amp binding, whereas MNV has little or no effect on PrP-Amp's binding. Clearly, then, there must be at least two types of RNA binding sites on the prion protein

Using tRNA as an instrument to discriminate between binding sites, other RNAs were examined for their binding affinity to the prion protein in the presence of tRNA. The summary of the findings is presented in Table 3.

TABLE 3

| RNA | Apparent Kd (nM) + tRNA |
|---|---|
| RQ11+12 | 98 |
| MDV | 167 |
| RQT157 | 185 |
| BS1577 | 2500 |

Based upon the favorable K_{d} value for RQ11+12, the binding to the prion protein in the presence or absence was then assessed. Table 4 presents this data. It can be appreciated by the data presented in Table 4 that the binding of RQ 11+12 to the prion protein in the absence of tRNA is about 4-fold tighter as when compared to binding in the presence of tRNA.

TABLE 4

| RNA | Apparent Kd (nM) + tRNA | Apparent Kd (nM) − tRNA |
|---|---|---|
| RQ11+12 | 98 | 26 |
| PrP-Amp | 12 | 4 |

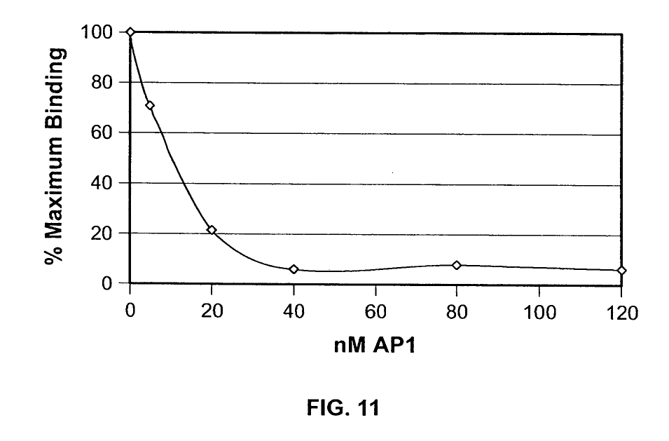

To further investigate the binding of RQ 11+12, AP1 RNA was used to compete with RQ11+12's binding to the prion protein. FIG. 11 shows that AP1 can effectively inhibit RQ11+12's binding. This data also suggests that RQ11+12 is binding to the high affinity site of the prion protein.

The data thus far presented suggests that binding to the high affinity site requires the presence of a specific RNA structural element. In order to determine the RNA structural elements for stable binding to the high affinity site (in the presence of tRNA), the inventors examined the binding of other RNAs to the prion protein. RNAs from MNV were chosen for this experiment. The MNV derivatives contained a random insert of 20 nucleotides. Table 5 presents the data obtained from the experiment. From the data, it can be appreciated that there is a large variation in binding to the high affinity site between RNAs of equal length but of different secondary structure. Presumably, variance in the structure of a single stem accounts for the difference in binding to the high affinity site. Therefore, there appears to be specific RNA structural elements that engender tight binding to the high affinity site.

TABLE 5

| RNA | Apparent Kd (nm) + tRNA |
|---|---|
| MNV-1 (MNVUP) | 217 |
| MNV-2 (MNVLO) | 1667 |
| MNV | 1000 |

### Effecting a Protein

One of the crucial pathogenic events in prion disease propagation is the structural conversion of benign, a-helices rich PrPC into the highly stable, β-sheet rich PrPSc isoform associated with infectivity. The PrPC→PrPSc conversion results in the alteration of some of the physical and biochemical traits of the protein such as a reduction in solubility and an increase in resistance to Proteinase K (PK) hydrolysis. Therefore, PrPC is often referred to as PrPSen as are recombinant forms of the protein. Conversion of PK sensitive PrP (PrPSen) into a PK resistant PrP isoform (PrPRes) is considered to be one of the indicators for transformation of PrPC into infectious PrPSc and, hence, disease progression. PrPSc isoforms aggregate into plaques, rods and scrapie-associated fibrils (SAF) that accumulate in the brains of affected animals and humans. Knockout mice lacking the prn-p gene, which encodes the PrP protein are unable to contract prion disease after inoculation with infectious material containing PrPSc, demonstrating the requirement for both PrPC and PrPSc for development of prion disease. Mice inoculated with BSE extracts can develop prion disease in the absence of detectable PrPRes, suggesting that there may be other cellular components involved in PrPC→PrPSc conversion, PrPSc aggregation, prion disease transmission and propagation.

Recently, it was found that several small, highly structured RNA species (shsRNAs) derived from a collection of artificially constructed RNAs possess high affinity for the human recombinant prion protein (hrPrP) under physiological conditions (low concentrations, 37° C., pH 7.5). The shsRNAs demonstrated a wide range of affinities for hrPrP, suggesting that RNA structure plays a role in the specificity and stability of the interaction. This invention further characterizes the RNA-binding activities of PrP and examines, in depth, the effect of shsRNAs on inducing resistance of human recombinant PrPSen to PK digestion. Described in the Examples below are the interactions between shsRNAs and hrPrP in vitro under physiological conditions that can lead to PrP aggregation and consequent conversion of PrPSen into PrPRes. In other embodiments, it is demonstrated that RNAs in these nucleoprotein complexes are protected against hydrolysis by ribonuclease A (RNase A). These results suggest potential molecular mechanisms of in vivo PrP conversion and a possible role of small RNAs in prion disease origin and progression.

New theories are emerging supporting a catalytic role for NAs in the key events of prion disease origin and propagation, such as changes in PrP conformation, PrP polymerization (or oligomerization) and aggregation, and the development of PrP resistance to Proteinase K hydrolysis. The inventors herein disclose the ability of an RNA to transform recombinant PrP into a Proteinase K resistant form (PrPRes) in the absence of PrPSc and under physiological conditions (neutral pH, 37° C.). Other in vitro PrPSen to PrPRes conversion protocols at physiological pH have been described, but they are far more complicated requiring, in addition to PrPSc, cyclic sonication steps or the presence of membrane rafts

This work characterizes interactions between PrP proteins from various sources with small, highly structured RNAs (shsRNAs), focusing on the interactions between human recombinant PrP (hrPrP) and the RNA, RQ11+12. RQ11+12 binds to PrPC derived from mouse and hamster, suggesting species cross-reactivity, similar to monoclonal antibodies generated against PrP epitopes common to many mammalian species. Multiple PrP proteins bind to a single RQ11+12 RNA, as demonstrated by the dose dependent shift observed in FIG. 27. This activity is similar to other RNA-binding proteins that bind in excess to a single NA ligand like the NCp7 and Rev proteins from HIV-1. The stoichiometry of binding between PrP molecules and RQ11+12 suggests that several molecules of PrP bind to a single RNA molecule. Our data estimates that approximately 15 molecules of hrPrP can bind to a single molecule of RQ11+12, depending on the initial concentration of the protein.

Although, other RNAs bind to hrPrP, in this study only RQ11+12 efficiently converts hrPrP to a Proteinase K resistant form in the presence of BCS (FIGS. 24 and 28). The acquired resistance of hrPrP to PK hydrolysis is presumably the result of a structural transformation in hrPrP. Interestingly, the maximum level of hrPrP resistance to PK hydrolysis occurs at a PrP to RNA ratio of 15:1, correlating well with the results of the gel shift experiments where the same ratio produced the maximum shift of RQ11+12/hrPrP complexes (FIG. 27). Removal of BCS from the incubation mixture resulted in complete digestion of hrPrP (FIG. 29), indicating that a serum component was involved in the conformational change of hrPrP, demonstrating that a cellular factor (or factors) in addition to PrPSc is involved in disease transmission. The serum component, that we call Q-factor, presumably induces further structural changes to the RNA/PrP complex that are already extensive as demonstrated by the lack of RNA degradation by RNase A when in complex with hrPrP (FIG. 30). Even though not all forms of PrPRes are infectious (PrPSc), It has been well-established that PrP structure transition is part of the cascade of events that lead to prion disease. Therefore, the in vitro PrP-conversion system presented by the invention serves as a rapid and powerful pre-screening system for drugs and/or biologics to treat prion diseases or for components involved in the essential steps of prion disease transmission and progression.

Taken together, these results suggest that additional, Q-factor-dependent interactions occur between hrPrP and RQ11+12 that lead to the formation of PrPRes. Although, RQ11+12 is by far the most efficient RNA in this study at inducing the formation of PrPRes, another RNA also induced some level of protection from PK digestion (SC-2).

This is an important finding because it suggests that RNAs other than RQ11+12, which is an artificial construct, may have the same effect in vivo. It is tempting to suggest that a small, non-coding RNA, a class of nucleic acids that are abundant in the cell (35), may assist PrPC conversion in vivo. This would be consistent with the conclusion of Prusiner's group that if a nucleic acid were involved in disease transmission, it would have to be short, likely smaller than 250 nt. We propose that shsRNAs may play a role as a catalyst in PrP structure transition in vivo.

The present invention reveals potential functional interactions between PrP and nucleic acid (NA) molecules. The accumulated knowledge on PrP/NA interactions has advanced our understanding of prion diseases at the structural level, shed new light on the protein-only and “protein X” hypotheses, and in addition can serve as an essential model in understanding the mechanism of PrPC PrPSc transition cascade, PrPSc formation and propagation at the molecular level. Thus, this disclosures demonstrates that RNAs and DNAs are able to facilitate Proteinase K resistance in PrPC and, the transition to the PrPSc isoform.

### Methods of Using the RNA Ligands of the Present Invention

Methods of the present invention are directed towards detecting the presence or absence of one or more agglomeration proteins within a sample matrix. An affinity complex is formed by contacting the sample matrix with one or more probes of the present invention. The term “contacting” herein refers to a bringing together of two or more items in a manner consistent with forming an admixture. The probes of the present embodiment comprise the amplibodies of the instant invention.

As used herein, “sample matrix” includes any sample that contains proteins. For example, blood, urine, other bodily fluids, cells, cell extract, tissue and tissue extract (especially neurological tissue) are within the scope of this invention.

Amplibody probes of the present method comprise one or more nucleic acid amplibody molecules having affinity for at least one agglomeration protein. In one embodiment, the RNA polynucleotide component is a naturally or non-naturally occurring molecule with twenty or more ribonucleotide bases. At least one nucleotide sequence portion of this RNA molecule has affinity to at least one consensus sequence present in the agglomeration RNA-binding protein. In this embodiment, the portion of RNA having affinity for agglomeration proteins is a sequence that is derived from either an RNA virus, an RNA phage, a mRNA, a rRNA, a tRNA, a sequence that is received as a template by one or more RNA dependent RNA polymerases, or a combination thereof.

The amplibody preparation can be a homogeneous collection of amplibody. This homogeneous preparation is analogous to a monoclonal antibody preparation. Alternatively, the amplibody preparation can be heterogeneous which is analogous to a polyclonal preparation of antibody.

The instant method also includes the use of a heterogeneous collection of amplibodies. This collection refers to different amplibodies having affinity for different binding sites on the same or different agglomeration proteins. A heterogeneous collection of amplibodies can be introduced to a sample matrix. Affinity complexes can form between a single agglomeration protein and different amplibodies binding at different sites along the agglomeration protein. Additionally, affinity complexes can be formed between different amplibodies and different agglomeration proteins present within the one sample matrix.

The present method includes the detection of any affinity complex formed. The detection of an affinity complex is indicative of the presence of one or more agglomeration proteins present in the original-sample matrix. Detection of the affinity-complex can be accomplished by a method selected from the group consisting of mass or density measurement, mass spectrometry, plasmon resonance, optical emission or absorption, fluorescence, phosphorescence, luminescence, chemiluminescence, polarization, refractive index changes, electrical conductivity, radioactivity, viscosity, turbidity, and optical rotation.

One or more of the amplibody probes can be labeled. The label can be bound ionically, covalently, or via adsorption. Preferably, the label is bound covalently to any region of the amplibody polynucleotide comprising the sequence of interest and that does not interfere with binding to an agglomeration protein. The label can include, but is not limited to, radioactive isotopes, such as a radioactive phosphorous atom, affinity reagents, such as biotin, intercalating fluorescent dyes, or a fluroescent moiety attached to the amplibody, phosphorescent dyes or chelates, electrophores for detection by mass spectrometry, chemiluminescent moiety chromophores.

Using a Photodyod Array (PDA) detector, a spectral analysis can be performed without the employment of any label per se. The affinity complex (as well as the amplibody) can be monitored and detected by its unique spectral image using a PDA detector. Separation of free amplibody from complexed amplibody (complexed with agglomeration protein) can be accomplished using methods known to those skilled in the art, for example, employing size-exclusion chromatography, filtration, electrophoresis or alike.

The present invention also pertains to a method for detecting one or more agglomeration proteins within a sample matrix using one or more immobilized amplibody probes. In this embodiment, at least one RNA polynucleotide is immobilized to a solid surface. The immobilized polynucleotide can be an amplibody, alternatively, it can hybridize to a second RNA polynucleotide that is an amplibody. This method also extends to a heterogeneous collection of amplibodies, each amplibody having affinity for a different agglomeration protein, alternatively, each amplibody having affinity for a different site on the same agglomeration protein, or a combination of both.

In one aspect of this embodiment, a first polynucleotide (can be either DNA or RNA) that comprises a sufficient sequence for hybridizng, under suitable conditions, to a second polynucleotide (that is an amplibody) is immobilized to a surface. See U.S. Ser. Nos. 08/971,845; 06/016,708; and 08/812,105; the entire teachings of which are incorporated herein by reference. The portion of the first polynucleotide that is used for immobilization is different from the sequence region that is employed to hybridize to the second polynucleotide (or amplibody). In this aspect of the invention, the method can be performed sequentially or as a single step. First, a sample matrix is admixed with an appropriate amplibody mixture, either homogeneous or heterogeous mixture of amplibodies, under conditions suitable for forming an affinity complex by interacting amplibody and agglomeration protein. Following this step, the putative affinity complex can be admixed with the first polynucleotide that is immobilized to a surface under conditions suitable for hybridization between the first polynucleotide and second polynucleotide. Second, a sample matrix is admixed with a hybridization complex formed from the first polynucleotide hybridized to the second polynucleotide, wherein the first polynucleotide is immobilized to a surface. Under these conditions, affinity complex formation occurs with an immobilized amplibody. Preferably, any free affinity complex will eventually become immobilized by the hybridization of the second polynucleotide (amplibody)-agglomeration complex with the immobilized first polynucleotide.

In another aspect of this embodiment, the immobilized amplibody is immobilized to a surface using a region of the polynucleotide not affecting the amplibody's ability to interact with an agglomeration protein. The aggomeration protein directly forms an affinity complex with the immobilized amplibody.

Means of attaching a nucleic acid to a surface support, such as a solid support surface, can be by simple adsorption. Preferably, the attachment is mediated through a covalent-bond between the nucleic acid and some chemical moiety associated with the support surface, for example, an amine or carboxyl group, or acrylamide. Chemical crosslinkers can be employed to immobilize a nucleic acid to a surface. An example of such a chemical crosslinker is carbodiimide (such as, 1-ethyl-3,3-dimethylaminopropyl-carbodiimide) which can be used to link the phosphate group on the 5′ end of a nucleic acid with an amine group on the surface of a support. Additionally, ionic interactions can also facilitate such immobilization of the nucleic acid. The binding can be direct as between the nucleic acid and surface, or indirect such that an intermediate molecule lies between the nucleic acid and the surface. The intermediate molecule need not have any precise length.

Affinity reagents can also be employed to immobilize nucleic acid to a surface. For example, a nucleic acid carrying avidin or biotin moieties to a surface containing the cognate moiety, will bind the nucleic acid to the surface. Another example of using an affinity-based immobilization technique is to coextensively link the nucleic acid of interest to an affinity ligand, for example, biotin or avidin. The cognate receptor to the ligand, for example, if biotin is the ligand, then avidin will be the cognate receptor, will have attached to it a magnetic particle. When a magnetic field is applied to the surface, the magnetic particle, along with what is attached thereto, will be immobilized to the surface.

The support matrix can be a chromatographic support in the form of beads or particles, dipsticks, fibers, containment vessels, thin-layer plates, membranes, gels like polyacrylamide, starch, agarose, cellulose or other polymeric gels.

A kit for determining the presence or absence of one or more agglomeration proteins within a sample matrix is also disclosed herein. The kit comprises one or more amplibody probes. These probes comprise a non-naturally occurring RNA with twenty or more nucleotides, wherein at least one sequence portion of the nucleotides has affinity for the agglomeration proteins. In one embodiment, the affinity sequence portion of the amplibody is derived from either an RNA virus, an RNA phage, a nucleotide sequence that can be received as a template by one or more RNA dependent RNA polymerases, a mRNA, a rRNA, a tRNA, or a combination thereof. In practice, this kit employs the method of the present invention.

In one aspect, the kit has at least two amplibodies, each binding to a different site on the agglomeration protein. At least one amplibody in this embodiment has a label that can be detected via conventional methods. In a particular aspect, one of the amplibodies of the kit is immobilized to a solid support. Other amplibodies in this particular aspect can have affinity for an agglomeration protein and have the ability to hybridize to the immobilized amplibody. In this particular aspect of the invention, preferably the free amplibody is labeled.

The kit can further comprise a means for separating an affinity complex, comprising one or more amplibodies and at least one agglomeration protein, from free amplibody. The separation means can include, but are not limited to, size-based membranes, filtration units, and alike.

### Prion Protein Diseases

In a preferred embodiment, the NA antibodies of the invention are directed to a prion protein; prion proteins being a protein involved in the disease cascade or progression of Transmissible spongiform encephalopathies (TSEs). TSEs are neurodegenerative infectious diseases that affect the central nervous system (CNS). TSEs include scrapies in sheep, Bovine Spongiform Encephalopathy (BSE) in cattle and Cruetzfeld-Jakob Disease (CJD), Guerstmann-Straussler-Scheinker Syndrome (GSS), kuru, and Fatal Familial Insomnia (FFI) disease in humans (1). Common to all of these fatal diseases are long incubation periods and the accumulation of amyloid-like rods or scrapie associated fibrils (SAFs). The formation of SAFs is the result of extensive fibrillation of PrP^{Sc}, the isoform of the endogenous and innocuous PrP^{C} protein that is associated with infectivity. The structural transformation of the soluble PrP^{C} to the insoluble PrP^{Sc} isoform marks the onset and progression to clinical prion disease.

The gene that encodes PrP^{C} is highly conserved and constitutively expressed from the pmP locus as a 35 kDA glycoprotein (Chesebro et al., 1985 Nature 315:331-33; Oesch et al., 1985 Cell 40:73546; the entire teachings of which are incorporated herein by reference). Approximately one half of translated PrP^{C} is processed to the extracellular membrane where it is anchored to the plasma membrane by a C-terminal glycosyl-phophatidyl-inositol (GPI) anchor. However, PrP^{C} has also be found in two trans-membrane forms, one with the N-terminus inside the ER lumen (PrP^{Ntm}; 40-50%) and the other in the opposite orientation with the C-terminus inside the ER lumen (PrP^{C}tm; 10%). It is unknown if these processing differences reflect the functional properties of these PrP^{C} forms.

PrP proteins have interesting structural characteristics, particularly the extraordinary transformation from its native, wildtype conformation PrP^{C} to the infectious PrP^{Sc}. The alteration in protein structure is marked by a transition from alpha-helix rich of PrP^{C} into beta-sheet rich regions in the C-terminal domain of the isoform associated with infectivity (Pan et al., 1993 PNAS, USA 90:10962-66; the entire teachings of which are incorporated herein by reference). Several biochemical traits distinguish the isoforms such as the insolubility of PrP^{Sc} in physiologic solutions and the resistance of its C-terminal domains (amino acids 90-231) to digestion by proteinase K. The structure of the non-protease treated full-length N-terminus of PrP is very flexible and without a single, stable structure based on NMR structural studies. Therefore, this region of PrP is most likely indistinguishable between the cellular and scrapie isoforms.

The binding of PrP to nucleic acids has been demonstrated many times through the observation of direct complex formation in vitro with purified protein and by copurification of nucleic acids from scrapie associated fibrils (SAFs) removed from infected tissue (Merz et al., 1981 Acta Neuropathol (Berl) 54(1):63-74; the entire teachings of which are incorporated herein by reference). For example, several thousand bases of the viral RNA genome of LAP were co-purified with SAF from infected tissue (Murdoch, et al., 1990 Virology 64(4):1477-86; Akowitz et al., 1994 NAR 22(6): 1101-07; the entire teachings of which are incorporated herein by reference). Such observations influenced studies to explore the possibility that nucleic acids were a required genetic component in the transmission of TSE, although no such genetic link has been experimentally determined to date. Indirect evidence for an in vivo association between PrP and viral components is the observation that the rate of PrP^{Sc} formation is accelerated in cells affected with moloney murine leukemia virus (Carp et al., 1999 J Gen Virol 80(Pt 1):5-10; the entire teachings of which are incorporated herein by reference). There is further evidence of interactions between PrP and viral nucleic acids derived from in vitro studies that used recombinant, mammalian PrP proteins expressed in E. coli. Syrian Golden Hamster recombinant PrP^{C} (srPrP) has a surprising homology of in vitro activities with the nucleocapsid protein from HIV (Ncp7)(Tanchou et al., 1995 J Mol Biol 252:563-71; Gabus et al., 2001a J Mol Biol 307(4):1011-21; Gabus et al., 2001b J Biol Chem 276(22):19301-9; the entire teachings of which are incorporated herein by reference). srPrP has virtually the same level of activity as Ncp7 in the processes of DNA strand-transfer, nucleic acid chaperoning, HIV-RT priming, and the formation of condensed protein/nucleic acid structures. Double stranded DNA also induced the formation of similar condensed PrP structures, as well as resistance to proteinase K digestion (Nandi, 1998 Arch Virol 143(7):1251-63; Nandi and Leclerc, 1999 Arch Virol 144(9):1751-63; Nandi and Sizaret, 2001 Arch Virol 146:32745; the entire teachings of which are incorporated herein by reference). Recently, two small RNA aptamers have been isolated based on their ability to bind to PrP proteins. One aptamer, AP1 (29 nt), was isolated using recombinant srPrP, and is predicted to fold into a compact structure containing three stacked G-quartets, a structure suggested to be important in binding to srPrP (FIG. 12a; Weiss et al., 1997 J Virol 71(11):8790-97; the entire teachings of which are incorporated herein by reference). Using a series of srPrP truncations, the authors localized the binding of AP1 to the flexible N-terminus, within amino acids 23-39.

The specific mechanisms of initiation and progression of prion fibrillation are not well understood. Previous studies have tried to determine if RNA or DNA play a genetic role in the transmission of prion disease (Akowitz et. al. 1994; Nandi and Leclerc, 1999; Cordeiro et. al., 2001 J Biol Chem 276(52):49400-09; Narang 1998 Res Virol 149(6):375-82; Narang 2002 Exp Biol Med 227(1):4-19; the entire teachings of which are incorporated herein by reference).

The features and other details of the invention will now be more particularly described and pointed out in the examples. It will be understood that the particular embodiments of the invention are shown by way of illustration and not as limitations of the invention. The principle features of this invention can be employed in various embodiments without departing from the spirit and scope of the invention.

### Other Peptides and Embodiments

In other embodiments, the oligonucleotides of the invention can include other appended groups such as peptides, e.g., for targeting host cell receptors in vivo, or agents facilitating transport across the cell membrane (see, e.g., Letsinger et al. (1989) Proc. Natl. Acad. Sci. USA 86:6553-6556; Lemaitre et al. (1987) Proc. Natl. Acad. Sci. USA 84:648-652; PCT Publication No. WO88/09810) or the blood-brain barrier (see, e.g., PCT Publication No. WO 89/10134). In addition, oligonucleotides can be modified with hybridization-triggered cleavage agents (see, Krol et al. (1988) Bio-Techniques 6:958-976) or intercalating agents (see, Zon (1988) Pharm. Res. 5:539-549). To this end, the oligonucleotide may be conjugated to another molecule, e.g., a peptide, hybridization triggered cross-linking agent, transport agent, or hybridization-triggered cleavage agent. Finally, the oligonucleotide may be detectably labeled, either such that the label is detected by the addition of another reagent, e.g., a substrate for an enzymatic label, or is detectable immediately upon hybridization of the nucleotide, e.g., a radioactive label or a fluorescent label, e.g., a molecular beacon as described in U.S. Pat. No. 5,876,930.

Another aspect of the invention pertains to vectors, preferably expression vectors, containing a nucleic acid encoding a marker protein of the invention (or a portion thereof). As used herein, the term “vector” includes a nucleic acid molecule capable of transporting another nucleic acid to which it has been linked. One type of vector is a “plasmid”, which includes a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced, e.g., bacterial vectors having a bacterial origin of replication and episomal mammalian vectors. Other vectors, e.g., non-episomal mammalian vectors, are integrated into the genome of a host cell upon introduction into the host cell, and thereby are replicated along with the host genome. Moreover, certain vectors are capable of directing the expression of genes to which they are operatively linked. Such vectors are referred to herein as “expression vectors.” In general, expression vectors of utility in recombinant DNA techniques are often in the form of plasmids. In the present specification, “plasmid” and “vector” can be used interchangeably as the plasmid is the most commonly used form of vector.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, which is operatively linked to the nucleic acid sequence to be expressed. Within a recombinant expression vector, “operatively linked” is intended to mean that the nucleotide sequence of interest is linked to the regulatory sequence(s) in a manner which allows for expression of the nucleotide sequence, e.g., in an in-vitro transcription/translation system or in a host cell when the vector is introduced into the host cell The term “regulatory sequence” is intended to include promoters, enhancers and other expression control elements, e.g., polyadenylation signals. Such regulatory sequences are described, for example, in Goeddel; Gene Expression Technology: Methods in Enzymology 185, Academic Press, San Diego, Calif. (1990). Regulatory sequences include those which direct constitutive expression of a nucleotide sequence in many types of host cells and those which direct expression of the nucleotide sequence only in certain host cells, e.g., tissue-specific regulatory sequences. It will be appreciated by those skilled in the art that the design of the expression vector can depend on such factors as the choice of the host cell to be transformed, the level of expression of protein desired, and the like. The expression vectors of the invention can be introduced into host cells to thereby produce proteins or peptides, including fusion proteins or peptides, encoded by nucleic acids as described herein, e.g., marker proteins, mutant forms of marker proteins, fusion proteins, and the like.

The recombinant expression vectors of the invention can be designed for expression of marker proteins in prokaryotic or eukaryotic cells. For example, proteins can be expressed in bacterial cells such as E. coli, insect cells (using baculovirus expression vectors) yeast cells or mammalian cells. Suitable host cells are discussed further in Goeddel, Gene Expression Technology: Methods in Enzymology 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith, D. B. and Johnson, K. S. (1988) Gene 67:31-40), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein.

Purified fusion proteins can be utilized in marker activity assays, e.g., direct assays or competitive assays described in detail below, or to generate antibodies specific for marker proteins for example.

Examples of suitable inducible non-fusion E. coli expression vectors include pTrc (Amann et al., (1988) Gene 69:301-315) and pET 1 d (Studier et al., Gene Expression Technology: Methods in Enzymology 185, Academic Press, San Diego, Calif. (1990) 60-89). Target gene expression from the pTrc vector relies on host RNA polymerase transcription from a hybrid trp-lac fusion promoter. Target gene expression from the pET 11d vector relies on transcription from a T7 gn10-lac fusion promoter mediated by a coexpressed viral RNA polymerase (T7 gn1). This viral polymerase is supplied by host strains BL21(DE3) or HS174(DE3) from a resident prophage harboring a T7 gn1 gene under the transcriptional control of the lacUV 5 promoter.

One strategy to maximize recombinant protein expression in E. coli is to express the protein in a host bacteria with an impaired capacity to proteolytically cleave the recombinant protein (Gottesman, S., Gene Expression Technology: Methods in Enzymology 185, Academic Press, San Diego, Calif. (1990) 119-128). Another strategy is to alter the nucleic acid sequence of the nucleic acid to be inserted into an expression vector so that the individual codons for each amino acid are those preferentially utilized in E. coli (Wada et al., (1992) Nucleic Acids Res. 20:2111-2118). Such alteration of nucleic acid sequences of the invention can be carried out by standard DNA synthesis techniques.

Another aspect of the invention pertains to host cells into which a nucleic acid molecule of the invention is introduced within a recombinant expression vector or a nucleic acid molecule of the invention containing sequences which allow it to homologously recombine into a specific site of the host cell's genome. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. Preferably, the host cell is a prokaryotic cell. For example, the invention can be expressed in bacterial cells such as E. coli. Other suitable host cells are known to those skilled in the art.

Vector DNA can be introduced into host cells via conventional transformation or transfection techniques. As used herein, the terms “transformation” and “transfection” are intended to refer to a variety of art-recognized techniques for introducing foreign nucleic acid, e.g., DNA, into a host cell, including calcium phosphate or calcium chloride co-precipitation, DEAE-dextran-mediated transfection, lipofection, or electroporation Suitable methods for transforming or transfecting host cells can be found in Sambrook, et al. (Molecular Cloning: A Laboratory Manual. 2nd, ed., Cold Spring Harbor Laboratory, Cold Spring Harbor Laboratory Press, Cold Spring Harbor, N.Y., 1989), and other laboratory manuals.

A host cell of the invention, such as a host cell in culture, can be used to produce, i.e., express, a recombinant protein. Accordingly, the invention further provides methods for producing a protein using the host cells of the invention. In one embodiment, the method comprises culturing the host cell of invention (into which a recombinant expression vector encoding a protein, or proteins, has been introduced) in a suitable medium such that a protein of the invention is produced. In another embodiment, the method further comprises isolating a protein from the medium or the host cell.

Of course, one skilled in the art will appreciate further features and advantages of the invention based on the above-described embodiments. Accordingly, the invention is not to be limited by what has been particularly shown and described, except as indicated by the appended claims.

### Example 1

An example of a protocol for forming an agglomeration complex is as follows: Incubate about 0.83 pmoles of human recombinant prion protein (“hrPrP”, a prion protein) with about 0.2 pmoles (or with 1.2 pmoles) of a NA antibody like RQ11+12. The RNA-protein binding is performed in 20 μl of a reaction mixture consisting of approximately 50 mM MOPS, pH 7.4; 5 mM MgCl₂; 50 mM LiCI; 1 mM DTT; 1 μg tRNA; 80-100 μg/μl BCS; 0.05% DOXY and 0.05% NP-40. The reaction mixture is incubated for about 20 minutes at around room temperature.

The prion protein employed can be hrPrP, PrP^{C} as well as other prion proteins. The proteins will form one or more agglomerations under suitable conditions. These agglomerations can then be detected, for example, using electron microscopy. The formation of an agglomeration complex is indicative of the presence of a CBF in the reaction mixture.

### Example 2

The method of Example 1 can be augmented by examining protease K resistance of the prion protein. In the absence of a NA antibody and/or CBF, prion proteins are soluble and readily digested by protease K. Thus, a different detection mechanism can be employed for the method of Example 1:

Following the incubation of sample matrix with NA antibody and prion protein (PrP), proteinase K is added to the admixture. The complex formation is performed as outlined in Example 1. Resistance of PrP to protein digestion is analyzed by treating the RNA-PrP-CBF complex with protease K. For example, to the admixture, approximately 2 μg of protease K (available from commercial sources) is added to the reaction mixture and incubated for about 60 minutes at around 37° C. The reaction can be terminated using approximately 20 μL of 2× sample buffer (available from NOVEX) containing 10 mM of phosphormethylsulphonylfluoride (PMSF). Aliquots of the reaction can then be analyzed by SDS/PAGE analysis. Samples are loaded in a 6% polyacrylamide gel with 7 M urea. Electrophoresis is performed for about 60-90 minutes at around room temperature using a 1×TBE buffer (50 mM Tris-borate, pH 8.3, 0.5% DOXY, 0.5% NP-40 and 1 mM EDTA). The gel is subsequently dried and exposed to x-ray film or analyzed by phosphoimager (Bio-Rad, BIL-20). Those practitioners skilled in the art are very familiar with gel electrophoresis and alike analysis. If a Western blot is prepared, then the resistant PrP can be visualized using immuno-blot technique using, for example, monoclonal antibody (MoAb) 3F-4 that is specific for the amino acid 109-112 epitope of hamster PrP and 7A-12 MoAb specific for the amino acid 100-145 epitope of mouse PrP.

### Example 3

The following procedure has been employed by the inventors to construct a fraction of human serum enriched with a CBF:

An aliquot, e.g., 5 mL, of human plasma (no hemolysis) was mixed with 1 mL of polyethylene glycol (PEG). The mixture was allowed to stand at room temperature for approximately 15 minutes. The mixture was then centrifuged at approximately 3500×g for about 15 minutes. The supernatant was then decanted. The pellet was gently washed using approximately 2 mL of saline, then resuspended in approximately 5 mL of saline-EDTA using gentle sonication. The suspension was gently washed with approximately 2 mL saline and allowed to stand for about 2 minutes, the supernatant was then removed. The remaining pellet was resuspended with about 1 mL of saline using sonication. The suspension was dialyzed in order to remove PEG and stored at 4° C.

Next, the precipitated complex was incubated in solution of 1% SDS, 50 mM DTT, 50 mM TRIS-HCl, pH 6.7, 20% glycerol for approximately 10 minutes at around 95° C. Electrophoresis was then performed with a consequent extraction of the appropriate protein fraction, or the sample was applied onto a fractionation column and the sample eluted with 1% SDS, 50 mM DTT, 50 mM TRIS-HCl (pH 6.7), and 20% Glycerol.

### Example 4

As example of an analysis to determine the efficacy of an agent in inhibiting formation of an agglomeration complex:

A prion protein (e.g., hrPrP) is obtained and labeled using, for example, fluorescent dye Cy5 (purchased from Amersham). (Typically, the labeling protocol is provided by the vendor.) Approximately 1.6 pmoles of NA antibody like RQ11+12 RNA is incubated in a binding buffer (approximately 50 mM MOPS, pH 7.5; 1 mM EGTA; 50 mM LiCI; 5 rnM MgCl₂; 1 mM DTT; 0.05% NP-40; 0.05% DOX; 5% glycerol; 1 μg BSA; 1 μg tRNA) with about 0.3 nmol of prion protein together with bovine calf serum, (about 80 μg protein) in about 10 μL of the binding buffer in the presence of a pharmaceutical test agent for about 1 hour at around ambient temperature. Then about 0.5 μg protease K is added and the mixture incubated for about 30 min. at about 37° C. The digestion reaction can be stopped by the addition of about 5 mM PMSF and about 12 μL of 2×SDS protein sample buffer. Then the reaction sample is boiled for about 10 min. at around 95° C. Samples can then be subjected to electrophoreses in PAAG (4-20%, available from NOVEX).

### Example 5

### Protease K Protection

Seven pmoles of hrPrP was incubated with 123 fmoles of the RQ11+12 RNA (“RQ RNA”) in 10 μL binding buffer for 17 hours at room temperature. Next, the protease K (50 ng/μL) was added and the sample was incubated for 30 minutes at 37° C. The proteolysis was terminated by the addition of PMSF (5 mM) and 10 μL of 2×SDS sample buffer. The sample was then incubated for 7 minutes at 95° C. and analyzed by PAGE and immunoblotted using 3F-4 anti PrP antibody (available from Q-RNA).

Western immunoblotting data using anti-PrP monoclonal antibody (3F-4) illustrates that hrPrP can be protected from protease K digestion if the CBF is present in the RQ RNA-PrP admixture during complex formation. The CBF was in fact present in the bovine calf serum (“BCS”).

### Example 6

### Presence of CBF in Human Serum

FIG. 45 depicts two gels demonstrating the presence of a CBF in human serum. RQ RNA was incubated in 10 μL of a binding buffer (50 mM MOPS, pH 7.5; 1 mM EGTA; 50 mM LiCI; 5 mM MgCl₂; 1 mM DTT; 0.05% NP-40; 0.05% DOX; 5% glycerol; 1 μg BSA; and 1 μg tRNA) with 250 ng of hrPrP in the presence of human or bovine calf serum (80 μg protein) for 1 hour at room temperature. Then 0.5 μg protease K was added and incubated for 30 minutes at 37° C. The digestion was arrested by the addition of PMSF (5 mM) and 12 μL of 2×SDS protein sample buffer. Samples were then heated for 10 minutes at 95° C. Samples were then run in PAAG (4-20%, NOVEX) and immunoblotted using 3F-4 monoclonal antibody to hrPrP. Western immunoblotting with anti-PrP monoclonal antibody (3F-4) data demonstrated that different preparations of human serum have a unique ability to generate the resistance of hrPrP to protease K digestion indicating the presence of CBF in these samples.

### Example 7

### PrP Protection Assay

Human recombinant prion (hrPrP), refolded from Guanidine-HCl-SDS solution, was used for labeling with fluorescent dye Cy5 (purchased from Amersham). (Labeling protocol is provided by the vendor.) Approximately 1.6 pmoles of RQ11+12 RNA was incubated in a binding buffer (50 mM MOPS, pH 7.5; 1 mM EGTA; 50 mM LiCI; 5 mM MgCl₂; 1 mM DTT; 0.05% NP-40; 0.05% DOX; 5% glycerol; 1 μg BSA; 1 μg tRNA) with 0.3 nmol of hrPrP together with bovine calf serum, (80 μg protein) in 10 μL of the binding buffer in the presence of chlorpromazine for 1 hour at ambient temperature. Then 0.5 μg protease K were added and the mixture was incubated for 30 min. at 37° C. The digestion reaction was arrested by the addition of 5 mM PMSF and 12 μl of 2×SDS protein sample buffer. Then the reaction sample was boiled for 10 min. at 95° C. Samples were then subjected electrophoreses in PAAG (4-20%, NOVEX).

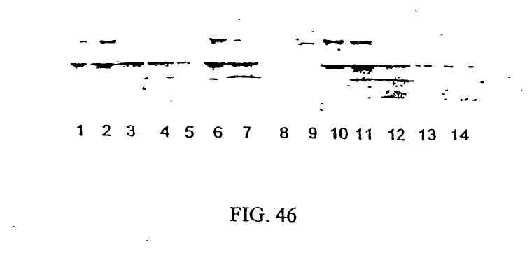

FIG. 46 depicts the efficacy of chlorpromazine in blocking the PrP protection by serum or lipoproteins in a dose-dependent manner. Specifically, lanes 1, 6, and 10 represent reactions in which there was no protease treatment; lanes 2, 3, 4, 5, 7, 8, 9, 11, 12, 13, and 14, represent protease K treated samples; lanes 3, 7, 12 represent samples having the addition of 20 mM of chlorpromazine; lanes 4, 8, 13 represent samples having the addition of 40 mM chlorpromazine; lanes 5, 9, and 14 represent reactions in which 80 mM chlorpromazine was added. Finally, lanes: 1-5, represent the HS fraction of blood serum; 6-9, represent the LP1 fraction of blood serum; 10-14, represent the LP2 fraction of blood serum. It can be observed that in the presence of chlorpromazine, prion protein's sensitivity to protease K digestion is preserved.

All of the reagents mentioned herein can be obtained from commercial sources or through Q-RNA of New York, N.Y.

While this invention has been particularly shown and described with reference to embodiments thereof, it will be appreciated by those skilled in the art that various changes in form and detail may be made therein without departing from the spirit and scope of the invention as defined by the appended claims hereto.

Animal Models

### Example 2

The desired system for expression of RNA chaperones will contain a selectable marker for use in producing a transgenic cell line. Because this construct will be used to generate transgenic mice in the Phase II project, we chose to use commercially available pTRE-tight vector with tetracycline (tet) inducible promoter system, which, according to Clontech specification, has a lower background of expression than the previously available pTRE vector and is known to work in transgenic mice. The tet system uses the tet derivative doxycycline as an inducer of tightly regulated in vivo in a dose-dependent manner over a wide range of doxycycline concentrations and requires administration of doxycycline only when transgene expression is required. Doxycycline is a safe and effective clinical and veterinary antibiotic that is inexpensive and widely available.

### Work in Progress: Modifications have been made to the Original pTRE Tight Vector

In order to ensure the functions of the newly synthesized RNA chaperone in mammalian cell culture and live animals, its original nucleotide composition must be preserved or, changed minimally if it is unavoidable. This can be achieved by minimizing a number of additional ribonucleotides to the 5′ and 3′ ends of the newly transcribed RNA. For this, we designed three pairs of oligonucleotides, Oligo I for the truncated 3′ end of minimal CMV promoter, Oligo II for the modified multiple cloning site, and Oligo III for synthetic polyA signal. Introducing these oligos into a properly digested pTRE vector, we shortened the 3′ end of the pol II CMV promoter to allow close juxtaposition of the RNA coding sequence to the transcription start site. A CMV was chosen in order to obtain sufficient accumulation of the shsRNA in the cytoplasm. Finally, to make the system functional, the mCMV promoter was put under the control of the upstream tetracycline response element (TRE). Expression of the RNA chaperone, therefore, will depend upon the presence of the tetracycline operator regulatory protein (tet R).

We also modified the multiple cloning site (mcs) of pTRE, leaving only two cloning sites, BamHI and HindIII, which we are planning to use to clone the RNA chaperone template. As a result of this, six extra ribonucleotides only will be added to the 5′ end of the RNA during its synthesis. Finally, we substituted original SV40, 200 bp long, poly A signal with a shorter, minimal 60 bp version that efficiently terminated the transcripts and introduced a short polyA tails at the 3′end of the transcribed RNA. The correct nucleotide composition of this modified pTRE-tight vector was verified by restriction site mapping, PCR with test-primers and by sequencing of both DNA strands through the entire modified region and across the junctions with the vector backbone.

FIG. 11 represents a map of the vector for RNA chaperone cloning and expression in mammalian cell culture and live animals.

Experimental Strategy.

The tet induction system requires two constructs: the inducible (response) vector, in our case pRevTRE-tight modified or pAdenoTRE tight (modified) that will code for the RNA chaperone, and, the regulator vectors, pRev tet on or pAdeno tet on, that express the reverse tet transactivator (rtTA) under the control of the CMV promoter (both vectors from Clontech). To generate stable lines in PC12 cells the strong CMV promoter will be used to drive expression of rtTA. On the other hand, for primary neuronal cell lines expressing the RNA chaperone, the CMV promoter will be replaced by the neuron specific promoters, neuron specific enolase (NSE) or Ca, calmodulin dependent kinaseIIα(CamkIIα). The reason being that commercially available neuronal primary cultures contain glial cells, so transgene expression needs to be specifically targeted to the neuronal population in the cultures. Both promoters were tested, and the more efficient one will be used in subsequent experiments in vitro as well as in the generation of transgenic mice

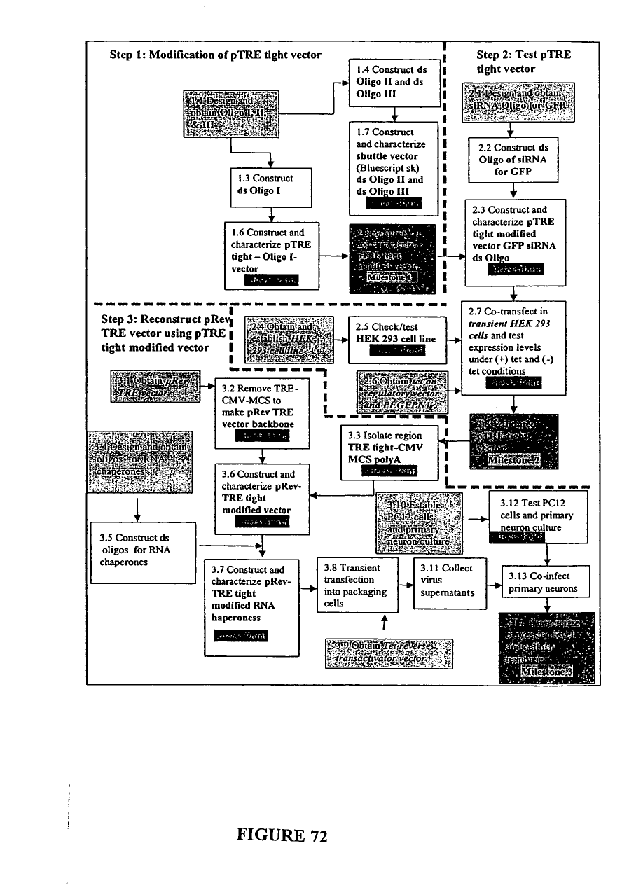

Generation of Neuronal Cell Lines Expressing Exogenous Chaperone RNA (FIG. 72)

To generate the neuronal cell lines that conditionally express the RNA chaperone two types of neuronal cultures will be used. One is the established rat neuronal cell line PC12 (obtained from ATCC) and the other rat or mouse primary neuronal cultures (PNC) (obtained from Cambrex).

The PC12 neuronal cell line was used in these experiments because in spite of being an established line, it has retained most of the functional characteristics of neurons. This line is widely used as a neuronal cell model in many fields of studies. The fact that it is an established line will make easier the generation of stable lines expressing chaperone RNAs because a rigorous selection of clones with high inducibility can be achieved. PNC, on the other hand, have a finite lifespan. This factor needs to be taken into account when selecting the delivery system for the chaperone RNA. The advantage of using PNC is that they are the closest in vitro counter part of the neurons in the brain. PNC often contains glia cells, which affect neuronal function and survival in vivo.

The flow chart (FIG. 72)(including Step 1 that was accomplished as a “work in progress”) represents the overall construction process for tetracycline induction system that regulates RNA transgene expression in cells and transgenic animals. The entire program consists of three Parts or Steps. Each Step of this project consists of the several individual tasks with well-defined objectives, such as construction, modification, or obtaining commercially available constructs or cell lines. The success of each part of the Phase I work were judged on the success of the tasks with appropriate check points, in which nucleotide composition of the constructed vectors and their components will be validated by electrophoretic mobilities, restriction mapping and/or sequencing.

Step 1 represents a preparative part of the project, in which a commercially available pTRE-tight vector was modified for our specific needs. In Step 2 we evaluated modified pTRE-tight vector to inducible produce RNA, using a model system that consist of GFP protein and appropriate siRNA that can inactivate GFP mRNA. In the final Step 3 constructed inducible vector that can produce RNA chaperones and will use these vectors to generate transgenic murine cell lines. Success of of this project will be judged by the ability of transgenic cell line to inducibly express RNA chaperones and the ability of the RNA chaperones to induce amyloid aggregate formation.

The validated end-product of Step 1, a modified pTRE-tight vector (Milestone 1), was used as a starting point for Step 2, in which this modified vector will be used to clone siRNAoligo and then tested for ability to inducibly generate functional siRNA. When functionality of the pTRE-tight vector is confirmed, the pRev TRE-tight (modified) and pAdeno TRE tight modified vectors will be built using the modifications made to pTRE-tight (modified). The pRevTRE and pAdeno TRE-tight (modified) vectors will be validated by restriction mapping and sequencing. Both vectors will be used to clone oligos for RNA chaperones (Step 3), and these constructs will be characterized by restriction mapping and sequencing. Virus stocks will be produced from pRevTRE and pAdeno TRE-tight (modified) RNA chaperone oligos and also the tet reverse transactivator vector (Step 3), and these retrovirus stocks will be used to co-infect established (PC12 cells) and primary neuronal cultures (Step3). (Note: Steps 3.1, 3.2, 3.6 and 3.7 also include work with pAdenoTRE vector. Step 3.13 includes work with P12 cells)

Validate Functionality of the Constructed pTRE Tight (Modified) Vector by Testing its Efficiency to Drive the Inducible Expression of two Control siRNAs, one for the Green Fluorescence Protein (GFP) and the Other for Luciferase.

We tested the vector generated in Step 1 for its ability for inducible production of RNA. For this, we cloned into it a sequence encoding green fluorescent protein (GFP) siRNA to determine if it diminishes the overall fluorescence in cells expressing GFP constitutively from pEGFPN1 (a GFP expression vector). We transiently co-transfected these two vectors at different ratios into a stable HEK293 cell line that expresses the ptet on vector coding for the rtetTA (cell line obtained from Clontech, cat#630 903). We used the calcium phosphate precipitate transfection method which is known to give high transfection efficiency in HEK293cells. The optimal ratios of the response vector, pTRE-tight (modified) (driving GFP siRNA expression) and the pEGFPN1 vector has to be empirically determined. To establish a proper ratio, we first tested a broad range of 1:1, 10:1 and 20:1 ratios of response vector to pEGFPN1. Transfections were performed in 60 mm tissue culture dishes following a standard protocol described by Clontech. Four plates were transfected with each plasmid DNA ratio. We maintained two plates in the absence and the other two in the presence of Dox. Cultures will be induced for 48-72 hours at which point the level of green fluorescence will be assessed. Subsequently the cultures were processed for RNA isolation by a method that enriches for small RNA isolation. These RNA preparations will be analyzed for the presence of GFPsiRNA by dot or Northern blots or by RNAse protection. The specificity of GFPsiRNA action will be compared to an unrelated siRNA negative control such as beta-galactosidase siRNA in the same vector. Basal expression of the cloned insert will be ascertained in the cultures maintained in the absence of doxycycline.

Construct and Characterize the Retrovirus Vector pRev-TRE tight (modified) to be used to Express RNA Chaperones in Cultured Neurons.

Preparation of pRevTRE-RNA Chaperone, a Retroviral Vector.

Primary neuronal cells are difficult to transfect but are sensitive to another gene delivery system, retrovirus infection. Modifications to pRevTRE are similar to those made in pTRE-tight, except for an extra step, the TRE operator in pRev TRE has to be replaced by the TRE-tight operator within pTRE-tight which will give a lower background expression. We found an easy way to do this modification in a single step as follows. In pTRE-tight (modified), all the regions required for transcription, including the pTRE-tight operator located upstream of the mCMV promoter are flanked by two XhoI sites. This XhoI fragment will be isolated and cloned into pRevTRE using XhoI and Sal I (the latter located in the polylinker region, this site has compatible ends with XhoI and after ligation the XhoI is regenerated). Clones will be characterized by restriction enzyme analysis and sequencing. After this, the vector is ready to clone RQ11+12 or any other chaperone RNA coding sequence using the BamHI and HindIII cloning sites. The siRNA oligo control for GFP previously mentioned will be cloned also to test for vector functionality as described earlier for the pTRE-tight (modified) vector. To clone sequences coding for either siRNA or highly structured RNA, like the one coding for RQ11+12 and other chaperone RNAs, the ideal E. coli strain to use is GT116. By mutating a hairpin specific nuclease in this strain, cloning of hairpin rich sequences should be highly facilitated in these cells. Cells can be obtained from InvivoGen Cat.No. gt-116-21. The Sure cells from Stratagene were useful to clone the siRNA coding oligo. They will also be used to clone sequences coding for chaperone RNAs. To elute DNA fragments from agarose gels, the QIAquick gel extraction kit from QIAGEN will be used (Cat.No.28704).

Preparation of pAdeno TRE Modified-RNA Chaperone an Adenovirus Vector

The linear pAdeno X vector backbone generated by double digestion with I-Ceu I and PI-sceI will be obtained from Clontech (AdenoX system1 (Catalog #630 903). The linear vector will be blunt ended and ligated to the blunt ended XhoI fragment from pTRE tight modified that contained the tet regulated expression cassette (-TRE-minCMV-mcs-min polyA signal-), the same fragment previously used to construct pRev TRE tight modified, except for the blunt ending step. The pAdeno TRE tight modified clones generated will be isolated and characterized by restriction mapping and sequencing. Subsequently, chaperone RNA coding sequences will be cloned into this vector and again, clones will be characterized by restriction mapping and sequencing.

Preparation of Virus Stocks

I) Retrovirus Stock Preparation from pRevTRE-Tight Containing RNA Chaperone Coding Sequence

Retrovirus preparation will be performed following the protocol from Clontech. In brief each of the vectors will be transfected into the packaging line GP-293, two to three days later the medium containing the retrovirus particles will be collected, filtered, titered and stored at −70° C. until used.

II) Adenovirus Stock Preparation from pAdenoTRE-Tight (Modified) Containing RNA Chaperone Coding Sequence

Infectious adenovirus is produced by transfection of HEK293 cells using any standard method. Calcium phosphate DNA precipitate works well.

After transfection cultures are monitored every day until a cytopathic effect becomes apparent (cells start to round up and a fraction of them will detach from the tissue culture dishes). One week later cells are collected and lyse by three freeze-thaw cycles. Cellular debris is removed by centrifugation. Store at −20 until used. Virus titer will be determined by a standard protocol describe by Clontech.

Retrovirus Stock Preparation from pRevTRE-Tight RQ11+12.

Primary neuronal cells are refractory to transfection, but are sensitive to another gene delivery system, retrovirus infection. Retrovirus stocks will be prepared from pRevTRE-tight RQ11+12, from the tet regulatory vector coding for the rtTA, and pRevTRE-tight coding for the siRNA controls described earlier. Retrovirus preparation will be performed following the protocol from Clontech. In brief, each of the vectors will be transfected into the packaging line GP-293, and two to three days later the medium containing the retrovirus particles will be collected, filtered, titered and stored at −70° C. until used.

Criteria to Progress to the Next Specific Aim

In order to progress from Specific Aim I to Specific Aim 2, we will need to characterize and confirm functionality of two vectors, the pRevTRE-tight RQ11+12, from the tet regulatory vector coding for the rtTA and pRevTRE-tight coding for the siRNA controls retrovirus vector. The conformation of their functionality will assure success of their transfection into the packaging cell line GP-293, and collection of retrovirus particles that will be ready for use in Specific Aim 2, after being filtered, titered and stored at −70° C.

If we do not obtain the desired results, we will evaluate an alternative induction system. In the Stratagene ecdysone system, the transactivation factor is composed of two proteins that spontaneously dimerize—the retinoid X receptor (RXR) and the VgEcR fusion protein. Both proteins are produced from a single construct and driven by the same promoter, in our case, beta casein. An internal ribosomal re-entry site (IRES) located between the coding regions of the two genes permits transcription of two proteins from the same promoter. This simplifies expression of the components required in situ for ecdysone induction. In this case, we would prepare Constructs 1 and 2 as described above with elements of the ecdysone system. The ecdysone system [39] is induced with insect and plant ecdysteroids, such as ponasterone A or muristerone A, which are neither toxic nor pharmacologically active in mammals.

We will produce rat or mouse neuronal cell lines that express high levels of RNA chaperones to use in developing a cellular assay to further characterize compounds that are positive in the biochemical assay. Endogenous insoluble protein aggregates will be measured with amyloid-specific dye Congo Red (CR) and fluorometric Thioflavin T (ThT) assays, preferably in a 96-well plate format. Immunocytochemistry will be used to test the co-localization of endogenous amyloidogenic proteins tau, Aβ and α-synuclein with the aggregates. The formation of soluble oligomers will be assayed by dot blot of soluble cell fractions using oligomer-specific antibody pAb A11 (Invitrogen, Kayed 2003). To determine the protein composition of oligomers immunoprecipitation of specific endogenous amyloidogenic proteins will be followed by dot blot analysis with the oligomer-specific antibody.

Co-Infect Established and Primary Neuronal Cultures with Either the Retrovirus Vector, pRev TRE Tight (Modified) or the Adenovirus Vector, pAdeno TRE (Modified), both Coding for RNA Chaperones, Together with the Respective Virus from ptet on Vector that Expresses the Transactivator rtTA.

To generate the neuronal cell lines that conditionally express the shsRNA chaperone, two types of neuronal cultures will be used. One is the established rat neuronal cell line PC12 (obtained from ATCC) and the other rat or mouse primary neuronal cultures (obtained from Cambrex). The PC12 neuronal cell line will be used in these experiments because in spite of being an immortalized line, it has retained most of the functional characteristics of neurons. This line is widely used as a neuronal cell model in many fields of studies and will facilitate generation of stable lines expressing chaperone RNAs because a rigorous selection of clones with high inducibility can be achieved.

Primary neuronal cultures have a finite lifespan in vitro whic needs to accounted for when infecting the cultures. However, the advantage in using primary cultures is that they are the closest in vitro counter part of the neurons in the brain, these cultures often contain glial cells which in vivo affect neuronal function and survival. To restrict expression of the chaperone RNA to the neurons in the culture the rtTA in the regulator vector will be placed under the control of a neuron specific promoter (NSE or Cam kII

For PC12, cultures will be sequentially infected with the regulator and response vector. Cells will be infected first with the tet regulator vector (pRev tet on) which confers neomycin (neo) resistance. A panel of individual stable G418 resistant clones will be isolated, expanded and tested for tight and high doxycycline inducibility by infecting them with the reporter vector pRev TRE luciferase, transiently. Levels of luciferace in the presence and absence of doxycycline will be determined for each of the clones and those with the highest fold induction of luciferase will be selected for infection with retrovirus made from the pRevTREtight (modified) vector coding for the RNA chaperone. This vector confers hygromycin resistance; therefore, a panel of stable clones will be selected in the presence of hygromycin. These double infected clones will be characterized for the inducible expression of the RNA chaperone by northern blot and if necessary, by RT-PCR, performed on RNA preparations from cultures maintained in the presence and absence of doxycycline. Clones with the highest level of induction of chaperone RNA will be further analyzed for the presence of amyloid aggregates.

An adenovirus ‘tet on’ system will be used to infect primary neuronal cultures of mouse or rat origin. Adenovirus vectors do not integrate into the genome, they remain in the infected cells as episomes, therefore stable adenovirus infected cell lines can not be generated. However, the infection efficiency with adenovirus vectors is high, often infected cells receive several copies of the vector. This will allow elevated expression of the transgene in the infected cultures for as long as 2-3 weeks in rapidly dividing cultures and longer if the rate of cell division is low (it could be as long as two to four months). Another advantage of adenovirus is that it can infect dividing and non-dividing cells (for example differentiated neurons) as opposed to retrovirus vectors, that infect only mitotically active cells in culture. This will increase the number and type of cells that can be analyzed for the effect of transgene expression. These reasons suggest that the use of the adenovirus vector, pAdeno TRE tight (modified) expressing chaperone RNAs may be a good approach to test the effect of these RNA molecules in primary neurons. In addition, the establihed PC12 neuronal cell line can be induced to differentiate in the presence of nerve growth factor. These post-mitotic neuronal populations will be easily infected with adenovirus, allowing us to test the effect of elevated chaperone RNA levels in a different cellular background that closely resembles the condition of the post-natal and adult brain where most neurons are post-mitotic. For adenovirus infection the cultured neurons will be plated on poly-D-lysine and laminin coated glass coverslips in 24 well plates. Infections will be performed 12 hours later at a multiplicity of infection (MOI) previously determined. This will be accomplished using a pAdeno β-galactosidase vector and assaying transduction efficiency with a colorimetric substrate. The optimal ratio of regulator to response vector will be also determined with the β-galactosidase vector.

The optimal MOI is the one giving the maximum transduction efficiency with no cytotoxic effects. When infecting with pAdenoTRE tight (modified)chaperone RNA, together with the regulator (pAdeno tet on), parallel cultures will be maintained in the presence and absense of doxycycline to monitor the effect of transgene induction.

Quantify RNA Chaperone Expression Levels in the Generated Cell Lines and Transiently Infected Primary Cultures.

Transfections in this proposal will be performed using the calcium phosphate precipitate technique, which works well for PC12 cells. Retrovirus and adenovirus infection will be used to deliver the transgene into established and primary neurons. To quantify RNA expression in the transfected cell lines, total RNA preparations from each of the constructed cells lines will be analyzed by RT-PCR (Invitrogen) for the amount of the chaperone RNA in the presence and absence of the inducer. To verify the stability of the RNA chaperone transcripts we will use northern blot methods, as well.

Quantify Aggregate Accumulation and Determine the Effect of Expressed RNAs on Cell Viability.

We anticipate that a high level of RNA chaperone expression will facilitate aggregation of amyloid proteins. Initially, Congo red, ThT, ThS and other amyloid specific dyes will be used to study the accumulation of amyloid aggregates. Immunocytochemistry methods will be used to identify specific endogenous amyloidogenic proteins, such as tau, Aβ, α-synuclein, etc, that usually participate in the formation of amyloid aggregates in neurological diseases. Cell viability of induced and non-induced cells will be assayed by lactate dehydrogenase (LDH) cytotoxicity assay (Promega).

Criteria to Progress to SBIR Phase II

In order to progress from Phase I to Phase II in this project, we will need to establish feasibility by demonstrating that the cell line containing the RNA chaperone driven by an inducible promoter is able to produce 3× the baseline amount of aggregates (and cells with pathology identified) in the brain of patients afflicted with HNDs. This will demonstrate that the synthetic RNA facilitator is both controllable and active within a mammalian cell line and therefore will justify investing the time, money and other resources to construct a transgenic mouse bearing the same construct for future testing of hits identified with this novel screening strategy. Pitfalls. Limitations and Alternative Strategies Infection with adenovirus vectors can lead to cell death if the multiplicity of infection (M.O.I) is too high for the cell type in use. This problem will be eliminated by determining the optimal M.O.I for our neuronal cultures using the β-gal containing response vector. An M.O.I that allows efficient infection of the cultures without toxicity will be selected for the subsequent experiments involving the chaperone RNAs. A known disadvantage of the tet inducible system is its sensitivity to the genome cointegration of its two vector components This results in an increase of the basal level of expression of the transgene. It has been shown previously that this problem can be overcome by delivering both vectors in two independent steps. Thus, sequential infections and selection for each of the vectors will be performed to generate the double stable, chaperone RNA inducible lines. Similarly, when constructing the mice, two transgenic mouse lines will be generated, one carrying the regulatory and the other carrying the response vector. Subsequent crossing of these two lines will generate the double, tet inducible chaperone RNA mice.

### Example 3

### Construction of a Vector for Drosophila Melanogaster

The pUC plasmid was modified to incorporate a DNA construct comprising the Drosophila heat shock promoter, the DNA for RQ 11+12, linked to a gene for white eyes in a manner known in the art. This vector is multiplied in E. coli and harvested in the manner known to individuals skillful in art.

### Example 4

### Transforming Drosophila Melanogaster Cells

The vector was injected by microinjection techniques into Drosophila melanogaster eggs. The eggs were incubated and Drosophila exhibiting white eyes are identified. These Drosophila were allowed to reproduce. Eggs from such Drosophila are divided into control and experimental groups. The experimental groups are subjected to heat shock during different stages of development. The control groups were not subjected to heat shock.

### Example 5

### Monitoring Transformed Drosophila Melanogaster for Behavior Changes

Drosophila melanogaster testing is performed after a period has passed for the non-coding RNA to cause an effect. Five days is generally sufficient. The transformed Drosophila expressing the non-coding RNA exhibit several behavior abnormalities. The Drosophila melanogaster expressing the non-coding RNA exhibit impaired learning ability to differentiate receptive female flies, reduced memory and incorrect mating sounds.

### Example 6

### Evaluation of Tissues for Agglomeration

Expression the non-coding RNA is confirmed by Northern blot in the manner known in the art. Brain tissue from Drosophila melanogaster is obtained from control flies and experimental flies. The flies expressing the non-coding RNA exhibit plaque formation in neuronal structures of the brain. Amyloid deposits are highlighted with Congo red stain revealed under photomicroscope. Other tissues are evaluated in a similar manner for agglomerations.

### Example 7

### Identification of Drug Candidates

Transformed Drosophila melanogaster are placed in two groups. One group is subjected to potential drug candidates by placing such candidate in water and food stuff. The other is a control. In the event the flies of the experimental group exhibit fewer pathologies or impairments, such group may have been subjected to a composition useful as a drug for the treatment of agglomeration disease.

### Example 8

### Construction of Trangene for Mouse

A plasmid would be modified to incorporate a DNA construct comprising a mammalian promoter, the DNA for RQ 11+12, in a manner known in the art. This vector is multiplied in E. coli and harvested in the manner known to individuals skillful in art.

### Example 9

### Transforming Mouse Cells

The vector would be injected by microinjection techniques into the pronucleus one day male mouse embryos. The eggs would be implanted into the oviduct of psuedopregnant foster female mice. The transformed mice embryos are matured into pups and born. Mice exhibiting the genotype are identified by Southern Blot analysis of the mice tissue. These mice would be allowed to reproduce.

### Example 10

### Monitoring Transformed Mouse for Behavior Change

The transformed mice would be monitored for behavior changes. The behavior changes would comprise memory loss and other mental impairment.

### Example 11

### Evaluation of Tissues for Agglomeration

Tissues from the transformed mice would be evaluated. The tissue would be examined for plaque formation in brain tissue of the type found in spongiform encephalopathies.

### Example 12

### Identification of Drug Candidate

Mice exhibiting features of agglomeration disease would be subjected to drug candidates. Compositions which altered the progression of the disease compared to control animals, would be drug candidates.

### Example 13

Q-Cartridges and Kits

A method of separating an agglomeration protein from a mixture of biological materials held in an aqueous solution potentially comprising such agglomeration protein, was performed. An immobilized phase comprising oligo dT cellulose matrix was wetted with an aqueous solution by dispersing the cellulose in the solution. The oligo dT cellulose had poly dT in a length of twenty-five nucleotides.

The aqueous solution held:

1,000× dilution of 30% Normal Sheep Brain Homogenate, 50 ul of Matrix, 10 ml reaction volume
5,000× dilution of 30% Normal Sheep Brain Homogenate, 50 ul of Matrix, 10 ml reaction volume
10,000× dilution of 30% Normal Sheep Brain Homogenate, 50 ul of Matrix, 10 ml reaction volume

Binding conditions were imposed on the wetted immobilized phase to form an binding product in the presence of the agglomeration protein. The binding conditions were physiological salt concentrations and normal pH.

The immobilized phase was separated from the aqueous solution to form a separation product which in the presence of agglomeration protein is a binding product. The binding product in the form of the normal noninfectious prion related protein (PrPc) was quantified. The amount of PrP in 1 ul 30% N. Sheep B. H. was approximately 65 ng. For the dilutions of 1,000×, 5,000× and 10,000×, the amount of PrPc was 382 ng, 70 ng, 32 ng.

The mount of PrP eluted from 50 ul Cellulose Matrix could be determined only for column processed with 1,000× dilution. This amount was approximately 15 ng. For higher dilutions, recorded data was below background value and the amount of PrP could not be determined.

Filtration Cartridges

Oligo(dT)-cellulose beads (50 mg; Sigma) and 15 pmol of gel-purified RQAAA RNA (RQ11+12 with 15 adenosines added to the 3′ end during transcription) were incubated in buffer I (10 mM Tris/HCl, pH 7.5, 500 mM NaCl and 0.5% SDS) for 30 min at 25° C. The solution was then poured into a Nanosep MF 0.2 lM filtration column (Pall). Cartridges prepared by this method could be stored for at least 4 months at 4° C. with no detectable loss in activity. After a brief spin, the column cartridge was then washed with 10 vol. of buffer 11 (10 mM Hepes, pH 8.0, 100 mM LiCl, 0.1 mM EDTA and 1 mM DTT). Samples were loaded on to cartridges, incubated for 30 min at 25° C., and washed with 1 ml of buffer II. (We routinely added 10 ml of solution to the cartridges, although the maximum volume that could be added was not determined). After washing, fractions were collected (L, loaded material; B, bound material; F, flow through) for standard Western analysis using 4-20% gradient gels (Novex). Material was removed from the column (B) by boiling in sample buffer (2 mM DTT/0.5% SDS) and collected in a volume of either 10 μl or 1 ml. The antibodies used in this study were either monoclonal antibody 3F-4, which is directed against amino acids 109-113 and is reactive to feline, hamster and human PrP (67), or monoclonal antibody 7A12, which is directed against a non-linear epitope between amino acids 90 and 145 and is reactive to mouse and human PrP (58).

Immobilized RNA binds PrPC and PrPSc

One of the limitations of current BSE diagnostics is their lack of sensitivity. Typically, 10 ng of material are required to generate a positive signal by standard Western analysis, and 10-100 pg for colorimetric assays utilizing fluorescently labelled antibodies. Assays are also limited by the amount of material that can be examined; typically less than 300 μl for microplate assay and less than 30 μl for Western analysis. Therefore, post-mortem analysis of tissue where prions are known to collect, such as brain, is required because the concentrations of PrPSc in biological fluids like blood, urine or saliva are too low to detect. However, problems related to the volume of samples could be alleviated if a PrP concentration device were developed.

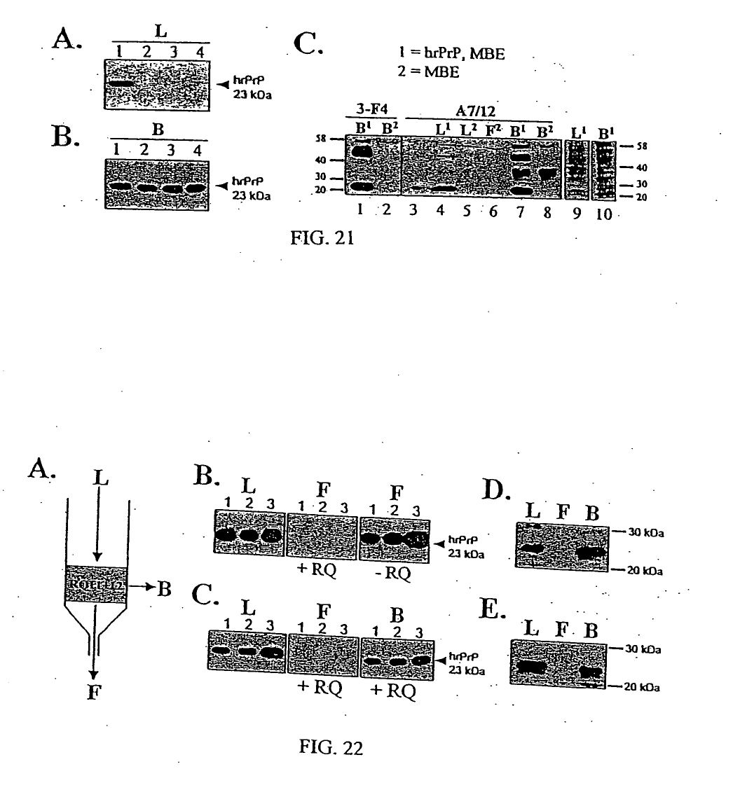

To examine if RNA could be used to bind PrP proteins from biological solutions, we developed a column cartridge containing an adsorbent impregnated with RNA. We chose to work with RQ11+12 as a representative RNA since RQ11+12 demonstrated high-affinity specific binding to hrPrP. RQ11+12 was synthesized with a 15 nt poly(A+) tail (RQAAA) and then hybridized to oligo(dT)-cellulose beads. The slurry was poured into a filter column containing a 0.2 μM membrane which was suitable for a volume of ˜1 ml of sample. Multiple loadings allowed for the examination of at least 10 ml of sample solution. Samples of the material loaded on to the column (L), retrieved from the RQAAA adsorbent (B) and the material that flowed through the column (F) were examined by Western blot analysis (FIG. 22A).

The RNA cartridges demonstrated the ability to effectively bind hrPrP from serum and urine. Solutions containing 10% bovine calf serum were spiked with increasing amounts of hrPrP and loaded on to the column to test for binding to adsorbent with or without RQAAA (FIG. 22B). Western blot analysis of the material that flowed through the column clearly indicated that RNA was necessary for the filtration effect. When the adsorbent was impregnated with RNA, there were no detectable amounts of hrPrP in the flow through (F; FIG. 22B, middle panel), indicating efficient binding to the column cartridge. However, if RQAAA was omitted from the adsorbent, virtually all of the hrPrP flowed through the column (FIG. 22B, right-hand panel). These results demonstrate that RQAAA RNA is responsible for the PrP-binding activity of the cartridge.

The cartridge was also effective for binding hrPrP from urine (FIG. 22C). Increasing amounts of hrPrP were spiked into a solution containing 10% urine and passed through a column cartridge containing the RQAAA adsorbent. A clear signal is generated from 10 μl of sample loaded on to the column (L; FIG. 22C, left-hand panel). There were no detectable amounts of hrPrP in 10 ll of flow through (F; FIG. 22C, middle panel), as apparently all of the detectable material was bound to the adsorbent (B; FIG. 22C, righthand panel).

The RNA column cartridge was also tested for binding to PrP^{Sc} derived from scrapie-infected mice. Bovine calf serum spiked with scrapie extract was passed through the column, analysed by Western blot, and PrP^{Sc} could be detected in the loaded (L) and bound (B) fractions, but not in the flow through (F; FIG. 22D). Similar data was obtained when human urine was spiked with the same scrapie extract; easily detectable signals were obtained in the loaded (L) and bound (B) fractions (FIG. 22E). In this experiment, however, a small trace signal can be observed in the flow-through fraction (F), perhaps due to a slight decrease in affinity for the PrP^{Sc} isoform. Extracts from scrapie-infected hamster gave similar results (not shown). Significantly, extensive proteinase K digestion was used in the preparation of the scrapie extract to remove the protease-sensitive PrP^{C} isoform. Thus, RNA adsorbent bound PrP^{Sc} from proteinase K-treated biological samples, warranting further investigation of the practical applications of this technology.

RNA Cartridges Increase the Limits of Detection of PrP 1000-Fold

It is possible to load 10 ml of sample on to a single PrP column and remove the material bound to the adsorbent in volumes as small as 10 μl; therefore, the RNA column should be able to serve as a PrP-concentration device. Four solutions of 10% serum were prepared with decreasing concentrations of spiked hrPrP, ranging from 1 ng/μl to 1 pg/μl. 10 μl of each solution were analysed by Western blot prior to passage through RQ11+12 adsorbent (FIG. 23A). At these concentrations, a signal could be detected only at the highest concentration, representing a total mass of 10 ng of hrPrP, which is near the detection limit expected using chemiluminescent Western blotting techniques. Amounts of 1 ng PrP or less were undetectable by Western blot. A volume that correlates to a total mass of 10 ng PrP for each of the four solutions was passed through the column. Material bound to the adsorbent was removed in a 10 μl volume and examined by Western blot (FIG. 23B). The material from all four solutions produced a signal equivalent in intensity to 10 ng of starting material. The RNA cartridge enabled the detection of hrPrP at concentrations that were previously undetectable. This approach effectively increased the level of sensitivity of Western blot analysis 1000-fold.

The ability of the RQ11+12 adsorbent to increase the sensitivity of Western blot analysis was applied to detect endogenous PrPC directly from mouse brain homogenates (FIG. 23C). Two test solutions containing mouse brain extract (MBE; 1.6 μg/μl) were prepared; a control solution spiked with 2 ng/μl hrPrP (solution 1) and a test solution containing just MBE (solution 2). Each solution (400 μl) was passed through columns containing the RQAAA adsorbent and analysed as above. A sample of solution 1 removed from the column (B1; FIG. 23C, lane 1) produced two major bands corresponding to hrPrP monomer (23 kDa; FIG. 23C, compare lanes 1 and 3) and dimer (50 kDa), which we routinely observe at higher concentrations (>20 ng} sample). A sample of solution 2 removed from the column (B2; FIG. 23C, lane 2) produced a single band of 60 kDa that we accredit to an immune-reactive, non-specific component present in MBE that weakly binds to the RNA column. This background signal is present in all samples containing MBE, irrespective of the monoclonal antibody used. The absence of signal around 35 kDa, the expected size for native, diglycosylated PrPC (68), is not surprising because monoclonal antibody 3F-4 does not bind to mouse PrPC (67).

To detect the presence of endogenous mouse PrPC (mPrP), the above solutions were also examined with the monoclonal antibody 7A12, which can bind to both hrPrP and mPrP (58). A 15 μl sample of solution 1 that was loaded on to the column (L1;

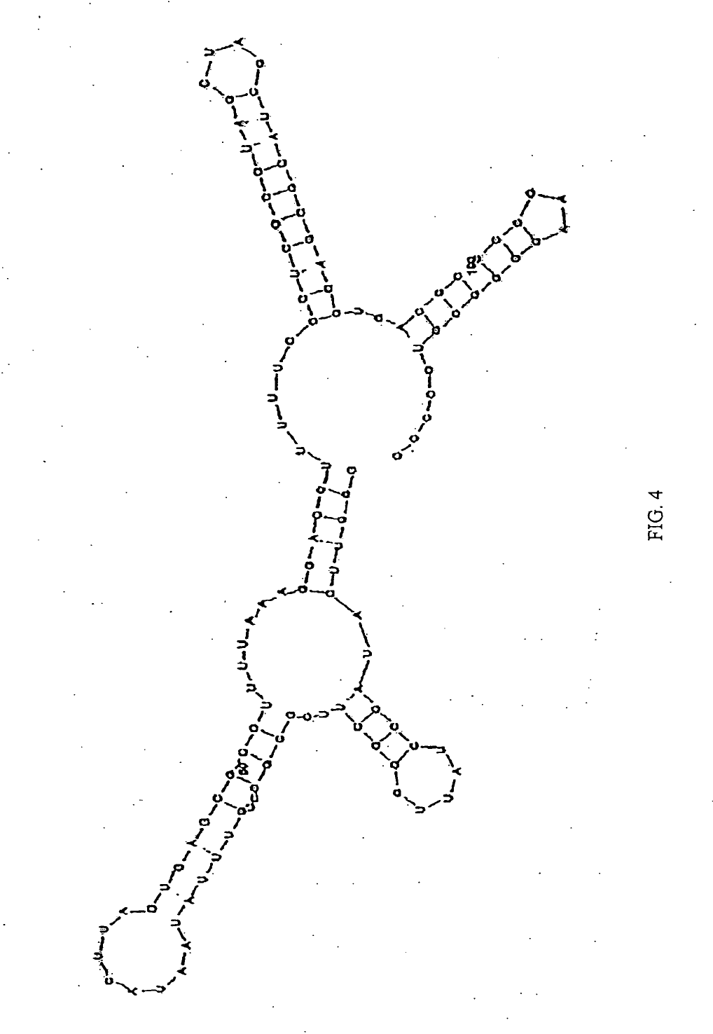

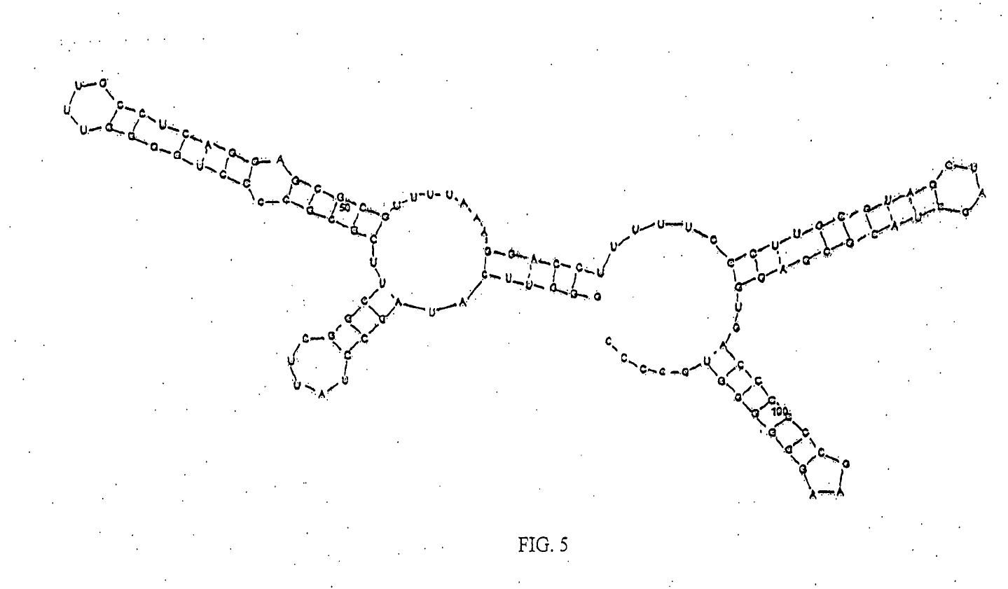

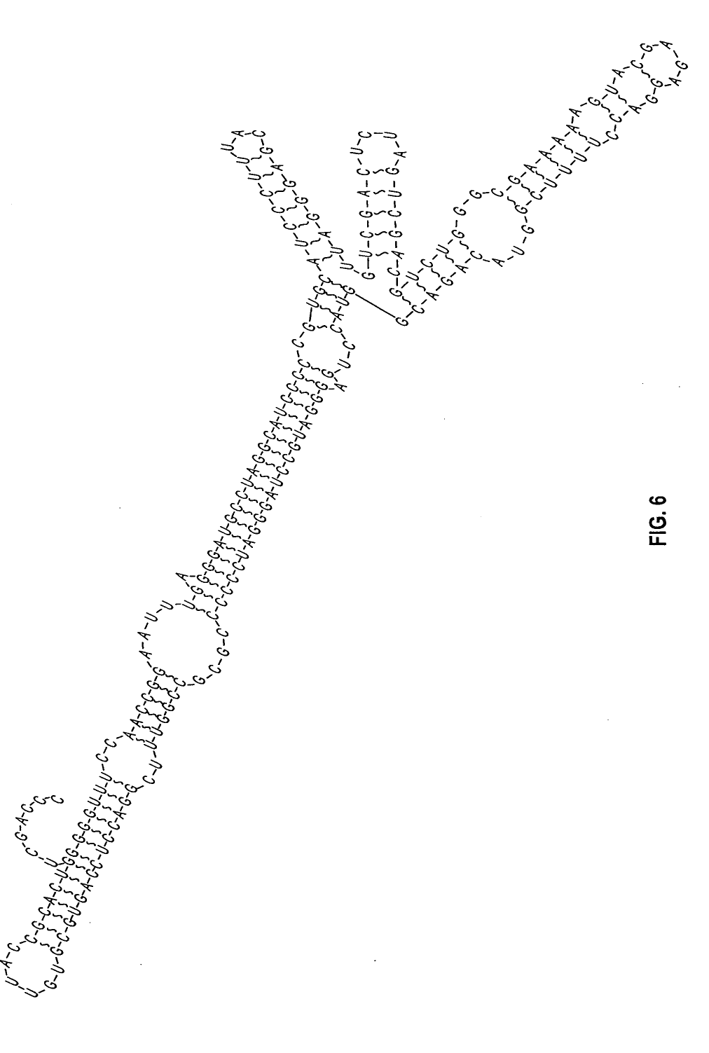

FIG. 23C, lane 4) generated the same banding pattern as observed in FIG. 23(C), lane 1, with the addition of a faint band at 35 kDa due to the presence of endogenous mPrP. This signal is greatly enhanced in the material removed from the column (B1; FIG. 23C, lane 7), demonstrating the effectiveness of the RNA column in increasing the signal from endogenous mPrP. The same treatment with solution 2 also demonstrates a tremendous increase in signal for the 35 kDa protein representing endogenous mPrP (compare L2 and B2; FIG. 23C, lanes 5 and 8). A faint doublet is also visible (B2), most likely attributable to 25 and 27 kDa forms of PrPC known to occur from partial enzymic degradation (69, 70). These truncated forms of PrPC appear to have a lower affinity for the column because their signal does not increase dramatically (compare L2 and B2; FIG. 23C, lanes 5 and 8) nor bind fully to the RNA column (compare L2 and F2; FIG. 4C, lanes 5 and 6). Coomassie Brilliant Blue staining of 10 μl samples from control solution 1 reveal that the column retains some amount of MBE bulk protein; however, it appears that enrichment is mostly for PrP proteins (FIG. 23C, lanes 9 and 10). Taken together, these data clearly demonstrate the effectiveness of the RQAAA column in increasing the sensitivity of Western detection of PrP from biological solutions.

Thus, some embodiments of the present invention pertains to amplifiable nucleic acid antibodies that have affinity for agglomeration proteins. The invention also pertains to methods of using the compositions of the present invention to interact with such proteins. Further, a kit for detecting the presence or absence of agglomeration proteins in a sample matrix is disclosed.

### Example 14

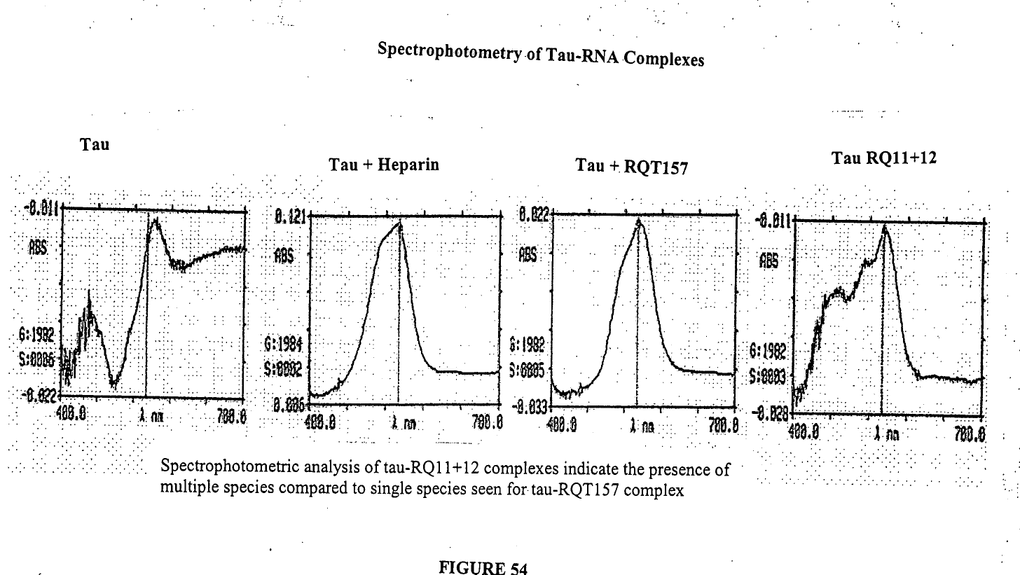

shsRNAs bind tau and conform different changes on tau. Spectrophotometry of different Tau-RNA complexes, shows that different shsRNAs bind tau and result in the formation of different species of tau-RNA complexes. See FIG. 54.

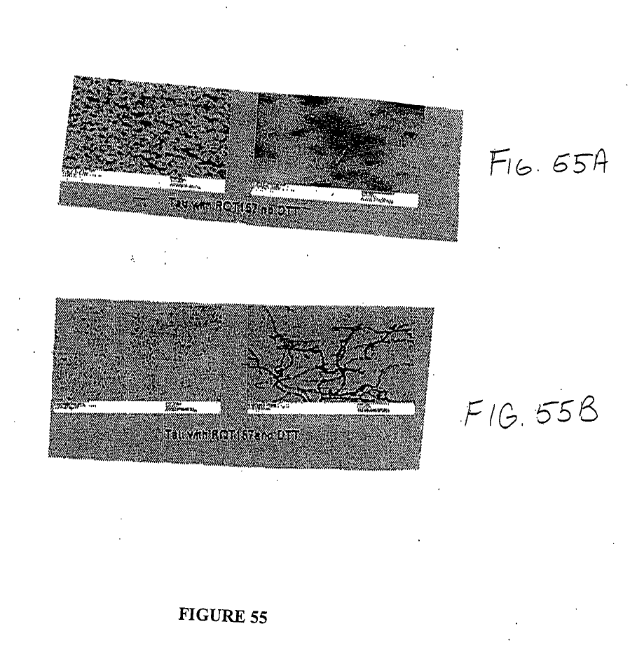

Futhermore, electron microscopy confirms this result. FIG. 55A shows formation of fibrils of tau upon addition of RQT157. Meanwhile, FIG. 55B shows the aggregation of tau upon addition of RQ11+12.

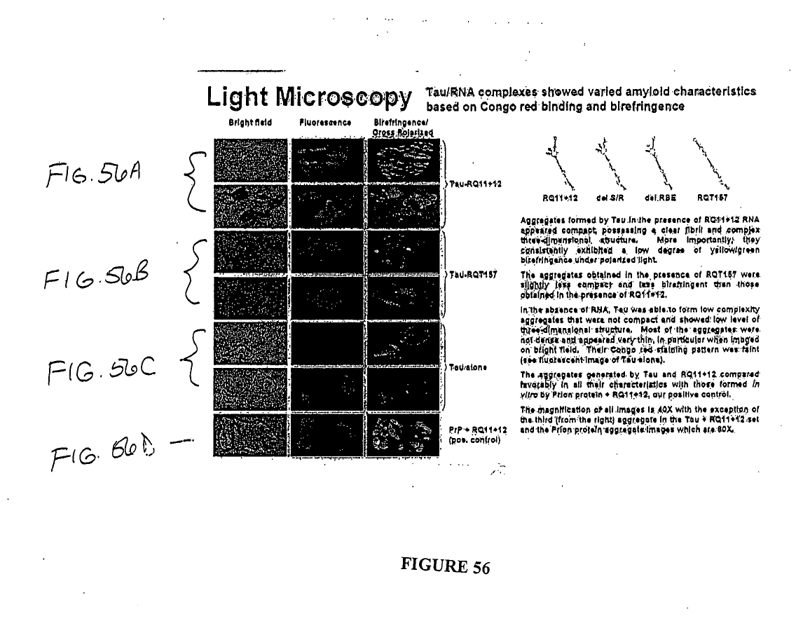

These tau-RNA complexes also showed varied amyloid characteristics based on Congo red binding and birefringence. FIG. 56A shows compact aggregates, formed by tau in the presence of RQ11+12 RNA, possessing a clear fibril and complex three-dimensional structure. They consistently exhibited a low degree of yellow/green birefringence under polarized light.

The aggregates obtained in the presence of RQT157 were slightly less compact and less birefringent than those obtained in the presence of RQ11+12. See FIG. 56B.

In the absence of RNA, tau was able to form low complexity aggregates that were not compact and showed low level of three-dimensional structure. Most of the aggregates were not dense and appeared very thin, in particular when imaged on bright field. Their Congo red staining patter was faint (see FIG. 56C showing fluorescent image of tau alone).

The aggregates generated by tau and RQ11+12 compared favorably in all their characteristics with those formed in vitro by prion protein+RQ11+12, which was used as the positive control. (See FIG. 56D).

### Example 15

The following experiments were performed to determine the necessary components of standard protection assay buffer are needed for RNA-Tau aggregation:

Preparation of 1× Protection Assay Buffer:

Combine the following:

20 mM HEPES pH 7.4

1 mM EGTA

1 mM DTT

0.5% NP-40

2.5% Glycerol

50 mM NaCl

Materials:

RQ11+12˜1 ug/ul, purified on 491 Prep Cell BIO-RAD

Tau 1 ug/ul purified on 491 Prep Cell BIO-RAD

1×TBE running buffer

6× Mass Ruler Loading Dye

Dilutions:

Use RQ, Tris and NaCl dilutions

Procedure:

Prepare using standard and modified 10× Protection Assay Buffers. Incubate for 30 minutes. Run 18 well, 12.5% Tris-HCl BIO-RAD precast gel.

TABLE 8
10X Modified Protection Assay Buffers, 1 ml final volume:

| | | 10X | | 10X | | 10X | |
|---|---|---|---|---|---|---|---|
| | | PAB | 10X | PAB | 10X | PAB | |
| | | No | PAB | No NP- | PAB | No | 10X PAB |
| Stocks | 10X PAB | NaCl | No Glyc. | 40 | No DTT | EGTA | No HEPES |
| 1M HEPES | 200 ul | 200 ul | 200 ul | 200 ul | 200 ul | 200 ul | 200 ul Tris |
| | | | | | | | 1M pH 7.4 |
| 0.5M EGTA | 20 ul | 20 ul | 20 ul | 20 ul | 20 ul | — | 20 ul |
| 1M DTT | 5 ul | 5 ul | 5 ul | 5 ul | — | 5 ul | 5 ul |
| 20% NP-40 | 250 ul | 250 ul | 250 ul | — | 250 ul | 250 ul | 250 ul |
| 100% | 250 ul | 250 ul | — | 250 ul | 250 ul | 250 ul | 250 ul |
| Glycerol | | | | | | | |
| 5M NaCl | 100 ul | — | 100 ul | 100 ul | 100 ul | 100 ul | 100 ul |
| dH₂O | 175 ul | 275 ul | 425 ul | 425 ul | 180 ul | 195 ul | 175 ul |

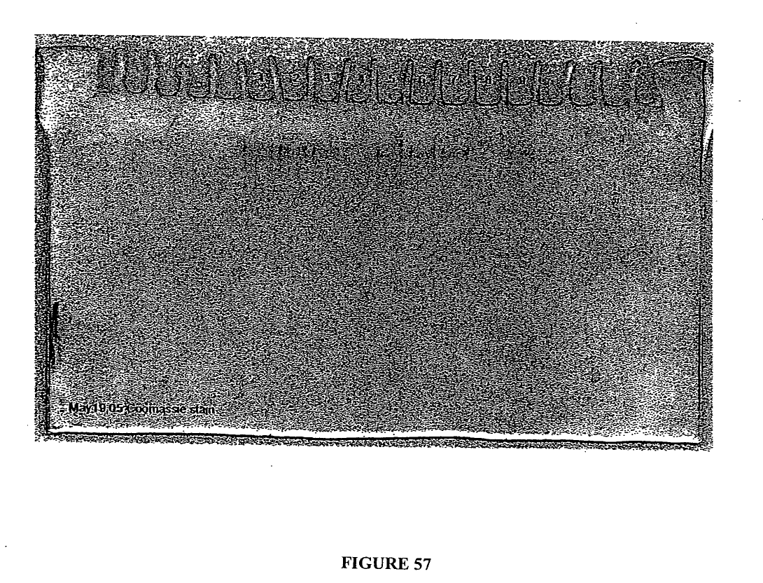

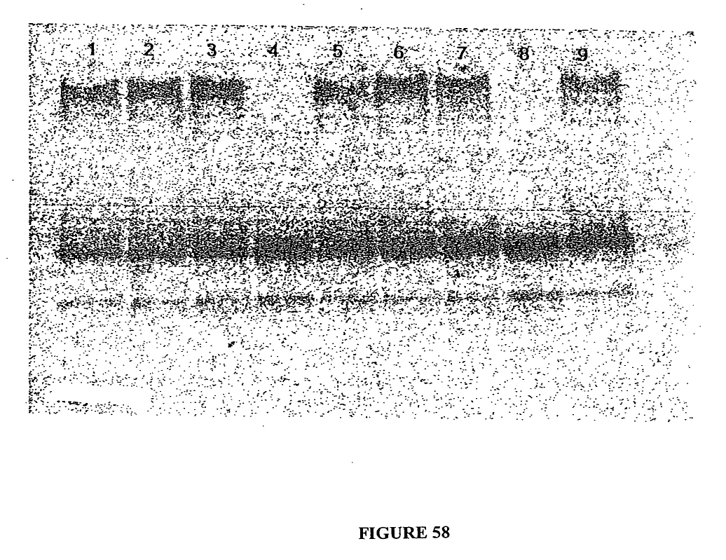

Protocol:
For each of the Buffers listed in Table 8, set up 1, 10 ul reaction containing: 1 ug Tau, 125 ng RQ11+12, Final concentration of Buffer l×.
Use 1 ul Tau (1 ug/ul), 1 ul RQ11+12 (125 ng/ul), 1 ul 10×PAB or its modification, 7 ul dH₂O.
Include positive control for RQ alone and standard 1×PAB.
See FIGS. 57 and 58 for Gel Assay results, wherein:
Lane 1=1× Protection Assay Buffer pH 7.4
Lane 2=1× Protection Assay Buffer pH 7.4 No NaCl
Lane 3=1× Protection Assay Buffer pH 7.4 No Glycerol
Lane 4=1× Protection Assay Buffer pH 7.4 No NP-40
Lane 5=1× Protection Assay Buffer pH 7.4 No DTT
Lane 6=1× Protection Assay Buffer pH 7.4 No EGTA
Lane 7=1× Protection Assay Buffer pH 7.4 No HEPES (Use 20 mM Tris-HCl pH 7.4 instead)
Lane 8=RQ11+12
Lane 9=1× Protection Assay Buffer pH 7.4
Results:
Non-ionic detergent NP-40 is necessary in protection assay buffer for RQ-Tau aggregation (see lanes 4 of FIGS. 57 and 58).
Removal of other components: NaCl, Glycerol, DTT, EGTA, HEPES did not produce similar results

### Example 16

The following experiment was performed to determine RNA-Tau aggregation under conditions of decreasing amount of NP-40 in Protection Assay Buffer:

Preparation of 1× Protection Assay Buffer:

Combine the following:

20 mM HEPES pH 7.4

1 mM EGTA

1 mM DTT

0.5% NP-40

2.5% Glycerol

50 mM NaCl

Materials:

RQ11+12˜1 ug/ul purified on 491 Prep Cell BIO-RAD

Tau 1 ug/ul purified on 491 Prep Cell BIO-RAD

1×TBE running buffer

6× Mass Ruler Loading Dye

TABLE 9
Prepare 10X Modified Protection Assay Buffers in 1 ml final volume
containing different concentration of NP-40:

| | Nr | | | |
|---|---|---|---|---|
| | 3 | 4 | 5 | 6 |
| Stocks | 10X PAB | 10X PAB | 10X PAB | 10X PAB |
| | No NP-40 | 1% NP-40 | 2% NP-40 | 5% NP-40 |
| 1M HEPES | 200 ul | 200 ul | 200 ul | 200 ul |
| 0.5 M EGTA | 20 ul | 20 ul | 20 ul | 20 ul |
| 1M DTT | 5 ul | 5 ul | 5 ul | 5 ul |
| 20% NP-40 | — | 50 ul | 100 uol | 250 ul |
| 100% Glycerol | 250 ul | 250 ul | 250 ul | 250 ul |
| 5M NaCl | 100 ul | 100 ul | 100 ul | 100 ul |
| dH₂O | 425 ul | 375 ul | 325 ul | 175 ul |

Gel Set Up:

| Nr | Sample |
|---|---|
| 1 | Tau, RQ, 1x Protection Assay Buffer pH 7.4 |
| 2 | Tau, RQ, 1x Protection Assay Buffer pH 7.4 No NP-40 |
| 3 | Tau, RQ, 1x Protection Assay Buffer pH 7.4 No NP-40 |
| 4 | Tau, RQ, 1x Protection Assay Buffer pH 7.4 0.1% NP-40 |
| 5 | Tau, RQ, 1x Protection Assay Buffer pH 7.4 0.2% NP-40 |
| 6 | Tau, RQ, 1x Protection Assay Buffer pH 7.4 0.5% NP-40 |
| 7 | Tau, 1x Protection Assay Buffer pH 7.4 |
| 8 | Tau, 1x Protection Assay Buffer pH 7.4 No NP-40 |
| 9 | Tau, 1x Protection Assay Buffer pH 7.4 No NP-40 |
| 10 | Tau, 1x Protection Assay Buffer pH 7.4 0.1% NP-40 |
| 11 | Tau, 1x Protection Assay Buffer pH 7.4 0.2% NP-40 |
| 12 | Tau, 1x Protection Assay Buffer pH 7.4 0.5% NP-40 |
| 13 | RQ, 1x Protection Assay Buffer pH 7.4 |
| 14 | RQ, 1x Protection Assay Buffer pH 7.4 No NP-40 |
| 15 | RQ, 1x Protection Assay Buffer pH 7.4 No NP-40 |
| 16 | RQ, 1x Protection Assay Buffer pH 7.4 .01% NP-40 |
| 17 | RQ, 1x Protection Assay Buffer pH 7.4 0.2% NP-40 |
| 18 | RQ, 1x Protection Assay Buffer pH 7.4 0.5% NP-40 |

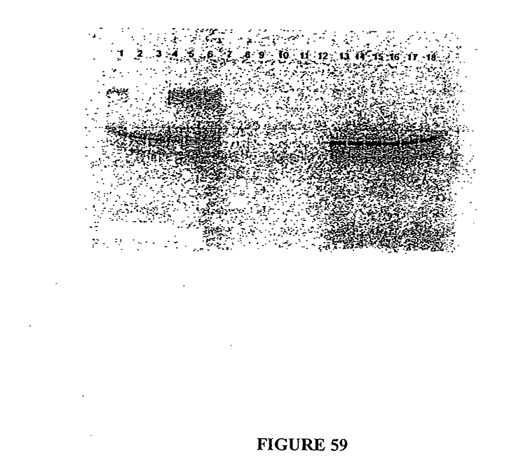

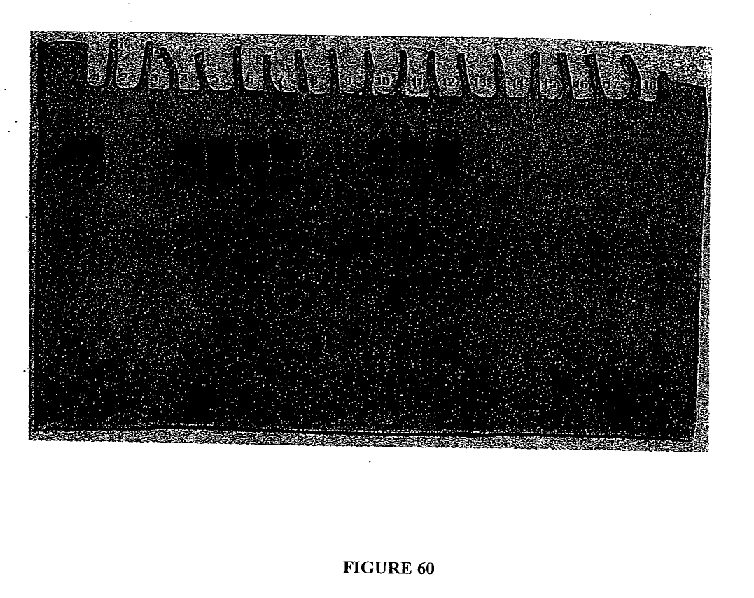

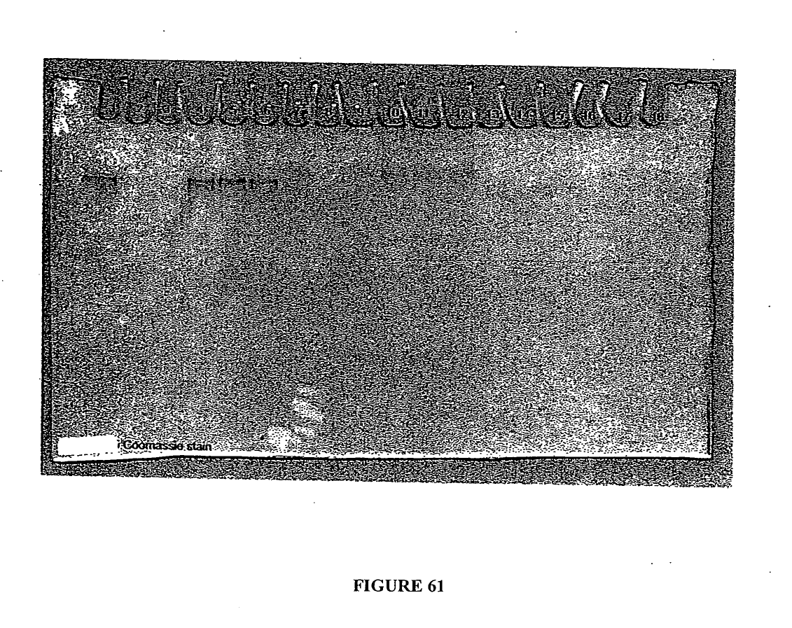

Procedure:
Set up three sets of reactions containing:
SET 1: 1 ug Tau and 125 ng RQ in 1× Protection Assay Buffer (1, 2, 3, 4, 5 or 6)
Mix reagents in given order: 7 ul dH₂O, 1 ul 10× Protection Assay Buffer (1, 2, 3, 4, 5 or ul Tau, 1 ul RQ
SET 2: 1 ug Tau in 1× Protection Assay Buffer (1, 2, 3, 4, 5 or 6)
Mix reagents in given order: 8 ul dH₂O, 1 ul 10× Protection Assay Buffer (1, 2, 3, 4, 5 or ul Tau
SET 3: 125 ng RQ in 1× Protection Assay Buffer (1, 2, 3, 4, 5 or 6)
Mix reagents in given order: 8 ul dH₂O, 1 ul 10× Protection Assay Buffer (1, 2, 3, 4, 5 or ul RQ
Incubate reactions from all sets for 30 minutes at room temperature.
Add 4 ul 6× Mass Ruler Loading Dye. Do not boil.
Load on 12.5% Tris-HCl precast gel BIO-RAD Critarion.
Run gel at 80V for 30 minutes and 230V for 60 minutes.
Keep gel box on ice.
Stain gel with SybrGold for 15 minutes. Visualize bands on transilluminator. Take a picture.
Stain gel with Gel Code Pierce Coomassie dye for 1 h, distain with water over the weekend. Scan gel on the scanner. Rinse with water.
Stain with Silver Stain following Pierce protocol. Scan gel on the scanner.
Results:
See FIGS. 59, 60 and 61 for gel assay results.
Reduction of the amount of NP-40 from concentration 0.5% to 0.2% and 0.1% did not have significant effect on RQ-Tau aggregation. (See lanes 6, 4, 5, respectively.)

### Example 17

The following experiments were performed to determine whether aggregation takes place in the presence of 20 mM Tris-HCl pH 7.4:

The experiment was prepared in two sets:

(1) Using 20 mM Tris-HCl pH 7.4 as a “base” buffer for set 2.
(2) Using 1× Protection Assay Buffer pH 7.4 without LiCL as a “base” buffer for set 1.
   Run both sets on 18 well, 12.5% Tris-HCl BIO-RAD precast gel.
   Materials:
   RQ11+12˜1 ug/ul purified on 491 Prep Cell BIO-RAD
   Tau 1 ug/ul purified on 491 Prep Cell BIO-RAD
   Tris-HCl 1M Sigma pH 7.4
   NaCl 5M Sigma
   10× Protection Assay Buffer pH 7.4 (No LiCl)
   1×TBE running buffer
   6× Mass Ruler Loading Dye
   Dilutions:
   Dilute: RQ stock 8× in dH₂O to final concentration 0.125 ug/ul in 48 ul final volume
   Mix: 6 ul RQ and 42 ul dH₂O
   Dilute: Tris-HCl pH 7.4 stock 5× in dH₂O to final concentration 200 mM in 500 ul final volume
   Mix: 100 ul Tris-HCL 1M and 400 ul dH₂O
   Dilute: NaCl stock 5× in dH₂O to final concentration 1 M in 500 ul final volume
   Mix: 100 ul 5M NaCl and 400 ul dH₂O
   Dilute: NaCl stock 50× in dH₂O to final concentration 100 mM final volume
   Mix: 20 ul 5M NaCl and 980 ul dH₂O
   Protocol for Set 1:

Mix reagents in given order: water, NaCl, Buffer, Tau, RQ

| | | | | 10x | | |
|---|---|---|---|---|---|---|
| | | | NaCL | PAB | | |
| Sample | | | 100 mM | No | Tau | RQ |
| Nr. | Sample description | H₂O | or 1M | LiCl | 1 ug/ul | 0.125 ug/ul |
| 1 | 0.125 ug RQ Positive Control | 9 ul | — | — | — | 1 ul |
| 2 | 1 ug Tau, 0.125 ug RQ, 1xPAB LiCl—, 10 mM | 6 ul | 1 ul | 1 ul | 1 ul | 1 ul |
| | NaCl | | | | | |
| 3 | 1 ug Tau, 0.125 ug RQ, 1xPAB LiCl—, 25 mM | 4.5 ul | 2.5 ul | 1 ul | 1 ul | 1 ul |
| | NaCl | | | | | |
| 4 | 1 ug Tau, 0.125 ug RQ, 1xPAB LiCl—, 50 mM | 2 ul | 5 ul | 1 ul | 1 ul | 1 ul |
| | NaCl | | | | | |
| 5 | 1 ug Tau, 0.125 ug RQ, 1xPAB LiCl—, 75 mM | 6.25 ul | 0.75 ul | 1 ul | 1 ul | 1 ul |
| | NaCl | | | | | |
| 6 | 1 ug Tau, 0.125 ug RQ, 1xPAB LiCl—, 100 mM | 6 ul | 1 ul | 1 ul | 1 ul | 1 ul |
| | NaCl | | | | | |
| 7 | 1 ug Tau, 0.125 ug RQ, 1xPAB LiCl—, 150 mM | 5.5 ul | 1.5 ul | 1 ul | 1 ul | 1 ul |
| | NaCl | | | | | |
| 8 | 1 ug Tau, 0.125 ug RQ, 1xPAB LiCl—, 200 mM | 5 ul | 5 ul | 1 ul | 1 ul | 1 ul |
| | NaCl | | | | | |
| 9 | 1 ug Tau, 0.125 ug RQ, 1xPAB LiCl—, 10 mM | 4 ul | 3 ul | 1 ul | 1 ul | 1 ul |
| | NaCl | | | | | |

Protocol for Set 2:

Mix reagents in given order: water, NaCl, Buffer, Tau, RQ

| | | | | 10x | | |
|---|---|---|---|---|---|---|
| | | | NaCL | PAB | | |
| Sample | | | 100 mM | No | Tau | RQ |
| Nr. | Sample description | H₂O | or 1M | LiCl | 1 ug/ul | 0.125 ug/ul |
| 10 | 1 ug Tau, 0.125 ug RQ, 20 mM Tris-HCl, 10 mM | 6 ul | 1 ul | 1 ul | 1 ul | 1 ul |
| | NaCl | | | | | |
| 11 | 1 ug Tau, 0.125 ug RQ, 20 mM Tris-HCL, 25 mM | 4.5 ul | 2.5 ul | 1 ul | 1 ul | 1 ul |
| | NaCL | | | | | |
| 12 | 1 ug Tau, 0.125 ug RQ, 20 mM Tris-HCL, 50 mM | 2 ul | 5 ul | 1 ul | 1 ul | 1 ul |
| | NaCL | | | | | |
| 13 | 1 ug Tau, 0.125 ug RQ, 20 mM Tris-HCL, 75 mM | 6.25 ul | 0.75 ul | 1 ul | 1 ul | 1 ul |
| | NaCL | | | | | |
| 14 | 1 ug Tau, 0.125 ug RQ, 20 mM Tris-HCL, 100 mM | 6 ul | 1 ul | 1 ul | 1 ul | 1 ul |
| | NaCL | | | | | |
| 15 | 1 ug Tau, 0.125 ug RQ, 20 mM Tris-HCL, 150 mM | 5.5 ul | 1.5 ul | 1 ul | 1 ul | 1 ul |
| | NaCL | | | | | |
| 16 | 1 ug Tau, 0.125 ug RQ, 20 mM Tris-HCL, 200 mM | 5 ul | 2 ul | 1 ul | 1 ul | 1 ul |
| | NaCL | | | | | |
| 17 | 1 ug Tau, 0.125 ug RQ, 20 mM Tris-HCL, 300 mM | 4 ul | 3 ul | 1 ul | 1 ul | 1 ul |
| | NaCL | | | | | |
| 18 | 1 ug Tau, 0.125 ug RQ, 1xPAB with LiCl, No NaCl | 7 ul | — | 1 ul | 1 ul | 1 ul |
| | Positive Control | | | 10X | | |
| | | | | PAB | | |

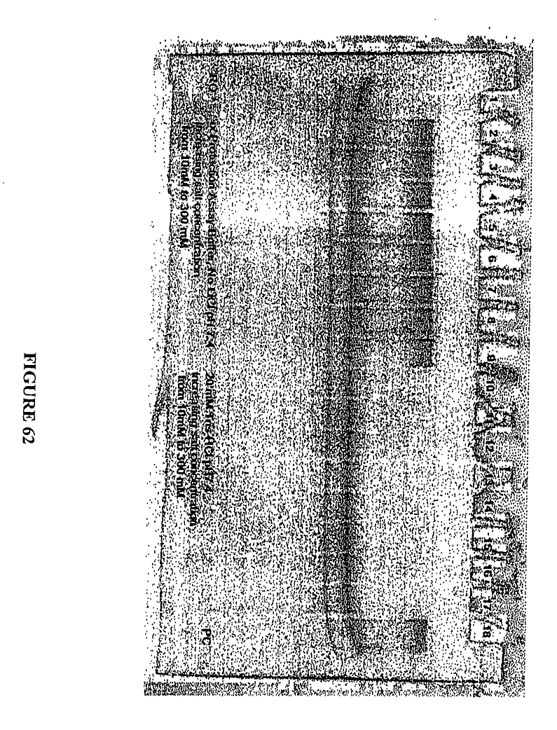

Incubate the reaction from both sets for 30 minutes at room temperature.
Add 4 ul 6× Mass Ruler Loading Dye. Do not boil.
Load on 12.5% Tris-HCl precast gel BIO-RAD Critarion.
Run gel at 80V for 20 minutes and 230V for 40 minutes.
Keep gel box on ice.
Stain gel with SybrGold for 15 minutes. Visualize bands on transilluminator. Take a picture.
Scan gel on Fluoroimager at F570 FD30.
Results:
See gel assay in FIG. 62.
Tau-RQ aggregation is observed in all samples incubated with Protection Assay Buffer. Variation in salt concentration has little effect on aggregation. Only at the highest salt concentration 200-300 mM NaCl slight decrease in amount of shifted RQ is observed. Tau-RQ aggregation is significantly inhibited when Protection Assay Buffer is replaced with 20 mM Tris-HCl pH 7.4. Inhibition of aggregation is visible even at the lowest salt concentration 10 mM NaCl. This stresses importance of Protection Assay Buffer composition for Tau-RQ aggregation.

### Example 18

Amounts of tau protein were varied vis-á-vis RQ11+12 to establish stochiometry of the tau-RQ11+12 interaction. Gel shifts of RQ11+12 with Tau23 (or “Q-RNA Tau with His tag) and Tau411 (without a Histidine tag) were compared:

| | Tau | RQ11+12 | 10x PAB | |
|---|---|---|---|---|
| No. | (1 ug/ul) | (125 ng/ul) | pH 7.4 | H₂O |
| 1 | 0 ul | 1 ul | 1 ul | 8 ul |
| 2 | 1 ul | 1 ul | 1 ul | 7 ul |
| 3 | 2 ul | 1 ul | 1 ul | 6 ul |
| 4 | 3 ul | 1 ul | 1 ul | 5 ul |
| 5 | 4 ul | 1 ul | 1 ul | 4 ul |
| 6 | 6 ul | 1 ul | 1 ul | 2 ul |
| 7 | 8 ul | 1 ul | 1 ul | 0 ul |
| 1* | 0 ul | 1 ul | 1 ul | 8 ul |
| 2* | 1 ul | 1 ul | 1 ul | 7 ul |
| 3* | 2 ul | 1 ul | 1 ul | 6 ul |
| 4* | 3 ul | 1 ul | 1 ul | 5 ul |

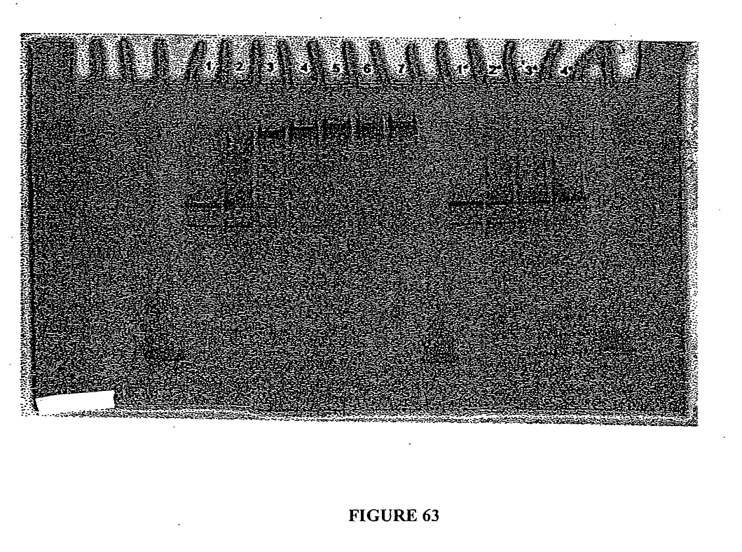

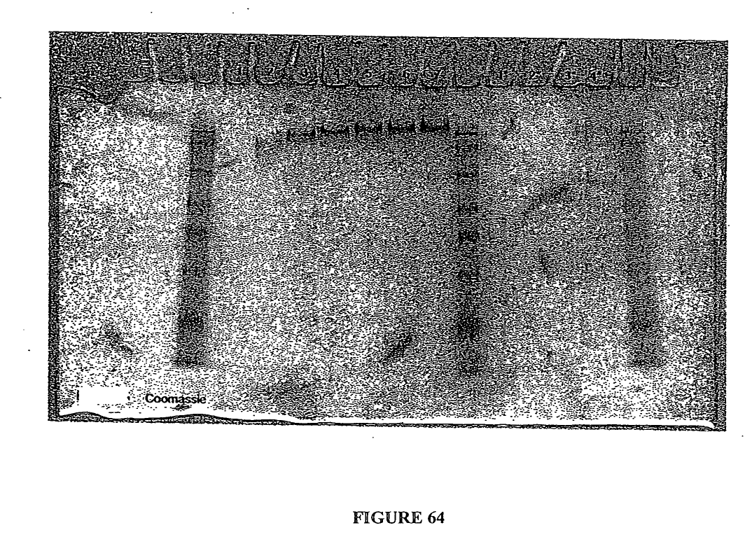

Columns 1-7 used Q-RNA's Tau and columns 1*-4*used Tau 441. Reactions were incubated for 30 minutes at room temperature, then 4 ul of 6× Mass Ruler Loading Dye was added. The samples were loaded on to a 12.5% Tris-HCl precast gel BIO-RAD Critarion. The gels were run at 80V for 20 minutes and then 230V for 40 minutes on ice. They were stained with SybrGold for 15 minutes and the bands visualized on a transilluminator. A picture was then taken. (See FIG. 63). The gels were then scanned on FluorImager 595 with filter F570FD30. The gels were then stained with Gel Code Pierce Coomassie dye for one hour, istain with water overnight and then scanned. See FIG. 64.

### Example 19

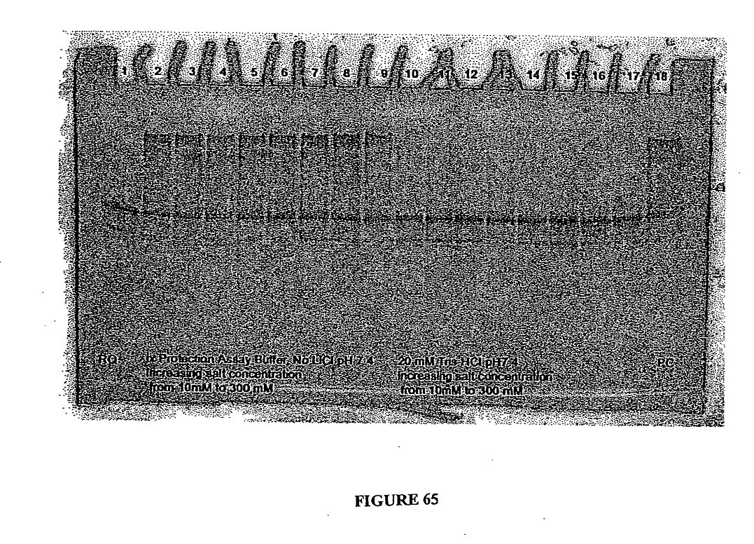

The goal of this example was to optimize the salt concentration of the Tripartite system and to determine whether aggregation occurs in the presence of 20 mM Tris-HCl pH 7.4. The samples loaded onto the gel show in FIG. 65, were prepared as follows:

| | | | NaCl | | | |
|---|---|---|---|---|---|---|
| | | | 100 mM | | | |
| Sample | | | or | | Tau | RQ |
| Nr | Sample description | H₂O | 1M | | 1 ug/ul | 0.125 ug/ul |
| | | | | 10x PAB | | |
| | | | | No LiCl | | |
| 1 | 0.125 ug RQ | 9 ul | — | — | — | 1 ul |
| | Postive Control | | | | | |
| 2 | 1 ug Tau, 0.125 ug RQ, 1xPAB LiCl—, 10 mM NaCl | 6 ul | 1 ul | 1 ul | 1 ul | 1 ul |
| 3 | 1 ug Tau, 0.125 ug RQ, 1xPAB LiCl—, 25 mM NaCl | 4.5 ul | 2.5 ul | 1 ul | 1 ul | 1 ul |
| 4 | 1 ug Tau, 0.125 ug RQ, 1xPAB LiCl—, 50 mM NaCl | 2 ul | 5 ul | 1 ul | 1 ul | 1 ul |
| 5 | 1 ug Tau, 0.125 ug RQ, 1xPAB LiCl—, 75 mM NaCl | 6.25 ul | [symbol] | 1 ul | 1 ul | 1 ul |
| 6 | 1 ug Tau, 0.125 ug RQ, 1xPAB LiCl—, 100 mM NaCl | 6 ul | [symbol] | 1 ul | 1 ul | 1 ul |
| 7 | 1 ug Tau, 0.125 ug RQ, 1xPAB LiCl—, 150 mM NaCl | 5.5 ul | [symbol] | 1 ul | 1 ul | 1 ul |
| 8 | 1 ug Tau, 0.125 ug RQ, 1xPAB LiCl—, 200 mM NaCl | 5 ul | [symbol] | 1 ul | 1 ul | 1 ul |
| 9 | 1 ug Tau, 0.125 ug RQ, 1xPAB LiCl—, 300 mM NaCl | 4 ul | [symbol] | 1 ul | 1 ul | 1 ul |
| | | | | Tris-HCl | | |
| | | | | 200 mM | | |
| 10 | 1 ug Tau, 0.125 ug RQ, 20 mM Tris-HCl, 10 mM NaCl | 6 ul | 1 ul | 1 ul | 1 ul | 1 ul |
| 11 | 1 ug Tau, 0.125 ug RQ, 20 mM Tris-HCl, 25 mM NaCl | 4.5 ul | 2.5 ul | 1 ul | 1 ul | 1 ul |
| 12 | 1 ug Tau, 0.125 ug RQ, 20 mM Tris-HCl, 50 mM NaCl | 2 ul | 5 ul | 1 ul | 1 ul | 1 ul |
| 13 | 1 ug Tau, 0.125 ug RQ, 20 mM Tris-HCl, 75 mM NaCl | 6.25 ul | [symbol] | 1 ul | 1 ul | 1 ul |
| 14 | 1 ug Tau, 0.125 ug RQ, 20 mM Tris-HCl, 100 mM NaCl | 6 ul | [symbol] | 1 ul | 1 ul | 1 ul |
| 15 | 1 ug Tau, 0.125 ug RQ, 20 mM Tris-HCl, 150 mM NaCl | 5.5 ul | [symbol] | 1 ul | 1 ul | 1 ul |
| 16 | 1 ug Tau, 0.125 ug RQ, 20 mM Tris-HCl, 200 mM NaCl | 5 ul | [symbol] | 1 ul | 1 ul | 1 ul |
| 17 | 1 ug Tau, 0.125 ug RQ, 20 mM Tris-HCl, 300 mM NaCl | 4 ul | [symbol] | 1 ul | 1 ul | 1 ul |
| 18 | 1 ug Tau, 0.125 ug RQ, 1xPAB with LiCl, No NaCl | 7 ul | [symbol] | 1 ul 10x | 1 ul | 1 ul |
| | Positive Colntrol | | | PAB | | |

All of the reactions were incubated for 30 minutes at room temperature, after which 4 ul of 6× Mass Ruler Loading Due was added. The samples were loaded onto a 12.5% Tris-HCl precast gel BIO-RAD Critarion. Gels were run at 80V for 20 minutes, followed by 230V. for 40 minutes, on ice. The gels were then stained with SybrGold for 15 minutes, and their bands visualized on a transilluminator. Pictures were taken and then the gels scanned on Fluoroimager at F570FD30. (See FIG. 65).

Tau-RQ aggregation was observed in all the samples incubated with PAB. Variation in salt concentration had little effect on aggregation. Only at the highest salt concentration of 200-300 mM NaCl was a slight decrease in the amount of shifted RQ observed.

Tau-RQ aggregation is inhibited when PAB is replaced with 20 mM Tris-HCl, pH 7.4. Inhibition of aggregation is visible even at the lowest salt concentration of 10 mM NaCl.

### Example 20

This example examines the effect of increasing salt stringency on the stochiometry of the tau-RNA complex.

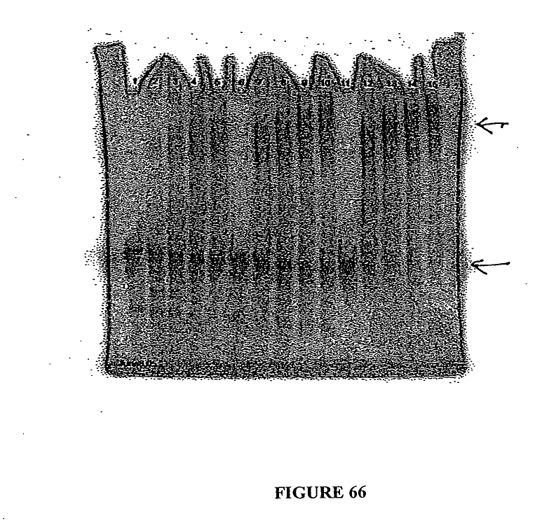

The results are shown in the gel of FIG. 66. The gels were run as follows:

| | Tau | | | | | | | |
|---|---|---|---|---|---|---|---|---|
| | (ug)/NaCl | RQ11+12 | Tau | Tau | 10x PAB | NaCl | NaCl | |
| No. | conc. | (125 ng/ul) | (1 ug/ul) | (5 ug/ul) | pH 7.4 | 5M | 1M | H₂O |
| 1 | 0/1000 | 1 ul | | | 1 ul | 2 ul | | 6 ul |
| 2 | 1/1000 | 1 ul | 1 ul | | 1 ul | 2 ul | | 5 ul |
| 3 | 2/1000 | 1 ul | 2 ul | | 1 ul | 2 ul | | 4 ul |
| 4 | 5/1000 | 1 ul | | 1 ul | 1 ul | 2 ul | | 5 ul |
| 5 | 10/1000 | 1 ul | | 2 ul | 1 ul | 2 ul | | 4 ul |
| 6 | 0/500 | 1 ul | | | 1 ul | 1 ul | | 7 ul |
| 7 | 1/500 | 1 ul | 1 ul | | 1 ul | 1 ul | | 6 ul |
| 8 | 2/500 | 1 ul | 2 ul | | 1 ul | 1 ul | | 5 ul |
| 9 | 5/500 | 1 ul | | 1 ul | 1 ul | 1 ul | | 6 ul |
| 10 | 10500 | 1 ul | | 2 ul | 1 ul | 1 ul | | 5 ul |
| 11 | 0/150 | 1 ul | | | 1 ul | | 1.5 ul | 6.5 ul |
| 12 | 1/150 | 1 ul | 1 ul | | 1 ul | | 1.5 ul | 5.5 ul |
| 13 | 2/150 | 1 ul | 2 ul | | 1 ul | | 1.5 ul | 4.5 ul |
| 14 | 5/150 | 1 ul | | 1 ul | 1 ul | | 1.5 ul | 5.5 ul |
| 15 | 10/150 | 1 ul | | 2 ul | 1 ul | | 1.5 ul | 4.5 ul |

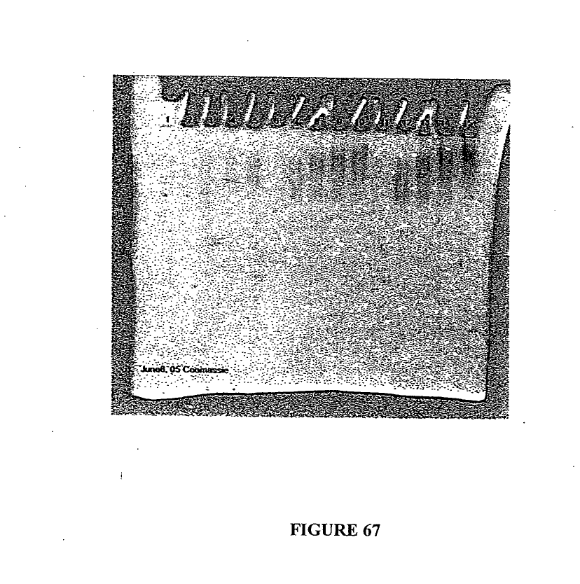

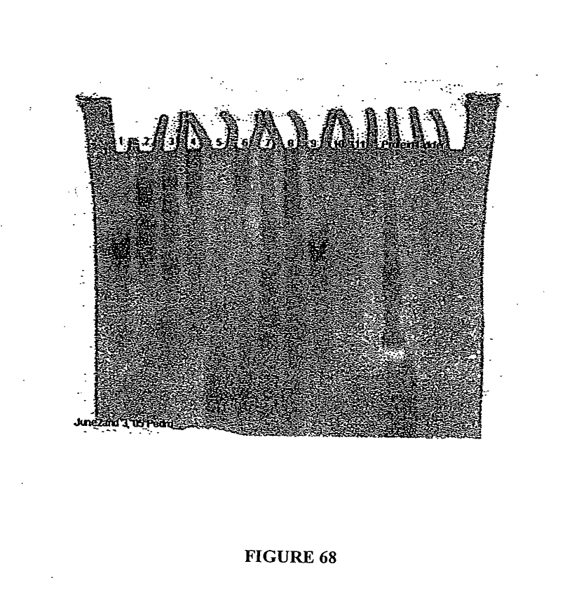

The reactions were incubated for 30 minutes at room temperature followed by the addition of 4 ul of 6× Loading Buffer Fermentas. Samples were loaded onto a 6% PA/TBE/7M urea gel Novex from Invitrogen. The gels were run on ice at 80V for 20 minutes, 150V for 10 minutes and then 2.5 hrs. at 180V, using 1×TBE as the running buffer. The gel was then stained with SYBR gold for 15 minutes, photographed and scanned on a FluorImager595 with filter F570FD30. The gels were then scanned with GelCode Coomassie stain for 1 hour, distain for 2 days with water, scanned and photographed. (See FIGS. 67 and 68)

RQ11+12/Tau interaction is maximum at physiological NaCl (150 mM) and saturating amount of protein (10 ug). Followed by a linear dissociation of RQ11+12 molecules from tau protein as NaCl molarity is increased from 150 mM to 1 M; indicating that under physiological conditions 132.5 molecules of tau protein interact with 1 molecule of RNA.

Denaturant resistant (7M urea) interaction between Tau (Q-RNA tau) and RQ11+12 exists, i.e., RQ11+12/tau complexes are stable when run in a 7M urea gel. Lanes 2-5 contain RQ11+12/Tau interactions at 1M NaCl, Lanes 7-10 contain RQ11+12/Tau interactions at 0.15M NaCl. The affinity between RQ11+12 and tau at a particular site is decreased with increasing salt, where equilibrium of binding is shifted.

### Example 21

The binding of different RNAs (RQ11+12, MNV AP1, and tRNA) to tau was compared. The binding of two different tau proteins, “tau23” and “tau441”, to these RNAs were also compared.

Tau441 (also referred to as “rPeptide Tau”) and Tau23 have certain differences:

| | | | | | Calculated |
|---|---|---|---|---|---|
| | | Purification | | | Concen- |
| Protein | His-tag | Method | Isoform | MW (kDa) | tration |
| Tau23 | 6-His | 491 Prep | Fetal/Abult | 37.7 | 1 ug/ul |
| | | Cell BIO- | (0N3R) | | |
| | | RAD | | | |
| Tau441 | | >90% by | Adult | 45.9 | 1 ug/ul |
| | | SDS-PAGE | (2N4R) | | |
| | | (rPeptide) | | | |

RNA and tau were incubated in 1× protection assay buffer (“PAB”) in 10 ul final volume. Control reactions included RNAs without tau, tau in 1×PAB, tau in Laemmli Buffer (with SDS).

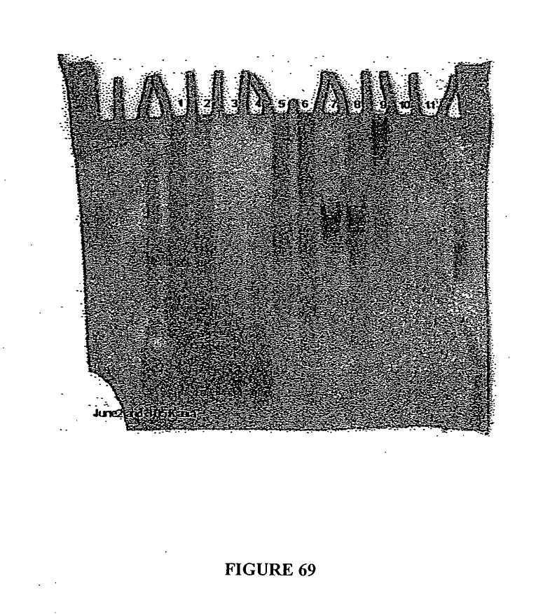

FIG. 69 shows a gel showing the results of RQ11+12, MNV, AP1 and tRNA with and without Tau23. The columns of the gel were set up as follows:

| | Tau23 | RQ11+12 | MNV | AP1 | tRNA | 10x PAB | |
|---|---|---|---|---|---|---|---|
| No. | (1 ug/ul) | (125 ng/ul) | (125 ng/ul) | (114 ng/ul) | (125 ng/ul) | pH 7.4 | H₂O |
| 1 | | 1 ul | | | | 1 ul | 8 ul |
| 2 | 1 ul | 1 ul | | | | 1 ul | 7 ul |
| 3 | | | 1 ul | | | 1 ul | 8 ul |
| 4 | 1 ul | | 1 ul | | | 1 ul | 7 ul |
| 5 | | | | 3 ul | | 1 ul | 6 ul |
| 6 | 1 ul | | | 3 ul | | 1 ul | 5 ul |
| 7 | | | | | 1 ul | 1 ul | 8 ul |
| 8 | 1 ul | | | | 1 ul | 1 ul | 7 ul |
| 9 | 1 ul | 1 ul | | | | 1 ul | 7 ul |
| | rPeptide | | | | | | |
| | Tau | | | | | | |
| | 1 ug/ul | | | | | | |
| 10 | 1 ul | | | | | 1 ul | 8 ul |
| 11 | 1 ul | | | | | | 9 ul 2x |
| | | | | | | | Laemmli |
| | | | | | | | Buffer |

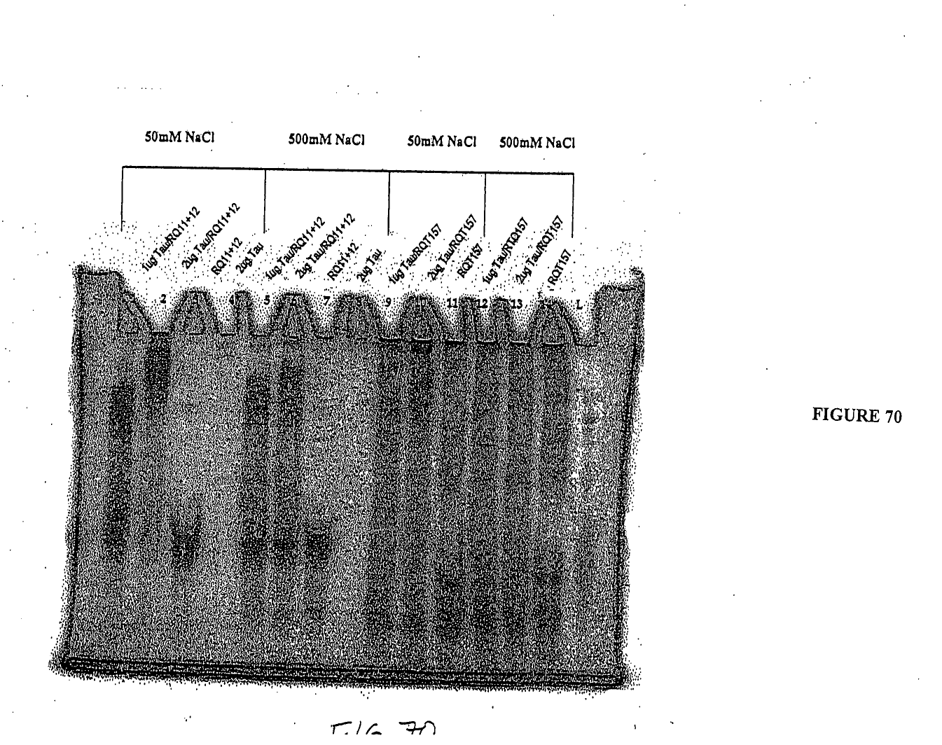

FIG. 70 shows a gel showing the results of RQ11+12, MNV, AP1 and tRNA with and without Tau441. The columns of the gel were set up as follows:

| | rPeptide | | | | | | |
|---|---|---|---|---|---|---|---|
| | Tau | tRNA | AP1 | MNV | RQ11+12 | 10x PAB | |
| No. | (1 ug/ul) | (37.5 ng/ul) | (114 ng/ul) | (125 ng/ul) | (125 ng/ul) | pH 7.4 | H₂O |
| 1 | | 3.3 ul | | | | 1 ul | 5.7 ul |
| 2 | 1 ul | 3.3 ul | | | | 1 ul | 4.7 ul |
| 3 | | | 3 ul | | | 1 ul | 6 ul |
| 4 | 1 ul | | 3 ul | | | 1 ul | 5 ul |
| 5 | | | | 1 ul | | 1 ul | 8 ul |
| 6 | 1 ul | | | 1 ul | | 1 ul | 7 ul |
| 7 | | | | | 1 ul | 1 ul | 8 ul |
| 8 | 1 ul | | | | 1 ul | 1 ul | 7 ul |
| 9 | 1 ul tau23 | 1 ul | | | | 1 ul | 7 ul |
| | 1 ug/ul | | | | | | |
| 10 | 1 ul | | | | | 1 ul | 8 ul |
| 11 | 1 ul | | | | | | 9 ul |

The reactions were run at room temperature for 30 mins. 4 ul of 6× Loading Buffer Fermentas was added. The samples were loaded on a 15 well, 6% PA/TBE/7M Urea gel Novex from Invitrogen. The gel was run at 80V. for 2.5 hours on ice, using 1×TBE as running buffer.

The gels were then stained with SybfGold for 15 mins and scanned on FluorImager595 with filter F570FD30.

In addition to showing binding of tau to different RNAs, a comparison of the gels in FIGS. 69 and 70 show that the two different tau proteins do not have similar binding affinities for the RNAs.

### Example 22

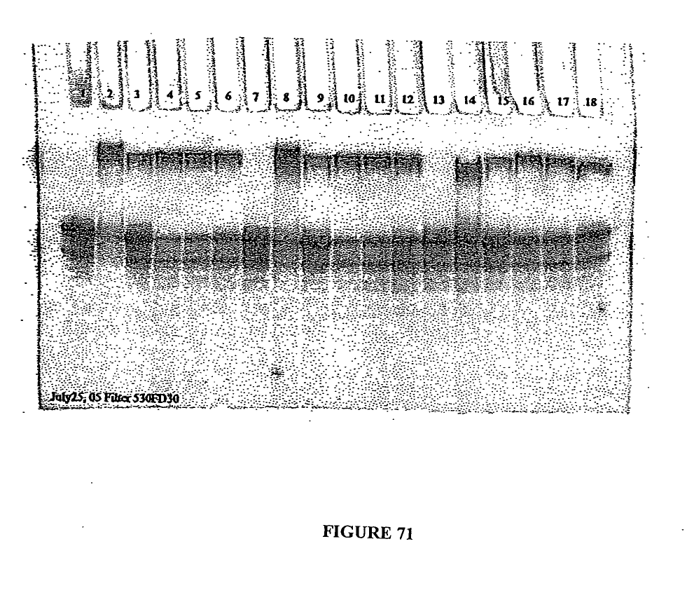

The effect of salt concentration on the binding of tau to RNA was examined. 125 ng. of RQ11+12 or RQT157 was incubated with 1 ug or 2 ug of tau (tau23) protein in 50 mM or 500 mM final concentrations of NaCl, with 10× PAB minus LiCl. Reaction was in a total volume of 10 ul and incubated for 30 minutes at room temperature followed by 4 ul of 6× Mass loading dye from Fermentas. Samples were loaded onto a 6% TBE/7M urea gel from Invitrogen and eletrophoresed on ice at 80V for 30 minutes followed by 1.5 hrs. at 180V. The gel was then stained with SYBR gold, photographed and scanned. See FIG. 71.

| | | PAB | 500 mM | | Tau | RQ11+12 | RQT157 |
|---|---|---|---|---|---|---|---|
| No. | H₂O | (—LiCl) | NaCl | 5M NaCl | (1 ug/ul) | (125 ng/ul) | (125 ng/ul) |
| 1 | 6 ul | 1 ul | 1 ul | | 1 ul | 1 ul | |
| 2 | 5 ul | 1 ul | 1 ul | | 2 ul | 1 ul | |
| 3 | 7 ul | 1 ul | 1 ul | | | 1 ul | |
| 4 | 6 ul | 1 ul | 1 ul | | 2 ul | | |
| 5 | 6 ul | 1 ul | | 1 ul | 1 ul | 1 ul | |
| 6 | 5 ul | 1 ul | | 1 ul | 2 ul | 1 ul | |
| 7 | 7 ul | 1 ul | | 1 ul | | 1 ul | |
| 8 | 6 ul | 1 ul | | 1 ul | 2 ul | | |
| 9 | 5 ul | 1 ul | 1 ul | | 1 ul | | 1 ul |
| 10 | 6 ul | 1 ul | 1 ul | | 2 ul | | 1 ul |
| 11 | 7 ul | 1 ul | 1 ul | | | | 1 ul |
| 12 | 6 ul | 1 ul | | 1 ul | 1 ul | | 1 ul |
| 13 | 5 ul | 1 ul | | 1 ul | 2 ul | | 1 ul |
| 14 | 7 ul | 1 ul | | 1 ul | | | 1 ul |

| | |
|---|---|
| 15 | Protein Ladder |
| | Protein Ladder |

RQ11+12 and RQT157 show specificity for tau under stringent conditions. The binding of tau to RQT157 in this example appears to be dependent on chart interaction. On the other hand, the binding of tau to RQ11+12, in this example, is not solely dependent on charge interaction. High salt conditions produced RQ11+12/Tau binding specificity at 1:15 molar ratio, which is absent in RQT157/tau samples, hence allowing differentiation between molecules.

### Example 23

Using competitions assays and increasing salt stringencies, RQ11+12 was shown to demonstrate greater specificity of interaction for tau than RQT157. The competition reactions were run using fluorescently labeled RQ11+12 and unlabeled RQT157 and tau 50 mM, 150 mM and 500 MM NaCl.

| | | 10x | | RQ11+12* | RQ11+12 | RQT157 | Tau |
|---|---|---|---|---|---|---|---|
| No. | H₂O | PAB | NaCl | (120 ng/ul) | (1 ug/ul) | (1.2 ug/ul) | (1 ug/ul) |
| 1 | 7 ul | 1 ul | 1 ul (500 mM) | 1 ul | — | — | — |
| 2 | 5 ul | 1 ul | 1 ul (500 mM) | 1 ul | — | — | 2 ul |
| 3 | — | 1 ul | 1 ul (500 mM) | 1 ul | 5 ul | — | 2 ul |
| 4 | 4.5 ul | 1 ul | 1 ul (500 mM) | 1 ul | — | 0.5 ul | 2 ul |
| 5 | 4 ul | 1 ul | 1 ul (500 mM) | 1 ul | — | 1 ul | 2 ul |
| 6 | — | 1 ul | 1 ul (500 mM) | 1 ul | — | 5 ul | 2 ul |
| 7 | 7 ul | 1 ul | 1 ul (1500 mM) | 1 ul | — | — | — |
| 8 | 5 ul | 1 ul | 1 ul (1500 mM) | 1 ul | — | — | 2 ul |
| 9 | — | 1 ul | 1 ul (1500 mM) | 1 ul | 5 ul | — | 2 ul |
| 10 | 4.5 ul | 1 ul | 1 ul (1500 mM) | 1 ul | — | 0.5 ul | 2 ul |
| 11 | 4 ul | 1 ul | 1 ul (1500 mM) | 1 ul | — | 1 ul | 2 ul |
| 12 | — | 1 ul | 1 ul (1500 mM) | 1 ul | — | 5 ul | 2 ul |
| 13 | 7 ul | 1 ul | 1 ul (5000 mM) | 1 ul | — | — | — |
| 14 | 5 ul | 1 ul | 1 ul (5000 mM) | 1 ul | — | — | 2 ul |
| 15 | — | 1 ul | 1 ul (5000 mM) | 1 ul | 5 ul | — | 2 ul |
| 16 | 4.5 ul | 1 ul | 1 ul (5000 mM) | 1 ul | — | 0.5 ul | 2 ul |
| 17 | 4 ul | 1 ul | 1 ul (5000 mM) | 1 ul | — | 1 ul | 2 ul |
| 18 | — | 1 ul | 1 ul (5000 mM) | 1 ul | — | 5 ul | 2 ul |

### Example 24

Electron microscopy was used to examine what types of structures are being produced by in vitro-Tripartite. FIGS. 72A-72B are electron micrographs of 4 uM of Tau412 in 1× fibril buffer after 3 hours of incubation. Tau protein aggregates and fibrils are formed in a mildly denaturing environment of DTT (Dithiothreitol) and in the presence of an shsRNA (in this case RQ11+12 and RQT157). Different species of aggregated/fibrilized tau are created in the presence of different shsRNAs. The invitro system resmbles the pathology of a protein misfolding disease.

1-103. (canceled)

104. A TRIPARTITE system comprising:
(a) a cellular binding factor; and
(b) an isolated RNA; and
(c) a cellular isoform of a protein associated with a protein misfolding disease.

105. The TRIPARTITE system of claim 104, wherein said cellular binding factor is fibronectin.

106. The TRIPARTITE system of claim 104, wherein said RNA is RQ11+12.

107. The TRIPARTITE system of claim 104, wherein said protein is a Tau protein.

108. A method of mimicking a protein misfolding disease cascade comprising:
contacting a cellular binding factor and an isolated RNA to a cellular isoform of a protein associated with a protein misfolding disease, and providing sufficient time to allow the cellular binding factor and the isolated RNA each to bind to the cellular isoform of the protein, thereby causing cellular isoform of the protein to misfold into a disease isoform of the protein.

109. The method of claim 108, wherein said cellular binding factor is fibronectin.

110. The method of claim 108, wherein said RNA is RQ11+12.

111. The method of claim 108, wherein said protein is a Tau protein.

112. The method of claim 108, wherein said protein misfolding disease is Alzheimer's disease.

113. A method of evaluating a therapy or treatment of protein misfolding diseases, comprising:
(a) contacting a cellular binding factor and an isolated RNA to a cellular isoform of a protein associated with a protein misfolding disease to create a mixture;
(b) applying the therapy or treatment to the mixture;
(c) detecting the presence or absence of a disease isoform of the protein, the absence of the disease isoform of the protein indicating that the therapy or treatment is effective.

114. The method of claim 113, wherein the cellular binding factor is fibronectin.

115. The method of claim 113, wherein the RNA is RQ11+12.

116. The method of claim 113, wherein the protein is a Tau protein.

117. The method of claim 113, wherein said protein misfolding disease is Alzheimer's disease.
